(19) United States
(12) Patent Application Publication
(10) Pub. No.: US 2017/0326234 A1
(43) Pub. Date: Nov. 16, 2017

(54) COMBINATION THERAPIES

(71) Applicant: Celgene Corporation, Summit, NJ (US)

(72) Inventors: Markus RENSCHLER, Redwood City, CA (US); Mark ALLES, Dallas, PA (US)

(73) Assignee: Celgene Corporation, Summit, NJ (US)

(21) Appl. No.: 15/531,954

(22) PCT Filed: Dec. 1, 2015
(86) PCT No.: PCT/US15/63194

§ 371 Date: May 31, 2017

Related U.S. Application Data
(60) Provisional application No. 62/086,533, filed on Dec. 2, 2014.

(51) Int. Cl. A61K 39/395; A61K 47/69; A61K 31/337; A61K 31/7068; C07K 16/28; A61K 47/64; A61K 33/24; A61K 38/17; A61K 31/555

(52) U.S. Cl. CPC A61K 39/3955; A61K 38/1774; A61K 47/6931; A61K 31/337; A61K 31/555; C07K 16/2827; C07K 16/2818; A61K 47/643; A61K 33/24; A61K 31/7068; C07K 2317/21; C07K 2317/24; C07K 2317/76; C07K 2319/30

(57) ABSTRACT

The present invention provides combination therapy methods of treating a proliferative disease (such as cancer) comprising a first therapy comprising administering to an individual an effective amount of a taxane in a nanoparticle composition, and a second therapy which may include the administration of an effective amount of at least one other agent that antagonizes a PD-1 pathway in a cell.

### CROSS-REFERENCE TO RELATED APPLICATIONS

This application claims the benefit of U.S. Provisional Application No. 62/086,533, filed on Dec. 2, 2014, which is hereby incorporated by reference in its entirety.

### I. FIELD

Provided herein are methods and compositions for the treatment of proliferative diseases comprising the administration of a combination of a taxane and at least one agent that antagonizes a PD-1 pathway in a cell.

### II. BACKGROUND

Cancer is a leading cause of death world wide. Despite significant advances in the field of chemotherapy, many of the most prevalent forms of cancer still resist chemotherapeutic intervention. The incidence of cancer continues to climb as the general population ages and as new forms of cancer develop.

Taxanes (such as paclitaxel and docetaxel) have been shown to have significant antineoplastic and anticancer effects in a wide variety of cancers. For example, paclitaxel acts by interfering with the normal function of microtubule breakdown. Paclitaxel binds to the beta subunit of tubulin, the building blocks of microtubules, causing hyper-stabilization of the microtubule structures. The resulting paclitaxel/microtubule structure is unable to disassemble, thereby arresting mitosis and inhibiting angiogenesis. The poor aqueous solubility for the taxanes, however, presents significant challenges for developing effective taxane-based cancer therapeutics. Furthermore, the interaction of different taxane formulations with other therapeutic agents in the combination therapy context remains to be studied.

Albumin-based nanoparticle compositions have been developed as a drug delivery system for delivering substantially water insoluble drugs such as taxanes. See, for example, U.S. Pat. Nos. 5,916,596; 6,506,405; 6,749,868, and 6,537,579, and 7,820,788 and also in U.S. Pat. Pub. Nos. 2007/0082838. The albumin-based nanoparticle technology utilizes the natural properties of the protein albumin to transport and deliver substantially water insoluble drugs to the site of disease. These nanoparticles are readily incorporated into the body's own transport processes and are able to exploit the tumors' attraction to albumin, enabling the delivery of higher concentrations of the active drug encapsulated in the nanoparticles to the target site. In addition, the albumin-based nanoparticle technology offers the ability to improve a drug's solubility by avoiding the need for toxic chemicals, such as solvents, in the administration process, thus potentially improving safety through the elimination of solvent-related side effects.

Other references include PCT Application Nos. WO08/057562, WO2009126938A1, WO2009126401A1, WO2009126175A1.

A continuing need exists for effective therapies to treat subjects with cancer.

The disclosures of all publications, patents, patent applications and published patent applications referred to herein are hereby incorporated herein by reference in their entirety.

### III. SUMMARY

In one aspect, provided herein are combination therapy methods of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell (also referred herein as a “PD-1 pathway antagonist,” “antagonist of the PD-1 pathway,” “PD-1 pathway inhibitor,” or “the other agent”). In some embodiments, provided herein is a method of treating a proliferative disease (such as cancer) in an individual comprising administering to the individual a) an effective amount of a composition comprising nanoparticles comprising paclitaxel coated with an albumin (such as nab-paclitaxel, e.g., ABRAXANE®), and b) an effective amount of at least one other agent that antagonizes a PD-1 pathway in a cell.

In some embodiments, the proliferative disease is resistant or refractory to the treatment of taxane when administered alone or in conjunction with an agent other than the PD-1 pathway antagonist. In some embodiments, the proliferative disease is resistant or refractory to the treatment when the PD-1 pathway antagonist is administered alone or in conjunction with an agent other than the nanoparticle composition (such as a non-nanoparticle composition of a taxane including paclitaxel).

In some embodiments, the composition comprising nanoparticles (also referred to as “nanoparticle composition”) and the other agent are administered simultaneously, either in the same composition or in separate compositions. In some embodiments, the nanoparticle composition and the other agent are administered sequentially, i.e., the nanoparticle composition is administered either prior to or after the administration of the other agent.

In some embodiments, the administration of the nanoparticle composition and the other agent is concurrent, i.e., the administration period of the nanoparticle composition and that of the other agent overlap with each other. In some embodiments, the nanoparticle composition is administered for at least one cycle (for example, at least any of 2, 3, or 4 cycles) prior to the administration of the other agent. In some embodiments, the other agent is administered for at least any of one, two, three, or four weeks after the termination of the nanoparticle composition. In some embodiments, the nanoparticle composition and the PD-1 pathway antagonist are administered over the same treatment cycles.

In some embodiments, the administration of the nanoparticle composition and the other agent are non-concurrent. For example, in some embodiments, the administration of the nanoparticle composition is terminated before the other agent is administered. In some embodiments, the administration of the other agent is terminated before the nanoparticle composition is administered.

In some embodiments, the other agent is an antibody. In some embodiments, the antibody is a monoclonal antibody. In some embodiments, the antibody is a human antibody. In some embodiments, the antibody is a humanized antibody. In some embodiments, the antibody is a fully human antibody. In some embodiments, the antibody is a chimeric antibody. In some embodiments, the antibody targets a component of the PD-1 pathway. For example, in some embodiments, the antibody is an anti-PD-1 antibody. In some embodiments, the antibody is an anti-PD-L1 antibody. In some embodiments, the antibody is an anti-PD-L2 antibody.

In some embodiments, the other agent comprises at least a portion of an immunoglobulin (Ig). In some embodiments, the immunoglobulin (Ig) is immunoglublin G (IgG). In some embodiments, the IgG is IgG1. In some embodiments, the IgG is IgG2. In some embodiments, the IgG is IgG3. In some embodiments, the IgG is IgG4. In some embodiments, the IgG is a human IgG.

In some embodiments, the other agent antagonizes PD-1, a ligand of PD-1 (such as PD-L1 or PD-L2), or a combination thereof. In some embodiments, the other agent is selected from the group consisting of AMP-224, nivolumab, AMP-514, pembrolizumab, pidilizumab, REGN2810, PDR001, BGB-A317, BMS-936559, MEDI4736, MSB0010718C, and MPDL3280A In some embodiments, the other agent is nivolumab. In some embodiments, the other agent is pembrolizumab. In some embodiments, the other agent is pidilizumab. In some embodiments, the other agent is REGN2810. In some embodiments, the other agent is PDR001. In some embodiments, the other agent is BGB-A317. In some embodiments, the other agent is AMP-514. In some embodiments, the other agent is AMP-224.

In some embodiments, the other agent is an antagonist of PD-1 (also referred herein as a “PD-1 antagonist” or “PD-1 inhibitor”). In some embodiments, the antagonist of PD-1 disrupts or interferes with the interaction between PD-1 and a ligand of PD-1. In some embodiments, the antagonist of PD-1 disrupts or interferes with the interaction between PD-1 and PD-L1. In some embodiments, the antagonist of PD-1 disrupts or interferes with the interaction between PD-1 and PD-L2. In some embodiments, the PD-1 antagonist comprises a fusion protein. In some embodiments, the fusion protein is AMP-224. In some embodiments, the PD-1 antagonist comprises an antibody. In some embodiments, the PD-1 antagonist comprises an anti-PD-1 antibody. In some embodiments, a PD-1 antagonist is selected from the group consisting of nivolumab (i.e., BMS-936558), AMP-514, pembrolizumab (i.e., MK-3475), REGN2810, PDR001, BGB-A317, and pidilizumab (i.e., CT-011). In some embodiments, the anti-PD-1 antagonist is nivolumab. In some embodiments, the anti-PD-1 antagonist is pembrolizumab. In some embodiments, the anti-PD-1 antagonist is REGN2810. In some embodiments, the anti-PD-1 antagonist is PDR001. In some embodiments, the anti-PD-1 antagonist is BGB-A317.

In some embodiments, the other agent is an antagonist of PD-L1 (also referred herein as a “PD-L1 antagonist” or “PD-L1 inhibitor”). In some embodiments, the antagonist of PD-L1 disrupts or interferes with the interaction between PD-1 and PD-L1. In some embodiments, the PD-L1 antagonist comprises a fusion protein. In some embodiments, the fusion protein is AMP-224. In some embodiments, the PD-L1 antagonist comprises an antibody. In some embodiments, the PD-L1 antagonist comprises an anti-PD-L1 antibody. In some embodiments, a PD-L1 antagonist is selected from the group consisting of BMS-936559, MEDI4736, MSB0010718C (i.e., avelumab) and MPDL3280A (i.e., RG7446). In some embodiments, a PD-L1 antagonist is MEDI4736. In some embodiments, a PD-L1 antagonist is MPDL3280A. In some embodiments, a PD-L1 antagonist is MSB0010718C.

In some embodiments, the other agent is an antagonist of PD-L2. In some embodiments, the antagonist of PD-L2 disrupts or interferes with the interaction between PD-1 and PD-L2. In some embodiments, the PD-L2 antagonist comprises a fusion protein. In some embodiments, the fusion protein is AMP-224. In some embodiments, the PD-L2 antagonist comprises an antibody. In some embodiments, the PD-L2 antagonist comprises an anti-PD-L2 antibody.

In some embodiments, the other agent comprises a fusion protein. In some embodiments, the fusion protein comprises at least a portion of an antibody. In some embodiments, the fusion protein comprises at least a portion of a non-antibody protein. In some embodiments, the fusion protein comprises at least a portion of an antibody and at least a portion of a non-antibody protein. In some embodiments, the antibody targets a component of the PD-1 pathway. For example, in some embodiments, the antibody targets PD-1. In some embodiments, the antibody targets a ligand of PD-1 (e.g., PD-L1, PD-L2). In some embodiments, the non-antibody protein comprises a component of the PD-1 pathway. For example, in some embodiments, the non-antibody protein comprises at least a portion of a ligand of PD-1. In some embodiments, the non-antibody portion comprises at least a portion of PD-L1. In some embodiments, the non-antibody portion comprises at least a portion of PD-L2. In some embodiments, the non-antibody portion comprises at least a portion of PD-1. In some embodiments, the other agent is AMP-224.

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of nivolumab (BMS-936558).

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of AMP-514.

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of pembrolizumab (MK-3475).

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of pidilizumab (CT-011).

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of REGN2810.

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of PDR001.

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of BGB-A317.

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of BMS-936559.

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of MEDI4736.

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of MPDL3280A (RG7446).

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of MSB0010718C (avelumab).

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of AMP-224.

In some embodiments, there is provided a method of treating breast cancer in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for HER2, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for ER, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for PR, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for HER2 and ER, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for HER2 and PR, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for ER and PR, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating breast cancer in an individual, wherein the individual is negative for ER, PR, and HER2, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating non-small cell lung cancer in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments, there is provided a method of treating pancreatic cancer in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell.

In some embodiments of the methods disclosed herein, the method further comprises conducting definitive surgery within about 1, 2, 3, 4, 5, 6, 7, 8, 9, or 10 following the preoperative therapy.

The methods presented herein generally comprise administration of a composition comprising nanoparticles comprising a taxane (such as paclitaxel) and a carrier protein. In some embodiments, the composition comprises nanoparticles comprising paclitaxel and an albumin. In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of all the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of all the nanoparticles in the composition fall within the range of about 20 to about 400, including for example about 20 to about 200 nm, about 30 to about 180 nm, and any one of about 40 to about 150, about 50 to about 120, and about 60 to about 100 nm. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the nanoparticle has an average diameter of less than or equal to about 200 nm and the weight ratio of the carrier protein (such as albumin) to taxane (such as paclitaxel) in the composition is about 9:1.

In some embodiments, the carrier protein has sulfhydral groups that can form disulfide bonds. In some embodiments, at least about 5% (including for example at least about any one of 10%, 15%, 20%, 25%, 30%, 40%, 50%, 60%, 70%, 80%, or 90%) of the carrier protein in the nanoparticle portion of the composition are crosslinked (for example crosslinked through one or more disulfide bonds).

In some embodiments, the nanoparticles comprise the taxane (such as paclitaxel) coated with a carrier protein, such as albumin (e.g., human serum albumin). In some embodiments, the composition comprises taxane in both nanoparticle and non-nanoparticle form, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles comprise a core of taxane that is substantially free of polymeric materials (such as polymeric matrix).

In some embodiments, the nanoparticle composition is substantially free (such as free) of surfactants (such as CREMOPHOR®, Tween 80, or other organic solvents used for the administration of taxanes). In some embodiments, the nanoparticle composition contains less than about any one of 20%, 15%, 10%, 7.5%, 5%, 2.5%, or 1% organic solvent. In some embodiments, the weight ratio of carrier protein (such as albumin) to taxane (such as paclitaxel) in the nanoparticle composition is about 18:1 or less, such as about 15:1 or less, for example about 9:1 or less. In some embodiments, the weight ratio of carrier protein (such as albumin) to taxane (such as paclitaxel) in the composition falls within the range of any one of about 1:1 to about 18:1, about 2:1 to about 15:1, about 3:1 to about 13:1, about 4:1 to about 12:1, about 5:1 to about 10:1, about 9:1. In some embodiments, the weight ratio of carrier protein (such as albumin) to taxane (such as paclitaxel) in the nanoparticle portion of the composition is about any one of 2:1, 3:1, 4:1, 5:1, 9:1, 10:1, 15:1, or less.

In some embodiments, the nanoparticle composition comprises one or more of the above characteristics. In some embodiments, the nanoparticle composition is a nanoparticle composition described in Sections V-C and V-D, infra. In some embodiments, the nanoparticle composition comprises any of the nanoparticle components described in Section V-D1, infra.

In some embodiments, the nanoparticle composition is ABRAXANE®. Nanoparticle compositions comprising other taxanes (such as tesetaxel, docetaxel, and ortataxel) may also comprise one or more of the above characteristics.

In one aspect, presented herein is a method of treating a proliferative disease in an individual, comprising administering to the individual: a) an effective amount of a composition comprising nab-paclitaxel, and b) an effective amount of an anti-PD-1 antibody. In some embodiments, the composition in a) comprises ABRAXANE®. In some embodiments, the anti-PD-1 antibody is nivolumab. In some embodiments, the proliferative disease is lung cancer. In some embodiments, the proliferative disease is pancreatic cancer. In some embodiments, the proliferative disease is breast cancer.

In some embodiments, the further comprising administering to the individual an effective amount of a platinum-based agent. In some embodiments, the platinum-based agent is carboplatin. In some embodiments, the platinum-based agent is cisplatin. In some embodiments, the composition a) and the anti-PD-1 antibody are administered concurrently. In some embodiments, the composition in a) and the anti-PD-1 antibody are administered sequentially. In some embodiments, the composition in a) and the anti-PD-1 antibody are administered simultaneously. In some embodiments, the composition in a), the anti-PD-1 antibody and the platinum-based agent are administered concurrently.

In some embodiments, the composition in a), the anti-PD-1 antibody and the platinum-based agent are administered concurrently are administered sequentially. In some embodiments, the composition in a), the anti-PD-1 antibody and the platinum-based agent are administered concurrently are administered simultaneously.

In some embodiments, there is provided a method of treating breast cancer (such as HER2 negative breast cancer) in an individual, comprising administering to said individual a) an effective amount of nab-paclitaxel, e.g., ABRAXANE®, and b) an effective amount of a PD-1 pathway inhibitor. In some embodiments, there is provided a method of treating breast cancer (such as HER2 negative breast cancer) in an individual, comprising administering to said individual a) an effective amount of ABRAXANE®, and b) an effective amount of an antagonist of PD-1. In some embodiments, there is provided a method of treating breast cancer (such as HER2 negative breast cancer) in an individual, comprising administering to said individual a) an effective amount of ABRAXANE®, and b) an effective amount of an anti-PD-1 antibody. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the amount of ABRAXANE® administered to the individual is an effective amount described in Section V-C, infra. In some embodiments, the amount of ABRAXANE® administered to the individual is about 45 mg/m² to about 350 mg/m². In some embodiments, the amount of ABRAXANE® administered to the individual is about 50 mg/m² to about 300 mg/m². In some embodiments, the amount of ABRAXANE® administered to the individual is about 50 mg/m² to about 350 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, the PD-1 pathway inhibitor is an anti-PD-1 antibody. In some embodiments, the antagonist of PD-1 is an anti-PD-1 antibody. In some embodiments, the anti-PD-1 antibody is selected from the group consisting of nivolumab (BMS-936558), AMP-514, pembrolizumab (MK-3475), REGN2810, PDR001, BGB-A317, and pidilizumab (CT-011). In some embodiments, the anti-PD-1 antibody is nivolumab. In some embodiments, the nivolumab and nab-paclitaxel, e.g., ABRAXANE®, is administered to the individual in a dosing schedule as described in Section V-C, infra. In some embodiments, the nivolumab and nab-paclitaxel, e.g., ABRAXANE®, is administered to the individual at a dose or dose range described in Section V-C, infra. In some embodiments, the nivolumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is pembrolizumab. In some embodiments, the pembrolizumab and nab-paclitaxel, e.g., ABRAXANE®, is administered to the individual in a dosing schedule as described in Section V-C, infra. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, the pidilizumab and nab-paclitaxel, e.g., ABRAXANE®, is administered to the individual in a dosing schedule as described in Section V-C, infra. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the REGN2810 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the nab-paclitaxel, e.g., ABRAXANE®, is administered as described hereinabove. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the BGB-A317 and nab-paclitaxel, e.g., ABRAXANE®, is administered to the individual in a mode of administration described in Section V-C, infra. In some embodiments, the nab-paclitaxel, e.g., ABRAXANE®, is adminstered as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the anti-PD-1 antibody is AMP-514. In some embodiments, the second agent is AMP-514. In some embodiments, AMP-514 and nab-paclitaxel, e.g., ABRAXANE®, is administered to the individual in a dosing schedule as described in Section V-C, infra. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a mode of administration described in Section V-C, infra. In some embodiments, the nab-paclitaxel, e.g., ABRAXANE®, In some embodiments, between about 0.1 mg/kg to about 30 mg/kg of AMP-514 is administered. In some embodiments, between about 0.5 mg/kg to about 30 mg/kg of AMP-514 is administered. In some embodiments, between about 0.5 mg/kg to about 20 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the nab-paclitaxel, e.g., ABRAXANE®, is administered intravenously. In some embodiments, the ABRAXANE® is administered intravenously. In some embodiments, the PD-1 pathway inhibitor is administered intravenously. In some embodiments, the antagonist of PD-1 is administered intravenously. In some embodiments, the anti-PD-1 antibody is administered intravenously. In some embodiments, the nab-paclitaxel, e.g., ABRAXANE®, and the anti-PD-1 antibody are administered intravenously. In some embodiments, the ABRAXANE® and the anti-PD-1 antibody are administered intravenously. In some embodiments, the breast cancer being treated is a HER2 negative breast cancer. In particular embodiments, the breast cancer being treated is a triple negative breast cancer (clinically negative for expression of estrogen receptor (ER), progesterone receptor (PR) and Her2/neu).

In some embodiments, there is provided a method of treating breast cancer (such as HER2 negative breast cancer) in an individual, comprising: a) intravenously administering about 50 to about 350 mg/m² (such as about 75 mg/m², 100 mg/m², 125 mg/m², 150 mg/m², 200 mg/m², 225 mg/m², 250 mg/m², 260 mg/m²) of ABRAXANE® to the individual weekly, and b) intravenously administering about 0.01 to about 10 mg/kg (such as about 3 mg/kg) of an anti-PD-1 antibody (such as nivolumab (BMS-936558), AMP-514, pembrolizumab (MK-3475), REGN2810, PDR001, BGB-A317, or pidilizumab (CT-011)) to the individual one out of every fourteen days. In some embodiments, the nanoparticle composition is administered on days one, eight and fifteen of every twenty eight days, and an anti-PD-1 antibody (such as nivolumab (BMS-936558), AMP-514, pembrolizumab (MK-3475), REGN2810, PDR001, BGB-A317, or pidilizumab (CT-011)) is administered on days 1 and 15 of every twenty eight days. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the amount of ABRAXANE® administered to the individual is an effective amount described in Section V-C, infra. In some embodiments, the amount of ABRAXANE® administered to the individual is about 50 mg/m² to about 300 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, the anti-PD-1 antibody is selected from the group consisting of nivolumab (BMS-936558), AMP-514, pembrolizumab (MK-3475), REGN2810, PDR001, BGB-A317, and pidilizumab (CT-011). In some embodiments, the anti-PD-1 antibody is nivolumab. In some embodiments, the nivolumab and nab-paclitaxel, e.g., ABRAXANE®, is administered to the individual at a dose or dose range described in Section V-C, infra. In some embodiments, the nivolumab and nab-paclitaxel, e.g., ABRAXANE®, is administered to the individual in a dosing schedule as described in Section V-C, infra. In some embodiments, the nab-paclitaxel, e.g., ABRAXANE®, is administered as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a dosing schedule as described in Section V-C, infra. In some embodiments, the nab-paclitaxel, e.g., ABRAXANE®, is administered as described hereinabove. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a dosing schedule as described in Section V-C, infra. In some embodiments, the nab-paclitaxel, e.g., ABRAXANE®, is administered as described hereinabove. In some embodiments, between about 0.01 mg/kg to about 10 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a dosing schedule as described in Section V-C, infra. In some embodiments, the nab-paclitaxel, e.g., ABRAXANE®, is administered as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the nab-paclitaxel, e.g., ABRAXANE®, is administered as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the nab-paclitaxel, e.g., ABRAXANE®, is administered as described hereinabove. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the breast cancer being treated is a HER2 negative breast cancer. In particular embodiments, the breast cancer being treated is a triple negative breast cancer (negative for estrogen receptor (ER), progesterone receptor (PR) and Her2/neu).

In some embodiments, there is provided a method of treating lung cancer (such as NSCLC) in an individual, comprising administering to said individual a) an effective amount of ABRAXANE®, and b) an effective amount of a PD-1 pathway antagonist. In some embodiments, there is provided a method of treating lung cancer (such as NSCLC) in an individual, comprising administering to said individual a) an effective amount of ABRAXANE®, and b) an effective amount of an antagonist of PD-1. In some embodiments, there is provided a method of treating lung cancer (such as NSCLC) in an individual, comprising administering to said individual a) an effective amount of ABRAXANE®, and b) an effective amount of an anti-PD-1 antibody (such as nivolumab (BMS-936558), AMP-514, pembrolizumab (MK-3475), REGN2810, PDR001, BGB-A317, or pidilizumab (CT-011)). In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the amount of ABRAXANE® administered to the individual is an effective amount described in Section V-C, infra. In some embodiments, the amount of ABRAXANE® administered to the individual is about 45 mg/m² to about 350 mg/m². In some embodiments, the amount of ABRAXANE® administered to the individual is about 50 mg/m² to about 300 mg/m². In some embodiments, the amount of ABRAXANE® administered to the individual is about 50 mg/m² to about 350 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, the nab-paclitaxel, e.g., ABRAXANE®, is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, the anti-PD-1 antibody is nivolumab. In some embodiments, the nivolumab and nab-paclitaxel, e.g., ABRAXANE®, is administered to the individual at a dose or dose range described in Section V-C, infra. In some embodiments, the nivolumab and nab-paclitaxel, e.g., ABRAXANE®, is administered to the individual in a dosing schedule as described in Section V-C, infra. In some embodiments, the nivolumab nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a dosing schedule as described in Section V-C, infra. In some embodiments, AMP-514 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, the anti-PD-1 antibody is pembrolizumab. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a dosing schedule as described in Section V-C, infra. In some embodiments, pembrolizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a dosing schedule as described in Section V-C, infra. In some embodiments, pidilizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the REGN2810 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the PDR001 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the BGB-A317 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the nab-paclitaxel, e.g., ABRAXANE®, is administered intravenously. In some embodiments, the ABRAXANE® is administered intravenously. In some embodiments, the PD-1 pathway inhibitor is administered intravenously. In some embodiments, the antagonist of PD-1 is administered intravenously. In some embodiments, the anti-PD-1 antibody is administered intravenously. In some embodiments, the nab-paclitaxel, e.g., ABRAXANE®, and the anti-PD-1 antibody are administered intravenously. In some embodiments, the ABRAXANE® and the anti-PD-1 antibody are administered intravenously. In some embodiments, the method further comprises administering to the individual an effective amount of a platinum-based agent. In some embodiments, the platinum-based agent is carboplatin. In some embodiments, the platinum-based agent is administered intravenously. In some embodiments, the carboplatin is administered intravenously. In some embodiments, the cisplatin is administered intravenously. In some embodiments, the ABRAXANE®, the anti-PD-1 antibody and the platinum-based agent are administered intravenously. In some embodiments, the platinum-based agent is cisplatin. In some embodiments, the lung cancer is non-small cell lung cancer (NSCLC).

In some embodiments, there is provided a method of treating lung cancer (such as NSCLC) in an individual, comprising: a) intravenously administering about 50 to about 350 mg/m² (such as about 75 mg/m², 100 mg/m², 125 mg/m², 150 mg/m², 200 mg/m², 225 mg/m², 250 mg/m²) of ABRAXANE® to the individual weekly, and b) intravenously administering about 0.01 to about 10 mg/kg (such as about 3 mg/kg) of an anti-PD-1 antibody (such as nivolumab (BMS-936558), AMP-514, pembrolizumab (MK-3475), REGN2810, PDR001, BGB-A317, or pidilizumab (CT-011)) to the individual one out of every twenty one days. In some embodiments, the method further comprises intravenously administering to the individual an effective amount of carboplatin at the dose of AUC6 one out of every twenty one days. In some embodiments, the nanoparticle composition is administered on days 1, 8 and 15 of every 21 days, and an anti-PD-1 antibody (such as nivolumab (BMS-936558), AMP-514, pembrolizumab (MK-3475), REGN2810, PDR001, BGB-A317, or pidilizumab (CT-011)) is administered on day 15 of every 21 days. In some embodiments, the carboplatin is administered on day 1 of every 21 days. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the amount of ABRAXANE® administered to the individual is an effective amount described in Section V-C, infra. In some embodiments, the amount of ABRAXANE® administered to the individual is about 50 mg/m² to about 300 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the anti-PD-1 antibody is nivolumab. In some embodiments, about 0.01 to about 10 mg/kg of nivolumab is administered intravenously to the individual. In some embodiments, about 3 mg/kg of nivolumab is administered intravenously to the individual. In some embodiments, the nivolumab and nab-paclitaxel, e.g., ABRAXANE®, is administered to the individual at a dose or dose range described in Section V-C, infra. In some embodiments, the nivolumab and nab-paclitaxel, e.g., ABRAXANE®, is administered to the individual in a dosing schedule as described in Section V-C, infra. In some embodiments, the nab-paclitaxel, e.g., ABRAXANE®, is administered to the individual as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a dosing schedule as described in Section V-C, infra. In some embodiments, the nab-paclitaxel, e.g., ABRAXANE®, is administered to the individual as described hereinabove. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a dosing schedule as described in Section V-C, infra. In some embodiments, the nab-paclitaxel, e.g., ABRAXANE®, is administered to the individual as described hereinabove. In some embodiments, between about 0.01 mg/kg to about 10 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a dosing schedule as described in Section V-C, infra. In some embodiments, nab-paclitaxel, e.g., ABRAXANE®, is administered to the individual as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the lung cancer is NSCLC.

In some embodiments, there is provided a method of treating pancreatic cancer in an individual, comprising administering to said individual a) an effective amount of ABRAXANE®, and b) an effective amount of an antagonist of a PD-1 pathway in a cell. In some embodiments, there is provided a method of treating pancreatic cancer in an individual, comprising administering to said individual a) an effective amount of ABRAXANE®, and b) an effective amount of a PD-1 antagonist. In some embodiments, there is provided a method of treating pancreatic cancer in an individual, comprising administering to said individual a) an effective amount of ABRAXANE®, and b) an effective amount of an anti-PD-1 antibody (such as nivolumab (BMS-936558), AMP-514, pembrolizumab (MK-3475), REGN2810, PDR001, BGB-A317, or pidilizumab (CT-011)). In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the amount of ABRAXANE® administered to the individual is an effective amount described in Section V-C, infra. In some embodiments, the amount of ABRAXANE® administered to the individual is about 45 mg/m² to about 350 mg/m². In some embodiments, the amount of ABRAXANE® administered to the individual is about 50 mg/m² to about 300 mg/m². In some embodiments, the amount of ABRAXANE® administered to the individual is about 50 mg/m² to about 350 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, the anti-PD-1 antibody is nivolumab. In some embodiments, the nivolumab and nab-paclitaxel, e.g., ABRAXANE®, is administered to the individual at a dose or dose range described in Section V-C, infra. In some embodiments, the nivolumab and nab-paclitaxel, e.g., ABRAXANE®, is administered to the individual in a dosing schedule as described in Section V-C, infra. In some embodiments, the nivolumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a dosing schedule as described in Section V-C, infra. In some embodiments, AMP-514 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a dosing schedule as described in Section V-C, infra. In some embodiments, pembrolizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a dosing schedule as described in Section V-C, infra. In some embodiments, pidilizumab and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the REGN2810 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the PDR001 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the BGB-A317 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the method further comprises administering to the individual an effective amount of a nucleoside analog. In some embodiments, the nucleoside analog is gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, the nab-paclitaxel, e.g., ABRAXANE®, is administered intravenously. In some embodiments, the ABRAXANE® is administered intravenously. In some embodiments, the PD-1 pathway inhibitor is administered intravenously. In some embodiments, the antagonist of PD-1 is administered intravenously. In some embodiments, the anti-PD-1 antibody is administered intravenously. In some embodiments, the nab-paclitaxel, e.g., ABRAXANE®, and the anti-PD-1 antibody are administered intravenously. In some embodiments, the ABRAXANE® and the anti-PD-1 antibody are administered intravenously. In some embodiments, the nucleoside analog is administered intravenously. In some embodiments the gemcitabine is administered intravenously. In some embodiments, the nab-paclitaxel, e.g., ABRAXANE®, the anti-PD-1 antibody and the gemcitabine are administered intravenously.

In some embodiments, there is provided a method of treating pancreatic cancer in an individual, comprising: a) intravenously administering about 50 to about 350 mg/m² (such as about 75 mg/m², 100 mg/m², 125 mg/m², 150 mg/m², 200 mg/m², 225 mg/m², 250 mg/m²) of ABRAXANE® to the individual weekly, and b) intravenously administering about 0.01 to about 10 mg/kg (such as about 3 mg/kg) of an anti-PD-1 antibody (such as nivolumab (BMS-936558), AMP-514, pembrolizumab (MK-3475), REGN2810, PDR001, BGB-A317, or pidilizumab (CT-011)) to the individual twice out of every twenty eight days. In some embodiments, the method further comprises intravenously administering to the individual an effective amount of gemcitabine at the dose of 1000 mg/m² weekly. In some embodiments, the ABRAXANE® and the gemcitabine is administered on days 1, 8, and 15 of every 28 days and an anti-PD-1 antibody (such as nivolumab (BMS-936558), AMP-514, pembrolizumab (MK-3475), REGN2810, PDR001, BGB-A317, or pidilizumab (CT-011)) is administered on days 1 and 15 of every 28 days. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, the amount of ABRAXANE® administered to the individual is an effective amount described in Section V-C, infra. In some embodiments, the amount of ABRAXANE® administered to the individual is about 50 mg/m² to about 300 mg/m². In some embodiments, the amount of ABRAXANE® administered to the individual is about 60 mg/m² to about 300 mg/m². In some embodiments, the amount of ABRAXANE® administered to the individual is between about 75 mg/m² to about 275 mg/m². In some embodiments, the amount of ABRAXANE® administered to the individual is between about 80 mg/m² to about 350 mg/m². In some embodiments, the amount of ABRAXANE® administered to the individual is between about 80 mg/m² to about 300 mg/m². In some embodiments, the amount of ABRAXANE® administered to the individual is about 80 mg/m² to about 200 mg/m². In some embodiments, the amount of ABRAXANE® administered to the individual is between about 80 mg/m² to about 150 mg/m². In some embodiments, the amount of ABRAXANE® administered to the individual is between about 100 mg/m² to about 250 mg/m². In some embodiments, the amount of ABRAXANE® administered to the individual is between about 150 mg/m² to about 350 mg/m². In some embodiments, the amount of ABRAXANE® administered to the individual is between about 170 mg/m² to about 200 mg/m². In some embodiments, the amount of ABRAXANE® administered to the individual is between about 200 mg/m² to about 350 mg/m². In some embodiments, the amount of ABRAXANE® administered to the individual is about 50 mg/m². In some embodiments, the amount of ABRAXANE® administered to the individual is about 75 mg/m². In some embodiments, the amount of ABRAXANE® administered to the individual is about 100 mg/m². In some embodiments, the amount of ABRAXANE® administered to the individual is about 125 mg/m². In some embodiments, the amount of ABRAXANE® administered to the individual is about 150 mg/m². In some embodiments, the amount of ABRAXANE® administered to the individual is about 175 mg/m². In some embodiments, the amount of ABRAXANE® administered to the individual is about 200 mg/m². In some embodiments, the amount of ABRAXANE® administered to the individual is about 225 mg/m². In some embodiments, the amount of ABRAXANE® administered to the individual is about 250 mg/m². In some embodiments, the amount of ABRAXANE® administered to the individual is about 260 mg/m². In some embodiments, the amount of ABRAXANE® administered to the individual is about 275 mg/m². In some embodiments, the amount of ABRAXANE® administered to the individual is about 300 mg/m². In some embodiments, the amount of ABRAXANE® administered to the individual is about 325 mg/m². In some embodiments, the amount of ABRAXANE® administered to the individual is about 350 mg/m². In some embodiments, nab-paclitaxel is administered as described herein for ABRAXANE®. In some embodiments, In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, the anti-PD-1 antibody is nivolumab. In some embodiments, about 0.01 to about 10 mg/kg of nivolumab is administered intravenously to the individual. In some embodiments, about 3 mg/kg of nivolumab is administered intravenously to the individual. In some embodiments, the nivolumab and nab-paclitaxel, e.g., ABRAXANE®, is administered to the individual at a dose or dose range described in Section V-C, infra. In some embodiments, the nivolumab nab-paclitaxel, e.g., ABRAXANE®, is administered to the individual in a dosing schedule as described in Section V-C, infra. In some embodiments, the nab-paclitaxel, e.g., ABRAXANE®, is administered to the individual as described hereinabove. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 and nab-paclitaxel, e.g., ABRAXANE®, is administered to the individual at a dose or dose range described in Section V-C, infra. In some embodiments, AMP-514 and nab-paclitaxel, e.g., ABRAXANE®, is administered to the individual in a dosing schedule as described in Section V-C, infra. In some embodiments, nab-paclitaxel, e.g., ABRAXANE®, is administered to the individual as described hereinabove. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab. In some embodiments, pembrolizumab and nab-paclitaxel, e.g., ABRAXANE®, is administered to the individual at a dose or dose range described in Section V-C, infra. In some embodiments, pembrolizumab and nab-paclitaxel, e.g., ABRAXANE®, is administered to the individual in a dosing schedule as described in Section V-C, infra. In some embodiments, nab-paclitaxel, e.g., ABRAXANE®, is administered to the individual as described hereinabove. In some embodiments, between about 0.01 mg/kg to about 10 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab. In some embodiments, pidilizumab and nab-paclitaxel, e.g., ABRAXANE®, is administered to the individual at a dose or dose range described in Section V-C, infra. In some embodiments, pidilizumab and nab-paclitaxel, e.g., ABRAXANE®, is administered to the individual in a dosing schedule as described in Section V-C, infra. In some embodiments, nab-paclitaxel, e.g., ABRAXANE®, is administered to the individual as described hereinabove. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 and nab-paclitaxel, e.g., ABRAXANE®, is administered to the individual in a dosing schedule as described in Section V-C, infra. In some embodiments, the REGN2810 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 and nab-paclitaxel, e.g., ABRAXANE®, is administered to the individual in a dosing schedule as described in Section V-C, infra. In some embodiments, the PDR001 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 and nab-paclitaxel, e.g., ABRAXANE®, is administered to the individual in a dosing schedule as described in Section V-C, infra. In some embodiments, the BGB-A317 and nab-paclitaxel, e.g., ABRAXANE®, is administered to the individual at a dose or dose range described in Section V-C, infra. In some embodiments, the nab-paclitaxel, e.g., ABRAXANE®, is administered to the individual as described hereinabove. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the method further comprises administering to the individual an effective amount of a nucleoside analog. In some embodiments, the nucleoside analog is gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In some embodiments, there is provided a method of treating breast cancer in an individual comprising administering to said individual a) an effective amount of ABRAXANE®, and b) an effective amount of a PD-L1 antagonist. In some embodiments, there is provided a method of treating breast cancer comprising administering to said individual a) an effective amount of ABRAXANE®, and b) an effective amount of an anti-PD-L1 antibody (such as BMS-936559, MEDI4736, MSB0010718C and MPDL3280A). In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the anti-PD-L1 antibody is BMS-936559. In some embodiments, BMS-936559 and nab-paclitaxel, e.g., ABRAXANE®, is administered to the individual at a dose or dose range described in Section V-C, infra. In some embodiments, BMS-936559 and nab-paclitaxel, e.g., ABRAXANE®, is administered to the individual in a dosing schedule as described in Section V-C, infra. In some embodiments, BMS-936559 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the anti-PD-L1 antibody is MEDI4736. In some embodiments, MEDI4736 and nab-paclitaxel, e.g., ABRAXANE®, is administered to the individual at a dose or dose range described in Section V-C, infra. In some embodiments, MEDI4736 and nab-paclitaxel, e.g., ABRAXANE®, is administered to the individual in a dosing schedule as described in Section V-C, infra. In some embodiments, MEDI4736 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the anti-PD-L1 antibody is MPDL3280A. In some embodiments, MPDL3280A and nab-paclitaxel, e.g., ABRAXANE®, is administered to the individual at a dose or dose range described in Section V-C, infra. In some embodiments, MPDL3280A and nab-paclitaxel, e.g., ABRAXANE®, is administered to the individual in a dosing schedule as described in Section V-C, infra. In some embodiments, MPDL3280A and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the anti-PD-L1 antibody is MSB0010718C. In some embodiments, MSB0010718C and nab-paclitaxel, e.g., ABRAXANE®, is administered to the individual at a dose or dose range described in Section V-C, infra. In some embodiments, MSB0010718C and nab-paclitaxel, e.g., ABRAXANE®, is administered to the individual in a dosing schedule as described in Section V-C, infra. In some embodiments, MSB0010718C and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the breast cancer is a Her2 negative breast cancer. In some embodiments, the breast cancer is triple negative breast cancer. In some embodiments, the breast cancer is a metastatic breast cancer.

In some embodiments, there is provided a method of treating lung cancer in an individual, comprising administering to said individual a) an effective amount of ABRAXANE®, and b) an effective amount of a PD-L1 antagonist. In some embodiments, there is provided a method of treating lung cancer in an individual, comprising administering to said individual a) an effective amount of ABRAXANE®, and b) an effective amount of an anti-PD-L1 antibody (such as BMS-936559, MEDI4736, MSB0010718C and MPDL3280A). In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, the nab-paclitaxel (e.g., ABRAXANE®) is administered at least once during a cycle. In some embodiments, nab-paclitaxel is administered as described herein for ABRAXANE®. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the anti-PD-L1 antibody is administered via intravenous administration. In some embodiments, the anti-PD-L1 antibody is administered via intra-arterial administration. In some embodiments, the anti-PD-L1 antibody is administered via intraperitoneal administration. In some embodiments, the anti-PD-L1 antibody is administered via intrapulmonary administration. In some embodiments, the anti-PD-L1 antibody is administered via oral administration. In some embodiments, the anti-PD-L1 antibody is administered via inhalation. In some embodiments, the anti-PD-L1 antibody is administered via intravesicular administration. In some embodiments, the anti-PD-L1 antibody is administered via intramuscular administration. In some embodiments, the anti-PD-L1 antibody is administered via intra-tracheal administration. In some embodiments, the anti-PD-L1 antibody is administered via subcutaneous administration. In some embodiments, the anti-PD-L1 antibody is administered via intraocular administration. In some embodiments, the anti-PD-L1 antibody is administered via intrathecal administration. In some embodiments, the anti-PD-L1 antibody is administered via transmucosal administration. In some embodiments, the anti-PD-L1 antibody is administered via transdermal administration. In some embodiments, the anti-PD-L1 antibody is administered as a sustained continuous release formulation. In some embodiments, the anti-PD-L1 antibody is administered via an inhaler or other air borne delivery system. In some embodiments, the anti-PD-L1 antibody is BMS-936559. In some embodiments, BMS-936559 and nab-paclitaxel, e.g. ABRAXANE®, is administered to the individual at a dose or dose range described in Section V-C, infra. In some embodiments, BMS-936559 and nab-paclitaxel, e.g. ABRAXANE®, is administered to the individual in a dosing schedule as described in Section V-C, infra. In some embodiments, BMS-936559 and nab-paclitaxel, e.g. ABRAXANE®, is administered to the individual in a mode of administration described in Section V-C, infra. In some embodiments, nab-paclitaxel, e.g. ABRAXANE®, is administered as described hereinabove. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the anti-PD-L1 antibody is MEDI4736. In some embodiments, MEDI4736 and nab-paclitaxel, e.g. ABRAXANE®, is administered to the individual at a dose or dose range described in Section V-C, infra. In some embodiments, MEDI4736 and nab-paclitaxel, e.g. ABRAXANE®, is administered to the individual in a dosing schedule as described in Section V-C, infra. In some embodiments, MEDI4736 and nab-paclitaxel, e.g. ABRAXANE®, is administered to the individual in a mode of administration described in Section V-C, infra. In some embodiments, the nab-paclitaxel, e.g. ABRAXANE®, is administered as described hereinabove. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the anti-PD-L1 antibody is MPDL3280A. In some embodiments, MPDL3280A and nab-paclitaxel, e.g. ABRAXANE®, is administered to the individual at a dose or dose range described in Section V-C, infra. In some embodiments, MPDL3280A and nab-paclitaxel, e.g. ABRAXANE®, is administered to the individual in a dosing schedule as described in Section V-C, infra. In some embodiments, MPDL3280A and nab-paclitaxel, e.g. ABRAXANE®, is administered to the individual in a mode of administration described in Section V-C, infra. In some embodiments, the nab-paclitaxel, e.g. ABRAXANE® is administered as described hereinabove. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the anti-PD-L1 antibody is MSB0010718C. In some embodiments, MSB0010718C and nab-paclitaxel, e.g. ABRAXANE®, is administered to the individual at a dose or dose range described in Section V-C, infra. In some embodiments, MSB0010718C and nab-paclitaxel, e.g. ABRAXANE®, is administered to the individual in a dosing schedule as described in Section V-C, infra. In some embodiments, MSB0010718C and nab-paclitaxel, e.g. ABRAXANE®, is administered to the individual in a mode of administration described in Section V-C, infra. In some embodiments, the nab-paclitaxel, e.g. ABRAXANE®, is administered as described hereinabove. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the method further comprises administering to the individual an effective amount of a platinum-based agent. In some embodiments, the platinum-based agent is carboplatin. In some embodiments, the platinum-based agent is cisplatin. In some embodiments, the lung cancer is NSCLC.

In some embodiments, there is provided a method of treating pancreatic cancer in an individual, comprising administering to said individual a) an effective amount of ABRAXANE®, and b) an effective amount of a PD-L1 antagonist. In some embodiments, there is provided a method of treating pancreatic cancer in an individual, comprising administering to said individual a) an effective amount of ABRAXANE®, and b) an effective amount of an anti-PD-L1 antibody (such as BMS-936559, MEDI4736, MSB0010718C and MPDL3280A). In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the anti-PD-L1 antibody is administered via intravenous administration. In some embodiments, the anti-PD-L1 antibody is administered via intra-arterial administration. In some embodiments, the anti-PD-L1 antibody is administered via intraperitoneal administration. In some embodiments, the anti-PD-L1 antibody is administered via intrapulmonary administration. In some embodiments, the anti-PD-L1 antibody is administered via oral administration. In some embodiments, the anti-PD-L1 antibody is administered via inhalation. In some embodiments, the anti-PD-L1 antibody is administered via intravesicular administration. In some embodiments, the anti-PD-L1 antibody is administered via intramuscular administration. In some embodiments, the anti-PD-L1 antibody is administered via intra-tracheal administration. In some embodiments, the anti-PD-L1 antibody is administered via subcutaneous administration. In some embodiments, the anti-PD-L1 antibody is administered via intraocular administration. In some embodiments, the anti-PD-L1 antibody is administered via intrathecal administration. In some embodiments, the anti-PD-L1 antibody is administered via transmucosal administration. In some embodiments, the anti-PD-L1 antibody is administered via transdermal administration. In some embodiments, the anti-PD-L1 antibody is administered as a sustained continuous release formulation. In some embodiments, the anti-PD-L1 antibody is administered via an inhaler or other air borne delivery system. In some embodiments, the anti-PD-L1 antibody is BMS-936559. In some embodiments, BMS-936559 and nab-paclitaxel, e.g., ABRAXANE®, is administered to the individual at a dose or dose range described in Section V-C, infra. In some embodiments, BMS-936559 and nab-paclitaxel, e.g., ABRAXANE®, is administered to the individual in a dosing schedule as described in Section V-C, infra. In some embodiments, BMS-936559 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the anti-PD-L1 antibody is MEDI4736. In some embodiments, MEDI4736 and nab-paclitaxel, e.g., ABRAXANE®, is administered to the individual at a dose or dose range described in Section V-C, infra. In some embodiments, MEDI4736 and nab-paclitaxel, e.g., ABRAXANE®, is administered to the individual in a dosing schedule as described in Section V-C, infra. In some embodiments, MEDI4736 and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the anti-PD-L1 antibody is MPDL3280A. In some embodiments, MPDL3280A and nab-paclitaxel, e.g., ABRAXANE®, is administered to the individual at a dose or dose range described in Section V-C, infra. In some embodiments, MPDL3280A and nab-paclitaxel, e.g., ABRAXANE®, is administered to the individual in a dosing schedule as described in Section V-C, infra. In some embodiments, MPDL3280A and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the anti-PD-L1 antibody is MSB0010718C. In some embodiments, MSB0010718C and nab-paclitaxel, e.g., ABRAXANE®, is administered to the individual at a dose or dose range described in Section V-C, infra. In some embodiments, MSB0010718C and nab-paclitaxel, e.g., ABRAXANE®, is administered to the individual in a dosing schedule as described in Section V-C, infra. In some embodiments, MSB0010718C and nab-paclitaxel, e.g., ABRAXANE®, is administered to the individual in a mode of administration described in Section V-C, infra. In some embodiments, the nab-paclitaxel, e.g., ABRAXANE®, is administered as described hereinabove. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the method further comprises administering to the individual an effective amount of a nucleoside analog. In some embodiments, the nucleoside analog is gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In some embodiments, there is provided a method of treating breast cancer in an individual, comprising administering to said individual a) an effective amount of ABRAXANE®, and b) an effective amount of a PD-L2 antagonist. In some embodiments, there is provided a method of treating breast cancer in an individual, comprising administering to said individual a) an effective amount of ABRAXANE®, and b) an effective amount of an anti-PD-L2 antibody (for example, rHIgM12B7). In some embodiments, the anti-PDL2 antibody is rHIgM12B7. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the breast cancer is a Her2 negative breast cancer. In some embodiments, the breast cancer is a triple negative breast cancer. In some embodiments, the breast cancer is a metastatic breast cancer.

In some embodiments, there is provided a method of treating lung cancer in an individual, comprising administering to said individual a) an effective amount of ABRAXANE®, and b) an effective amount of a PD-L2 antagonist. In some embodiments, there is provided a method of treating pancreatic cancer in an individual, comprising administering to said individual a) an effective amount of ABRAXANE®, and b) an effective amount of an anti-PD-L2 antibody, such as rHIgM12B7. In some embodiments, the anti-PD-L2 antibody is rHIgM12B7. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the method further comprises administering to the individual an effective amount of a platinum-based agent. In some embodiments, the platinum-based agent is carboplatin. In some embodiments, the platinum-based agent is cisplatin. In some embodiments, the lung cancer is NSCLC.

In some embodiments, there is provided a method of treating pancreatic cancer in an individual, comprising administering to said individual a) an effective amount of ABRAXANE®, and b) an effective amount of a PD-L2 antagonist. In some embodiments, there is provided a method of treating pancreatic cancer in an individual, comprising administering to said individual a) an effective amount of ABRAXANE®, and b) an effective amount of an anti-PD-L2 antibody, such as rHIgM12B7. In some embodiments, the anti-PD-L2 antibody is rHIgM12B7. In some embodiments, ABRAXANE® is administered to the individual at a dose or dose range described in Section V-C, infra. In some embodiments, ABRAXANE® is administered to the individual in a dosing schedule as described in Section V-C, infra. In some embodiments, ABRAXANE® is administered to the individual in a mode of administration described in Section V-C, infra. In some embodiments, the effective amount of ABRAXANE® is an effective amount described in Section V-C, infra. In some embodiments, the effective amount of ABRAXANE® is between about 45 mg/m² to about 350 mg/m². In some embodiments, the effective amount of ABRAXANE® is about 50 mg/m² to about 300 mg/m². In some embodiments, the effective amount of ABRAXANE® is about 50 mg/m² to about 350 mg/m². In some embodiments, the effective amount of ABRAXANE® is about 60 mg/m² to about 300 mg/m². In some embodiments, the effective amount of ABRAXANE® is between about 75 mg/m² to about 275 mg/m². In some embodiments, the effective amount of ABRAXANE® is between about 80 mg/m² to about 350 mg/m². In some embodiments, the effective amount of ABRAXANE® is between about 80 mg/m² to about 300 mg/m². In some embodiments, the effective amount of ABRAXANE® is about 80 mg/m² to about 200 mg/m². In some embodiments, the effective amount of ABRAXANE® is between about 80 mg/m² to about 150 mg/m². In some embodiments, the effective amount of ABRAXANE® is between about 100 mg/m² to about 250 mg/m². In some embodiments, the effective amount of ABRAXANE® is between about 150 mg/m² to about 350 mg/m². In some embodiments, the effective amount of ABRAXANE® is between about 170 mg/m² to about 200 mg/m². In some embodiments, the effective amount of ABRAXANE® is between about 200 mg/m² to about 350 mg/m². In some embodiments, the effective amount of ABRAXANE® is about 50 mg/m². In some embodiments, the effective amount of ABRAXANE® is about 75 mg/m². In some embodiments, the effective amount of ABRAXANE® is about 100 mg/m². In some embodiments, the effective amount of ABRAXANE® is about 125 mg/m². In some embodiments, the effective amount of ABRAXANE® is about 150 mg/m². In some embodiments, the effective amount of ABRAXANE® is about 175 mg/m². In some embodiments, the effective amount of ABRAXANE® is about 200 mg/m². In some embodiments, the effective amount of ABRAXANE® is about 225 mg/m². In some embodiments, the effective amount of ABRAXANE® is about 250 mg/m². In some embodiments, the effective amount of ABRAXANE® is about 260 mg/m². In some embodiments, the effective amount of ABRAXANE® is about 275 mg/m². In some embodiments, the effective amount of ABRAXANE® is about 300 mg/m². In some embodiments, the effective amount of ABRAXANE® is about 325 mg/m². In some embodiments, the effective amount of ABRAXANE® is about 350 mg/m². In some embodiments, nab-paclitaxel is administered as described herein for ABRAXANE®. In some embodiments, the nab-paclitaxel (e.g., ABRAXANE®) is administered at least once during a cycle. In some embodiments, the nab-paclitaxel (e.g., ABRAXANE®) is administered at least twice during a cycle. In some embodiments, the nab-paclitaxel (e.g., ABRAXANE®) is administered at least three times during a cycle. In some embodiments, the nab-paclitaxel (e.g., ABRAXANE®) is administered at least once a week. In some embodiments, the nab-paclitaxel (e.g., ABRAXANE®) is administered at least once every two weeks. In some embodiments, the nab-paclitaxel (e.g., ABRAXANE®) is administered at least once every three weeks. In some embodiments, the nab-paclitaxel (e.g., ABRAXANE®) is administered at least once every four weeks. In some embodiments, a cycle is 21 days. In some embodiments, a cycle is 28 days. In some embodiments, the nab-paclitaxel (e.g., ABRAXANE®) is administered on days 1, 8 and 15 of a 28 day cycle. In some embodiments, the nab-paclitaxel (e.g., ABRAXANE®) is administered on day 1 of a 21 day cycle. In some embodiments, the nab-paclitaxel (e.g., ABRAXANE®) is administered on days 1, 8 and 15 of a 21 day cycle. In some embodiments, ABRAXANE® is administered via intravenous administration. In some embodiments, ABRAXANE® is administered via intra-arterial administration. In some embodiments, ABRAXANE® is administered via intraperitoneal administration. In some embodiments, ABRAXANE® is administered via intrapulmonary administration. In some embodiments, ABRAXANE® is administered via oral administration. In some embodiments, ABRAXANE® is administered via inhalation. In some embodiments, ABRAXANE® is administered via intravesicular administration. In some embodiments, ABRAXANE® is administered via intramuscular administration. In some embodiments, ABRAXANE® is administered via intra-tracheal administration. In some embodiments, ABRAXANE® is administered via subcutaneous administration. In some embodiments, ABRAXANE® is administered via intraocular administration. In some embodiments, ABRAXANE® is administered via intrathecal administration. In some embodiments, ABRAXANE® is administered via transmucosal administration. In some embodiments, ABRAXANE® is administered via transdermal administration. In some embodiments, the effective amount of ABRAXANE® is administered as a sustained continuous release formulation. In some embodiments, ABRAXANE® is administered via an inhaler or other air borne delivery system. In some embodiments, the method further comprises administering to the individual an effective amount of a nucleoside analog. In some embodiments, the nucleoside analog is gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

Also provided are kits and compositions useful for the methods described herein.

### IV. BRIEF DESCRIPTION OF FIGURES

FIG. 1 shows the study design for the treatment with nab-paclitaxel and nivolumab in patients with Her2 negative recurrent metastatic breast cancer after one prior regimen for metastatic disease, including an anthracycline unless clinically contraindicated (mBC Arms E and F, Parts 1 and 2).

FIG. 2 shows the study design for the treatment with nab-paclitaxel and nivolumab with or without gemcitabine in patients with adenocarcinoma of the pancreas with 1 prior systemic chemotherapy (Panc Arm a dose-limiting toxicity assessment, Part 1 only); or no prior chemotherapy, surgery or radiation therapy for locally advanced or metastatic disease (Panc Arm A, Part 2 and Panc Arm B).

FIG. 3 shows the study design for the treatment with nab-paclitaxel, nivolumab, and carboplatin in patients with Stage IIIB or IV NSCLC with no prior chemotherapy for metastatic disease and in subjects who are not candidates for curative surgery or radiation (NSCLC Arms C and D, Parts 1 and 2).

### V. DETAILED DESCRIPTION

In one aspect, provided herein are methods of combination therapy comprising a first agent comprising administration of a composition comprising nanoparticles comprising a taxane (such as paclitaxel) and a carrier protein (such as albumin) in conjunction with a second agent that antagonizes a PD-1 pathway in a cell (also referred to as an “PD-1 pathway antagonist,” or “PD-1 pathway inhibitor”). In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the nanoparticle composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 100 mg/m² to about 150 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the second agent is referred to as “the other agent.” In some embodiments, the other agent is an agent described in Section V-C, infra. In some embodiments, the second agent is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the second agent is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the second agent is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the second agent is AMP-514. In some embodiments, the second agent is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the second agent is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the second agent is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the second agent is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the second agent is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the second agent is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle. In some embodiments, the other agent is administered in a mode of administration described in Section V-C, infra. In some embodiments, the second agent is administered via inhalation. In some embodiments, the second agent is administered via intravesicular administration. In some embodiments, the second agent is administered via intramuscular administration. In some embodiments, the second agent is administered via intra-tracheal administration. In some embodiments, the second agent is administered via subcutaneous administration. In some embodiments, the second agent is administered via intraocular administration. In some embodiments, the second agent is administered via intrathecal administration. In some embodiments, the second agent is administered via transmucosal administration. In some embodiments, the second agent is administered via transdermal administration. In some embodiments, the second agent is administered as a sustained continuous release formulation. In some embodiments, the second agent is administered via an inhaler or other air borne delivery system. In some embodiments, the other agent is administered at a dosing frequency described in Section V-C, infra. In some embodiments, the other agent is administered in a dosing schedule as described in Section V-C, infra. In some embodiments, the other agent is administered in a dose or dose range described in Section V-C, infra. In some embodiments, the nanoparticle comprises the first agent and the second agent. In some embodiments, the second agent is administered in a nanoparticle. In some embodiments, the second agent is administered in the same nanoparticle as the first agent. In some embodiments, the second agent is administered in a different nanoparticle from the nanoparticle of the first agent.

The present application thus provides methods of combination therapy. It is to be understood by a person of ordinary skill in the art that the combination therapy methods described herein requires that one agent or composition be administered in conjunction with another agent. “In conjunction with” refers to administration of one treatment modality in addition to another treatment modality, such as administration of a nanoparticle composition described herein in addition to administration of the other agent to the same individual. As such, “in conjunction with” refers to administration of one treatment modality before, during or after delivery of the other treatment modality to the individual.

The methods described herein are generally useful for treatment of proliferative diseases. As used herein, “treatment” is an approach for obtaining beneficial or desired clinical results, and beneficial or desired clinical results can include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods provided herein contemplate any one or more of these aspects of treatment.

An “agent that antagonizes a programmed death-1 or programmed cell death-1 pathway,” “agent that antagonizes a programmed death-1 or programmed cell death-1/PCD-1 pathway,” “agent that antagonizes a PD-1 pathway,” “PD-1 pathway antagonist,” or “PD-1 pathway inhibitor” refers to an agent that antagonizes the activity of a component of the PD-1 pathway or interferes with the interaction of the components of the PD-1 pathway. For example, in some embodiments, the PD-1 pathway antagonist antagonizes the activity of PD-1 or a ligand of PD-1 (such as PD-L1, or PD-L2). In some embodiments, the PD-1 pathway antagonist disrupts or interferes with the interaction of the components of the PD-1 pathway. For example, in some embodiments, the PD-1 pathway antagonist interferes with the interaction between PD-1 and a ligand of PD-1. In some embodiments, the PD-1 pathway antagonist interferes with the interaction between PD-1 and PD-L1. In some embodiments, the PD-1 pathway antagonist interferes with the interaction between PD-1 and PD-L2. In some embodiments, a PD-1 pathway antagonist comprises a protein, antibody, small molecule, fusion protein, or a combination thereof.

An “agent that antagonizes PD-1,” “PD-1 antagonist,” or “PD-1 inhibitor” refers to an agent that antagonizes the activity of PD-1 or interferes with the interaction between PD-1 and a ligand of PD-1. For example, in some embodiments, the PD-1 antagonist antagonizes the activity of PD-1. In some embodiments, the PD-1 antagonist disrupts or interferes with the interaction between PD-1 and a ligand of PD-1. In some embodiments, the PD-1 antagonist interferes with the interaction between PD-1 and PD-L1. In some embodiments, the PD-1 antagonist interferes with the interaction between PD-1 and PD-L2. In some embodiments, a PD-1 antagonist comprises a protein, antibody, small molecule, fusion protein, or a combination thereof.

An “agent that antagonizes PD-L1,” “PD-L1 antagonist,” or “PD-L1 inhibitor” refers to an agent that antagonizes the activity of PD-L1 or interferes with the interaction between PD-L1 and another component of the PD-1 pathway. For example, in some embodiments, the PD-L1 antagonist antagonizes the activity of PD-L1. In some embodiments, the PD-1 antagonist disrupts or interferes with the interaction between PD-L1 and another component of the PD-1 pathway. In some embodiments, the PD-L1 antagonist interferes with the interaction between PD-L1 and PD-1. In some embodiments, a PD-L1 antagonist comprises a protein, antibody, small molecule, fusion protein, or a combination thereof.

An “agent that antagonizes PD-L2,” “PD-L2 antagonist,” or “PD-L2 inhibitor” refers to an agent that antagonizes the activity of PD-L2 or interferes with the interaction between PD-L2 and another component of the PD-1 pathway. For example, in some embodiments, the PD-L2 antagonist antagonizes the activity of PD-L2. In some embodiments, the PD-1 antagonist disrupts or interferes with the interaction between PD-L2 and another component of the PD-1 pathway. In some embodiments, the PD-L2 antagonist interferes with the interaction between PD-L2 and PD-1. In some embodiments, a PD-L2 antagonist comprises a protein, antibody, small molecule, fusion protein, or a combination thereof.

Individuals having “Her2 negative breast cancer” used herein refer to individuals who are clinically negative for expression of HER2 protein.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation.

The term “individual” is a mammal, including humans. An individual includes, but is not limited to, human, bovine, horse, feline, canine, rodent, or primate. In some embodiments, the individual is human.

The methods may be practiced in an adjuvant setting. “Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

The methods provided herein may also be practiced in a “neoadjuvant setting,” i.e., the method may be carried out before the primary/definitive therapy. In some embodiments, the individual has previously been treated. In some embodiments, the individual has not previously been treated. In some embodiments, the treatment is a first line therapy.

It is understood that aspect and embodiments described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations described herein include “consisting” and/or “consisting essentially of” aspects and variations.

### Section V-A. Methods of Combination Therapy

In one aspect, provided herein are methods of treating a proliferative disease (such as cancer) in an individual, comprising administering to the individual: a) an effective amount of a composition comprising nanoparticles comprising a taxane (such as paclitaxel) and a carrier protein (such as albumin); and b) an effective amount of at least one other agent that antagonizes a PD-1 pathway in a cell. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the amount of the taxane (such as paclitaxel) in the nanoparticle composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the other agent is administered in a mode of administration described in Section V-C, infra. In some embodiments, the other agent is administered via intravenous administration. In some embodiments, the other agent is administered via intra-arterial administration. In some embodiments, the other agent is administered via intraperitoneal administration. In some embodiments, the other agent is administered via intrapulmonary administration. In some embodiments, the other agent is administered via oral administration. In some embodiments, the other agent is administered via inhalation. In some embodiments, the other agent is administered via intravesicular administration. In some embodiments, the other agent is administered via intramuscular administration. In some embodiments, the other agent is administered via intra-tracheal administration. In some embodiments, the other agent is administered via subcutaneous administration. In some embodiments, the other agent is administered via intraocular administration. In some embodiments, the other agent is administered via intrathecal administration. In some embodiments, the other agent is administered via transmucosal administration. In some embodiments, the other agent is administered via transdermal administration. In some embodiments, the other agent is administered as a sustained continuous release formulation. In some embodiments, the other agent is administered via an inhaler or other air borne delivery system. In some embodiments, the other agent is administered at a dosing frequency described in Section V-C, infra. In some embodiments, the other agent is administered in a dosing schedule as described in Section V-C, infra. In some embodiments, the other agent is administered at a dose or dose range described in Section V-C, infra. In some embodiments, the other agent that antagonizes a PD-1 pathway is an antagonist of a component of the PD-1 pathway. In some embodiments, the other agent antagonizes PD-1, a ligand of PD-1 (such as PD-L1 or PD-L2), or a combination thereof. In some embodiments, the other agent is selected from the group consisting of AMP-224, nivolumab, AMP-514, pembrolizumab, pidilizumab, REGN2810, PDR001, BGB-A317, BMS-936559, MEDI4736, MSB0010718C, MPDL3280A and rHIgM12B7. In some embodiments, the other agent is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the other agent is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the other agent is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the other agent is AMP-514. In some embodiments, the other agent is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the other agent is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the other agent is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the other agent is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the other agent is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the other agent is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin); and b) an effective amount of at least one other agent that antagonizes a PD-1 pathway in a cell, wherein the nanoparticle composition and the other agent are administered concurrently. In some embodiments, the administration of the nanoparticle composition and the other agent are initiated at about the same time (for example, within any one of 1, 2, 3, 4, 5, 6, or 7 days). In some embodiments, the administrations of the nanoparticle composition and the other agent are terminated at about the same time (for example, within any one of 1, 2, 3, 4, 5, 6, or 7 days). In some embodiments, the administration of the other agent continues (for example for about any one of 0.5, 1, 2, 3, 4, 5, 6, 7, 8, 9, 10, 11, or 12 months) after the termination of the administration of the nanoparticle composition. In some embodiments, the administration of the other agent is initiated after (for example after about any one of 0.5, 1, 2, 3, 4, 5, 6, 7, 8, 9, 10, 11, or 12 months) the initiation of the administration of the nanoparticle composition. In some embodiments, the administrations of the nanoparticle composition and the other agent are initiated and terminated at about the same time. In some embodiments, the administrations of the nanoparticle composition and the other agent are initiated at about the same time and the administration of the other agent continues (for example for about any one of 0.5, 1, 2, 3, 4, 5, 6, 7, 8, 9, 10, 11, or 12 months) after the termination of the administration of the nanoparticle composition. In some embodiments, the administration of the nanoparticle composition and the other agent stop at about the same time and the administration of the other agent is initiated after (for example after about any one of 0.5, 1, 2, 3, 4, 5, 6, 7, 8, 9, 10, 11, or 12 months) the initiation of the administration of the nanoparticle composition. In some embodiments, the administration of the nanoparticle composition and the other agent stop at about the same time and the administration of the nanoparticle composition is initiated after (for example after about any one of 0.5, 1, 2, 3, 4, 5, 6, 7, 8, 9, 10, 11, or 12 months) the initiation of the administration of the other agent. In some embodiments, the proliferative disease is a cancer described in Section V-B, infra. In some embodiments, the cancer is breast cancer. In some embodiments, the breast cancer is a breast cancer described in Section V-B, infra. In some embodiments, the breast cancer is HER2 negative. In some embodiments, the breast cancer is ER negative. In some embodiments, the breast cancer is PR negative. In some embodiments, the breast cancer is HER2 negative and ER negative. In some embodiments, the breast cancer is HER2 negative and PR negative. In some embodiments, the breast cancer is ER negative and PR negative. In some embodiments, the breast cancer is HER2 negative, ER negative and PR negative. In some embodiments, the breast cancer is Her2 negative breast cancer, metastatic breast cancer, recurrent breast cancer, or a combination thereof. In some embodiments, the cancer is lung cancer. In some embodiments, the lung cancer is a lung cancer described in Section V-B, infra. In some embodiments, the lung cancer is NSCLC. In some embodiments, the cancer is pancreatic cancer. In some embodiments, the pancreatic cancer is a pancreatic cancer described in Section V-B, infra. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the amount of the taxane (such as paclitaxel) in the nanoparticle composition is an amount described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the other agent is administered in a mode of administration described in Section V-C, infra. In some embodiments, the other agent is administered via intravenous administration. In some embodiments, the other agent is administered via intra-arterial administration. In some embodiments, the other agent is administered via intraperitoneal administration. In some embodiments, the other agent is administered via intrapulmonary administration. In some embodiments, the other agent is administered via oral administration. In some embodiments, the other agent is administered via inhalation. In some embodiments, the other agent is administered via intravesicular administration. In some embodiments, the other agent is administered via intramuscular administration. In some embodiments, the other agent is administered via intra-tracheal administration. In some embodiments, the other agent is administered via subcutaneous administration. In some embodiments, the other agent is administered via intraocular administration. In some embodiments, the other agent is administered via intrathecal administration. In some embodiments, the other agent is administered via transmucosal administration. In some embodiments, the other agent is administered via transdermal administration. In some embodiments, the other agent is administered as a sustained continuous release formulation. In some embodiments, the other agent is administered via an inhaler or other air borne delivery system. In some embodiments, the other agent is administered at a dosing frequency described in Section V-C, infra. In some embodiments, the other agent is administered in a dosing schedule as described in Section V-C, infra. In some embodiments, the other agent is administered at a dose or dose range described in Section V-C, infra. In some embodiments, the other agent that antagonizes a PD-1 pathway is an antagonist of a component of the PD-1 pathway. In some embodiments, the other agent antagonizes PD-1, a ligand of PD-1 (such as PD-L1 or PD-L2), or a combination thereof. In some embodiments, the other agent is selected from the group consisting of AMP-224, nivolumab, AMP-514, pembrolizumab, pidilizumab, REGN2810, PDR001, BGB-A317, BMS-936559, MEDI4736, MSB0010718C, MPDL3280A and rHIgM12B7. In some embodiments, the other agent is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the other agent is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the other agent is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the other agent is AMP-514. In some embodiments, the other agent is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the other agent is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the other agent is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the other agent is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the other agent is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the other agent is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, the taxane is any of (and in some embodiments consisting essentially of) paclitaxel, tesetaxel, docetaxel, and ortataxel. In some embodiments, the taxane is paclitaxel. In some embodiments, the taxane is docetaxel. In some embodiments, the nanoparticle composition comprises ABRAXANE®. In some embodiments, the nanoparticle composition is ABRAXANE®.

In some embodiments, there is provided a method of treating a proliferative disease (such as cancer) in an individual, comprising administering to the individual: a) an effective amount of a composition comprising nanoparticles comprising a taxane (such as paclitaxel) coated with a carrier protein (such as albumin); and b) an effective amount of at least one other agent that antagonizes a PD-1 pathway in a cell. In some embodiments, the nanoparticles have an average size of 20-400 nm, such as 40-200 nm. In some embodiments, there is provided a method of treating a proliferative disease (such as cancer) in an individual, comprising administering to the individual: a) an effective amount of ABRAXANE®; and b) an effective amount of at least one other agent that antagonizes a PD-1 pathway in a cell. In some embodiments, the nanoparticle composition (such as ABRAXANE®) and the other agent are administered concurrently. In some embodiments, the proliferative disease is a cancer selected from the group consisting of breast cancer, lung cancer (such as small cell lung cancer and non-small cell lung cancer), and pancreatic cancer. In some embodiments, the proliferative disease is a cancer described in Section V-B, infra. In some embodiments, the cancer is breast cancer. In some embodiments, the breast cancer is a breast cancer described in Section V-B, infra. In some embodiments, the breast cancer is HER2 negative. In some embodiments, the breast cancer is ER negative. In some embodiments, the breast cancer is PR negative. In some embodiments, the breast cancer is HER2 negative and ER negative. In some embodiments, the breast cancer is HER2 negative and PR negative. In some embodiments, the breast cancer is ER negative and PR negative. In some embodiments, the breast cancer is HER2 negative, ER negative and PR negative. In some embodiments, the breast cancer is Her2 negative breast cancer, metastatic breast cancer, recurrent breast cancer, or a combination thereof. In some embodiments, the cancer is lung cancer. In some embodiments, the lung cancer is a lung cancer described in Section V-B, infra. In some embodiments, the lung cancer is NSCLC. In some embodiments, the cancer is pancreatic cancer. In some embodiments, the pancreatic cancer is a pancreatic cancer described in Section V-B, infra. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the nanoparticle composition is an amount described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the effective amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the other agent is administered in a mode of administration described in Section V-C, infra. In some embodiments, the other agent is administered via intravenous administration. In some embodiments, the other agent is administered via intra-arterial administration. In some embodiments, the other agent is administered via intraperitoneal administration. In some embodiments, the other agent is administered via intrapulmonary administration. In some embodiments, the other agent is administered via oral administration. In some embodiments, the other agent is administered via inhalation. In some embodiments, the other agent is administered via intravesicular administration. In some embodiments, the other agent is administered via intramuscular administration. In some embodiments, the other agent is administered via intra-tracheal administration. In some embodiments, the other agent is administered via subcutaneous administration. In some embodiments, the other agent is administered via intraocular administration. In some embodiments, the other agent is administered via intrathecal administration. In some embodiments, the other agent is administered via transmucosal administration. In some embodiments, the other agent is administered via transdermal administration. In some embodiments, the other agent is administered as a sustained continuous release formulation. In some embodiments, the other agent is administered via an inhaler or other air borne delivery system. In some embodiments, the other agent is administered at a dosing frequency described in Section V-C, infra. In some embodiments, the other agent is administered in a dosing schedule as described in Section V-C, infra. In some embodiments, the other agent is administered at a dose or dose range described in Section V-C, infra. In some embodiments, the other agent that antagonizes a PD-1 pathway is an antagonist of a component of the PD-1 pathway. In some embodiments, the other agent antagonizes PD-1, a ligand of PD-1 (such as PD-L1 or PD-L2), or a combination thereof. In some embodiments, the other agent is selected from the group consisting of AMP-224, nivolumab, AMP-514, pembrolizumab, pidilizumab, REGN2810, PDR001, BGB-A317, BMS-936559, MEDI4736, MSB0010718C, MPDL3280A and rHIgM12B7. In some embodiments, the other agent is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the other agent is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the other agent is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the other agent is AMP-514. In some embodiments, the other agent is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the other agent is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the other agent is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the other agent is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the other agent is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the other agent is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, the other agent that antagonizes a PD-1 pathway is an antibody. In some embodiments, the other agent that antagonizes a PD-1 pathway is a monoclonal antibody. In some embodiments, the other agent that antagonizes a PD-1 pathway is human antibody. In some embodiments, a human antibody is an antibody that is based on or derived from a human. In some embodiments, the other agent that antagonizes a PD-1 pathway is a fully human antibody. In some embodiments, a fully human antibody comprises an antibody consisting essentially of an antibody or antibody fragments that are derived from or based from a human antibody. In some embodiments, the other agent that antagonizes a PD-1 pathway is a humanized antibody. In some embodiments, a humanized antibody comprises an antibody comprising one or more human-derived antibody regions or fragments. In some embodiments, the other agent that antagonizes a PD-1 pathway is a chimeric antibody. In some embodiments, the chimeric antibody comprises antibody fragments that are derived from or based on two or more antibodies from two or more species. In some embodiments, the chimeric antibody comprises antibody fragments that are derived from or based on two or more antibodies from two or more subjects. In some embodiments, the two or more subjects are of the same species. In some embodiments, the two or more subjects are of different species. In some embodiments, the other agent comprises at least a portion of an antibody. In some embodiments, the other agent comprises at least a portion of an immunoglobulin G (IgG), immunoglobulin M (IgM), immunoglobulin E (IgE), immunoglobulin D (IgD), immunoglobulin A (IgA), or a combination thereof. In some embodiments, the other agent comprises at least a portion of an IgG. In some embodiments, the IgG is IgG4. In some embodiments, the antibody is an anti-PD-1 antibody. In some embodiments, the antibody binds to or targets a ligand of PD-1. In some embodiments, the antibody is an anti-PD-L1 antibody. In some embodiments, the antibody is an anti-PD-L2 antibody. In some embodiments, the antibody is from a mammal, avian, reptile, or amphibian. In some embodiments, the antibody is from a mammal. Examples of mammals include, but are not limited to, humans, apes, monkeys, dogs, cats, rabbits, goat, sheep, cows, pigs, mice, and rats. In some embodiments, the mammal is a human. In some embodiments, the mammal is a non-human primate.

In some embodiments, the other agent comprises a fusion protein. In some embodiments, the fusion protein comprises at least a portion of an antibody. In some embodiments, the fusion protein comprises at least a portion of a non-antibody protein. In some embodiments, the fusion protein comprises at least a portion of an antibody and at least a portion of a non-antibody protein. In some embodiments, the antibody targets a component of the PD-1 pathway. For example, in some embodiments, the antibody targets PD-1. In some embodiments, the antibody targets a ligand of PD-1 (e.g., PD-L1, PD-L2). In some embodiments, the non-antibody protein comprises a component of the PD-1 pathway. For example, in some embodiments, the non-antibody protein comprises at least a portion of a ligand of PD-1. In some embodiments, the non-antibody portion comprises at least a portion of PD-L1. In some embodiments, the non-antibody portion comprises at least a portion of PD-L2. In some embodiments, the non-antibody portion comprises at least a portion of PD-1. In some embodiments, the other agent is AMP-224.

In some embodiments, the other agent that antagonizes a PD-1 pathway is an antagonist of a component of the PD-1 pathway. In some embodiments, the other agent antagonizes PD-1, a ligand of PD-1 (such as PD-L1 or PD-L2), or a combination thereof. In some embodiments, the other agent is selected from the group consisting of AMP-224, nivolumab, AMP-514, pembrolizumab, pidilizumab, REGN2810, PDR001, BGB-A317, BMS-936559, MEDI4736, MSB0010718C and MPDL3280A. In some embodiments, the other agent is nivolumab. In some embodiments, the other agent is pembrolizumab. In some embodiments, the other agent is pidilizumab. In some embodiments, the other agent is AMP-514. In some embodiments, the other agent is AMP-224.

In some embodiments, the other agent that antagonizes a PD-1 pathway in a cell is a PD-1 antagonist. In some embodiments, the antagonist of PD-1 disrupts or interferes with the interaction between PD-1 and a ligand of PD-1. In some embodiments, the antagonist of PD-1 disrupts or interferes with the interaction between PD-1 and PD-L1. In some embodiments, the antagonist of PD-1 disrupts or interferes with the interaction between PD-1 and PD-L2. In some embodiments, the PD-1 antagonist comprises a polypeptide. In some embodiments, a polypeptide comprises one or more amino acids. In some embodiments, the PD-1 antagonist comprises a fusion protein. In some embodiments, the fusion protein is AMP-224. In some embodiments, the PD-1 antagonist comprises an antibody or fragment thereof. In some embodiments, the PD-1 antagonist comprises a fully human monoclonal antibody or fragment thereof. In some embodiments, the PD-1 antagonist comprises an immunoglobulin or fragment thereof. In some embodiments, the PD-1 antagonist comprises at least a portion of an immunoglobulin G (IgG). In some embodiments, the PD-1 antagonists comprises a fusion peptide. In some embodiments, the PD-1 antagonist comprises an anti-PD-1 antibody. In some embodiments, the PD-1 antagonist comprises a monoclonal anti-PD-1 antibody. In some embodiments, the PD-1 antagonist comprises a human anti-PD-1 antibody. In some embodiments, the PD-1 antagonist comprises a humanized anti-PD-1 antibody. In some embodiments, the PD-1 antagonist comprises a fully human anti-PD-1 antibody. In some embodiments, the PD-1 antagonist comprises a chimeric anti-PD-1 antibody. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the PD-1 antagonist is nivolumab.

In some embodiments, the other agent that antagonizes a PD-1 pathway in a cell is a PD-L1 antagonist. In some embodiments, the antagonist of PD-L1 disrupts or interferes with the interaction between PD-L1 and another component of the PD-1 pathway. In some embodiments, the antagonist of PD-L1 disrupts or interferes with the interaction between PD-1 and PD-L1. In some embodiments, the PD-L1 antagonist comprises a polypeptide. In some embodiments, a polypeptide comprises one or more amino acids. In some embodiments, the PD-L1 antagonist comprises a fusion protein. In some embodiments, the fusion protein is AMP-224. In some embodiments, the PD-L1 antagonist comprises an antibody or fragment thereof. In some embodiments, the PD-L1 antagonist comprises a fully human monoclonal antibody or fragment thereof. In some embodiments, the PD-L1 antagonist comprises an immunoglobulin or fragment thereof. In some embodiments, the PD-L1 antagonist comprises at least a portion of an immunoglobulin G (IgG). In some embodiments, the PD-L1 antagonists comprises a fusion peptide. In some embodiments, the PD-L1 antagonist comprises an anti-PD-L1 antibody. In some embodiments, the PD-L1 antagonist comprises a monoclonal anti-PD-L1 antibody. In some embodiments, the PD-L1 antagonist comprises a human anti-PD-L1 antibody. In some embodiments, the PD-L1 antagonist comprises a humanized anti-PD-L1 antibody. In some embodiments, the PD-L1 antagonist comprises a fully human anti-PD-L1 antibody. In some embodiments, the PD-L1 antagonist comprises a chimeric anti-PD-L1 antibody. In some embodiments, the PD-L1 antagonist is selected from the group consisting of BMS-936559, MEDI4736, MSB0010718C and MPDL3280A (RG7446)). In some embodiments, a PD-L1 antagonist is BMS-936559. In some embodiments, a PD-L1 antagonist is MEDI4736. In some embodiments, a PD-L1 antagonist is MPDL3280A. In some embodiments, the PD-L1 antagonist is MSB0010718C.

In some embodiments, the other agent that antagonizes a PD-1 pathway in a cell is a PD-L2 antagonist. In some embodiments, the antagonist of PD-L2 disrupts or interferes with the interaction between PD-L2 and another component of the PD-1 pathway. In some embodiments, the antagonist of PD-L2 disrupts or interferes with the interaction between PD-1 and PD-L2. In some embodiments, the PD-L2 antagonist comprises a polypeptide. In some embodiments, a polypeptide comprises one or more amino acids. In some embodiments, the PD-L2 antagonist comprises a fusion protein. In some embodiments, the fusion protein is AMP-224. In some embodiments, the PD-L2 antagonist comprises an antibody or fragment thereof. In some embodiments, the PD-L2 antagonist comprises a fully human monoclonal antibody or fragment thereof. In some embodiments, the PD-L2 antagonist comprises an immunoglobulin or fragment thereof. In some embodiments, the PD-L2 antagonist comprises at least a portion of an immunoglobulin G (IgG). In some embodiments, the PD-L2 antagonists comprises a fusion peptide. In some embodiments, the PD-L2 antagonist comprises an anti-PD-L2 antibody. In some embodiments, the PD-L2 antagonist comprises a monoclonal anti-PD-L2 antibody. In some embodiments, the PD-L2 antagonist comprises a human anti-PD-L2 antibody. In some embodiments, the PD-L2 antagonist comprises a humanized anti-PD-L2 antibody. In some embodiments, the PD-L2 antagonist comprises a fully human anti-PD-L2 antibody. In some embodiments, the PD-L2 antagonist comprises a chimeric anti-PD-L2 antibody.

In some embodiments, the other agent is AMP-224. AMP-224 is a recombinant fusion protein comprising an extracellular domain of the PD-1 ligand programmed cell death ligand 2 (PD-L2) and an Fc region of human IgG. Many cancers and chronic infectious diseases can actively evade and suppress the immune system. On a molecular level, this evasion may be due in part to the interactions between the proteins PD-1 and B7-H1. AMP-224 can block the interaction between PD-1 and B7-H1. AMP-224 can overcome immune suppression, thereby allowing the immune system to fight successfully against cancer. Synonyms for AMP-224 include, but are not limited to, B7-DC Fc. AMP-224 is described, for example, in Infante et al., J Clin Oncol, 31(suppl 15):abstr 3044 (2013), Kim and Eder, Oncology, 28 Suppl 3:15-28 (2014), Kojima et al., J Immunother, 37(3):147-54 (2014), Freeman-Keller and Weber, Ther Adv Med Oncol, 7(1):12-21 (2015), and Niezgoda et al., Biomed Res Int, 2015:851387 (2015), each of which is incorporated by reference in its entirety.

In some embodiments, the other agent is BMS-936559. BMS-936559 is a fully human IgG4 anti-PD-L1 mAb that inhibits the binding of the PD-L1 ligand to both PD-1 and CD80. BMS-936559 targets one of the immunosuppressive ligands for PD-1, PD-L1, which is often expressed on tumor, stromal and immune cells. Synonyms for BMS-936559 include, but are not limited to, MDX-1105. BMS-936559 is described, for example, in Brahmer et al., N Engl J Med 366(26):2455-65 (2012), Tang and Heng, Curr Oncol Rep, 25(2):98-104 (2013), and Kim and Eder, Oncology, 28 Suppl 3:15-28 (2014), Niezgoda et al., Biomed Res Int, 2015:851387 (2015), each of which is incorporated by reference in its entirety.

In some embodiments, the other agent is MEDI4736. MEDI4736 is a monoclonal antibody directed against B7H1 (B7 homolog 1; programmed cell death ligand 1) with potential immuno stimulating activity. Upon intravenous administration, MEDI4736 binds to the cell surface antigen B7H1, thereby blocking B7H1 signaling. This may activate the immune system to exert a cytotoxic T-lymphocyte (CTL) response against B7H1-expressing tumor cells. B7H1, a member of the B7 protein superfamily and a negative regulator of cytokine synthesis, is overexpressed on certain tumor cell types. Synonyms for MEDI4736 include, but are not limited to, durvalumab and anti-B7H1 monoclonal antibody. MEDI4736 is described, for example, in Kim and Eder, Oncology, 28 Suppl 3:15-28 (2014), Lutzky et al., J Clin Oncol, 32(suppl 5S):abstr 3001 (2014), Segal et al., J Clin Oncol, 32(suppl 5S):abstr 3002 (2014), Niezgoda et al., Biomed Res Int, 2015:851387 (2015), and Stewart et al., Cancer Immunol Res, 3(9):1052-62 (2015), each of which is incorporated by reference in its entirety.

In some embodiments, the other agent is MPDL3280A. MPDL3280A is a human, Fc optimized, monoclonal antibody directed against the protein ligand PD-L1 (programmed cell death-1 ligand 1) with potential immunomodulating and antineoplastic activities. MPDL3280A contains an engineered fragment crystallizable (Fc) domain designed to optimize efficacy and safety by minimizing antibody-dependent cellular cytotoxicity (ADCC). PD-L1 monoclonal antibody MPDL3280A binds to PD-L1, blocking its binding to and activation of its receptor, PD-1 (programmed death 1), which may enhance the T-cell-mediated immune response to neoplasms and reverse T-cell inactivation. In addition, by binding to PD-L1, MPDL3280A also prevents binding of this ligand to B7.1. PD-L1 is overexpressed on many human cancer cell types. PD-L1 binding to PD-1 on T-cells suppresses the immune system and results in increased immune evasion. PD-1, a transmembrane protein expressed on activated T-cells, is a negative regulator of the immune system that limits the expansion and survival of CD8+ T cells. The Fc region of MPDL3280A is modified in such a way that it does not induce either antibody-dependent cytotoxicity (ADCC) or complement-dependent cytotoxicity (CDC). Synononyms for MPDL3280A include, but are not limited to, atezolizumab and RG7466. MPDL3280A is described, for example, in Kim and Eder, Oncology, 28 Suppl 3:15-28 (2014), Cha et al., Semin Oncol, 42(3):484-7 (2015), de Guillebon et al., World J Gastrointest Oncol, 7(8):95-101 (2015), and Niezgoda et al., Biomed Res Int, 2015:851387 (2015), each of which is incorporated by reference in its entirety.

In some embodiments, the other agent is MSB0010718C. MSB0010718C is a fully human IgG1 monoclonal antibody targeting the coregulatory protein Programmed Death (PD)-Ligand 1 (PD-L1). The PD-L1/PD-1 pathway is implicated as a major mechanism by which tumors evade elimination by the immune system. The PD-L1 molecule is expressed in many cancer types, including mMCC. MSB0010718C, which blocks the interaction of PD-L1 with its receptor PD-1, may have the potential to restore effective anti-tumor T-cell responses and thereby to inhibit tumor growth. Synonyms for MSB0010718C include, but are not limited to, avelumab. MSB0010718C is described, for example, in Heery et al., J Clin Oncol, 32(suppl 5S):abstr 3064 (2014), Scarpace, Drugs Context, 4:212289 (2015), Garon, Semin Oncol, 42 Suppl 2:S11-8 (2015), Niezgoda et al., Biomed Res Int, 2015:851387 (2015), and Boyerinas et al., Cancer Immunol Res, 3(10):1148-57 (2015), each of which is incorporated by reference in its entirety.

In some embodiments, the other agent is nivolumab. Nivolumab is a fully human monoclonal antibody directed against the negative immunoregulatory human cell surface receptor PD-1 with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Synonyms for nivolumab include, but are not limited to, BMS-936558, MDX-1106, ONO-4538 and OPDIVO®. Nivolumab is described, for example, in Brahmer et al., J Clin Oncol, 28(19):3167-75 (2010), Topalian et al., N Engl J Med, 366(26):2443-54 (2012), Hamashi et al., J Clin Oncol, 32(suppl 5S):abstr 5511 (2014), Kim and Eder, Oncology, 28 Suppl 3:15-28 (2014), Niezgoda et al., Biomed Res Int, 2015:851387 (2015), and Tang and Heng, Curr Oncol Rep, 25(2):98-104 (2013), each of which is incorporated by reference in its entirety.

In some embodiments, the other agent is AMP-514. AMP-514 (MEDI0680) is a monoclonal antibody directed against the human programmed cell death 1 (PD-1) protein, with potential immunomodulating and antineoplastic activity. Although the exact mechanism of action is not reported, anti-PD-1 monoclonal antibody AMP-514 potentially inhibits the activation of PD-1 and its downstream signaling pathways. This may restore immune function through the activation both of T-cells and cell-mediated immune responses against PD-1 overexpressing tumor cells. PD-1, a transmembrane protein in the Ig superfamily, negatively regulates T-cell activation and effector function when activated by its ligands, and plays an important role in tumor evasion from host immunity. AMP-514 (MEDI0680) is described, for example, in Goswami et al., J Immunother Cancer, 2(Suppl 3):P73 (2014) and Infante et al., J Clin Oncol, 33(suppl 15):abstr TPS3088 (2015), each of which is incorporated by reference in its entirety.

In some embodiments, the other agent is pembrolizumab. Pembrolizumab (MK-3475) is a humanized monoclonal IgG4 antibody directed against human cell surface receptor PD-1 (programmed death-1 or programmed cell death-1) with potential immunopotentiating activity. Upon administration, pembrolizumab binds to PD-1, an inhibitory signaling receptor expressed on the surface of activated T cells, and blocks the binding to and activation of PD-1 by its ligands, which results in the activation of T-cell-mediated immune responses against tumor cells. The ligands for PD-1 include PD-L1, which is expressed on antigen presenting cells (APCs) and overexpressed on certain cancer cells, and PD-L2, which is primarily expressed on APCs. Activated PD-1 negatively regulates T-cell activation through the suppression of the PI3K/Akt pathway. Synonyms for pembrolizumab include, but are not limited to, KEYTRUDA®, lambrolizumab, and MK-3475. Pembrolizumab is described, for example, in Hamid et al., N Engl J Med, 369(2):134-44 (2013), Tang and Heng, Curr Oncol Rep, 25(2):98-104 (2013), Kim and Eder, Oncology, 28 Suppl 3:15-28 (2014), Niezgoda et al., Biomed Res Int, 2015:851387 (2015), and Tsai et al., Hum Vaccin Immunother, 10(11):3111-6 (2014), each of which is incorporated by reference in its entirety.

In some embodiments, the other agent is pidilizumab. Pidilizumab (CT-011) is a humanized monoclonal antibody directed against human PD-1 (programmed cell death 1; PDCD1), with immunomodulating and antitumor activities. Pidilizumab blocks interaction between the receptor PD-1 with its ligands, PD-1 ligand 1 (PD-1L1) and PD-1 ligand 2 (PD-1L2), resulting in the attenuation of apoptotic processes in lymphocytes, primarily effector/memory T cells, and the augmentation of the anti-tumor activities of NK cells. PD-1 is an inhibitory receptor belonging to the B7-receptor family that is expressed on lymphocytes and myeloid cells; its ligands, PD-1L1 and PD-1L2, are expressed not only by hematopoietic cells but also by cells in non-lymphoid tissues. Synonyms for pidilizumab include, but are not limited to, BAT mAb and CT-011. Pidilizumab is described, for example, in Jacobsen, J Clin Oncol, 31(33):4268-70 (2013), Armand et al., J Clin Oncol, 31(33):4199-206 (2013), Atkins et al., J Clin Oncol, 32(suppl 5S):abstr 9001 (2014), Kim and Eder, Oncology, 28 Suppl 3:15-28 (2014), and Pal et al., Clin Adv Hematol Oncol, 12(2):90-9 (2014), each of which is incorporated by reference in its entirety.

In some embodiments, the other agent is REGN2810. REGN2810 is a fully human directed to the PD-1 receptor that blocks the interaction of PD-1 with its ligands, PD-L-1 and PD-L2. REGN2810 may enhance the immune response to tumor antigens. REGN2810 is described, for example, in Burova et al., Cancer Research, 75(15 Suppl):abstr 266 (2015), which is incorporated by reference in its entirety.

In some embodiments, the other agent is PDR001. PDR001 is a fully humanized monoclonal antibody directed against directed against PD-1. PDR001 may have immune checkpoint inhibitory and anti-neoplastic activities. PDR001 binds to PD-1 expressed on activated T cells and blocks the interaction with its ligands, PD-L1 and PD-L2. The inhibition of ligand binding prevents PD-1-mediated signaling and results in both T-cell activation and induction of T-cell mediated immune responses against tumor cells. PDR001 is described, for example, in National Cancer Institute Drug Dictionary and in clinical trial numbers NCT02608268, NCT02605967, NCT02460024, and NCT0240441, each of which is incorporated by reference in its entirety.

In some embodiments, the other agent is BGB-A317. BGB-A317 is a monoclonal antibody directed against PD-1. BGB-A317 may have immune checkpoint inhibitory and anti-neoplastic activities. BGB-A317 binds to PD-1 and inhibits the binding of PD-1 to its ligands, PD-L1 and PD-L2. The inhibition of ligand binding may prevent activation of PD-1 and its downstream signaling pathways. Prevention of PD-1 activation may restore immune function through the activation of both T-cells and T-cell-mediated immune responses against tumor cells. BGB-A317 is described, for example, in National Cancer Institute Drug Dictionary and in clinical trial number NCT02407990, each of which is incorporated by reference in its entirety.

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®); and b) an effective amount of a PD-1 pathway antagonist. In some embodiments, the proliferative disease is a cancer described in Section V-B, infra. In some embodiments, the proliferative disease is a cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV non-small cell lung cancer (NSCLC). In some embodiments, the cancer is pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the other agent is a PD-1 pathway antagonist. In some embodiments, the PD-1 pathway antagonist is administered via intravenous administration. In some embodiments, the PD-1 pathway antagonist is administered via intra-arterial administration. In some embodiments, the PD-1 pathway antagonist is administered via intraperitoneal administration. In some embodiments, the PD-1 pathway antagonist is administered via intrapulmonary administration. In some embodiments, the PD-1 pathway antagonist is administered via oral administration. In some embodiments, the PD-1 pathway antagonist is administered via inhalation. In some embodiments, the PD-1 pathway antagonist is administered via intravesicular administration. In some embodiments, the PD-1 pathway antagonist is administered via intramuscular administration. In some embodiments, the PD-1 pathway antagonist is administered via intra-tracheal administration. In some embodiments, the PD-1 pathway antagonist is administered via subcutaneous administration. In some embodiments, the PD-1 pathway antagonist is administered via intraocular administration. In some embodiments, the PD-1 pathway antagonist is administered via intrathecal administration. In some embodiments, the PD-1 pathway antagonist is administered via transmucosal administration. In some embodiments, the PD-1 pathway antagonist is administered via transdermal administration. In some embodiments, the PD-1 pathway antagonist is administered as a sustained continuous release formulation. In some embodiments, the PD-1 pathway antagonist is administered via an inhaler or other air borne delivery system. Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, the nivolumab is administered to the individual in a mode of administration described in Section V-C, infra. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin); and b) an effective amount of a PD-1 pathway antagonist, wherein the composition is administered intravenously, and wherein the PD-1 pathway antagonist is administered orally. In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®); and b) an effective amount of a PD-1 pathway antagonist, wherein the composition is administered intravenously and wherein the PD-1 pathway antagonist is administered orally. In some embodiments, the composition and the PD-1 pathway antagonist are administered concurrently. In some embodiments, the proliferative disease is cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the breast cancer is a triple negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is a pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is between about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, the nivolumab is administered to the individual in a dosing schedule as described in Section V-C, infra. In some embodiments, the nivolumab is administered to the individual at a dose or dose range described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin), wherein the taxane (such as paclitaxel) is in the dosage range of about 60-300 mg/m² (including for example about 80-200 mg/m², for example about 100 mg/m²), and b) a PD-1 pathway antagonist in the dosage range of about 0.01-20 mg/kg (including for example about 0.1-10 mg/kg, such as 3 mg/kg). In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 60-300 mg/m² (including for example about 80-200 mg/m², for example about 100 mg/m²), and b) about 50-1000 mg/day (including for example about 200-500, such as 400 mg/day) a PD-1 pathway antagonist in the dosage range of about 0.01-20 mg/kg (including for example about 0.1-10 mg/kg, such as 3 mg/kg). In some embodiments, the composition is administered intravenously. In some embodiments, the PD-1 pathway antagonist is administered orally. In some embodiments, the proliferative disease is cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is a pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is about 60 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is about 60 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the composition is administered as a sustained continuous release formulation. In some embodiments, the composition is administered via an inhaler or other air borne delivery system. In some embodiments, the PD-1 pathway antagonist is administered via intravenous administration. In some embodiments, the PD-1 pathway antagonist is administered via intra-arterial administration. In some embodiments, the PD-1 pathway antagonist is administered via intraperitoneal administration. In some embodiments, the PD-1 pathway antagonist is administered via intrapulmonary administration. In some embodiments, the PD-1 pathway antagonist is administered via oral administration. In some embodiments, the PD-1 pathway antagonist is administered via inhalation. In some embodiments, the PD-1 pathway antagonist is administered via intravesicular administration. In some embodiments, the PD-1 pathway antagonist is administered via intramuscular administration. In some embodiments, the PD-1 pathway antagonist is administered via intra-tracheal administration. In some embodiments, the PD-1 pathway antagonist is administered via subcutaneous administration. In some embodiments, the PD-1 pathway antagonist is administered via intraocular administration. In some embodiments, the PD-1 pathway antagonist is administered via intrathecal administration. In some embodiments, the PD-1 pathway antagonist is administered via transmucosal administration. In some embodiments, the PD-1 pathway antagonist is administered via transdermal administration. In some embodiments, the PD-1 pathway antagonist is administered as a sustained continuous release formulation. In some embodiments, the PD-1 pathway antagonist is administered via an inhaler or other air borne delivery system. Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, nivolumab is administered to the individual in a dosing schedule as described in Section V-C, infra. In some embodiments, nivolumab is administered to the individual at a dose or dose range described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating breast cancer (such as HER2 negative breast cancer) in an individual, comprising administering to the individual: a) an effective amount of a composition comprising nanoparticles comprising a taxane (such as paclitaxel) and a carrier protein (such as albumin); and b) an effective amount of a PD-1 pathway antagonist. In some embodiments, there is provided a method of treating breast cancer (such as HER2 negative breast cancer) in an individual, comprising administering to the individual: a) an effective amount of a composition comprising nanoparticles comprising paclitaxel and albumin (such as nab-paclitaxel, e.g., ABRAXANE®) and b) an effective amount of a PD-1 pathway antagonist. In some embodiments, there is provided a method of treating breast cancer (such as HER2 negative breast cancer) in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 pathway antagonist. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, nivolumab is administered to the individual in a dosing schedule as described in Section V-C, infra. In some embodiments, nivolumab is administered to the individual at a dose or dose range described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating pancreatic cancer in an individual, comprising administering to the individual: a) an effective amount of a composition comprising nanoparticles comprising a taxane (such as paclitaxel) and a carrier protein (such as albumin); and b) an effective amount of a PD-1 pathway antagonist. In some embodiments, there is provided a method of treating pancreatic cancer in an individual, comprising administering to the individual: a) an effective amount of a composition comprising nanoparticles comprising paclitaxel and albumin (such as nab-paclitaxel, e.g., ABRAXANE®); and b) an effective amount of a PD-1 pathway antagonist. In some embodiments, there is provided a method of treating pancreatic cancer in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 pathway antagonist. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, nivolumab is administered to the individual in a dosing schedule as described in Section V-C, infra. In some embodiments, nivolumab is administered to the individual at a dose or dose range described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, there is provided a method of treating lung cancer (such as NSCLC) in an individual, comprising administering to the individual: a) an effective amount of a composition comprising nanoparticles comprising a taxane (such as paclitaxel) and a carrier protein (such as albumin); and b) an effective amount of a PD-1 pathway antagonist. In some embodiments, there is provided a method of treating lung cancer (such as NSCLC) in an individual, comprising administering to the individual: a) an effective amount of a composition comprising nanoparticles comprising paclitaxel and albumin (such as nab-paclitaxel, e.g., ABRAXANE®); and b) an effective amount of a PD-1 pathway antagonist. In some embodiments, there is provided a method of treating lung cancer (such as NSCLC) in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 pathway antagonist. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². Suitable agents that antagonize a PD-1 pathway in a cell include, but are not limited to, AMP-224, nivolumab (BMS-936558), AMP-514, pembrolizumab (MK-3475), pidilizumab (CT-011), BMS-936559, MEDI4736, MSB0010718C, MPDL3280A (RG7446) and rHIgM12B7). In some embodiments, the PD-1 pathway antagonist is a PD-1 antagonist. In some embodiments, the PD-1 pathway antagonist is an antagonist of a PD-1 ligand. In some embodiments, the PD-1 pathway antagonist is a PD-L1 antagonist. In some embodiments, the PD-1 pathway antagonist is PD-L2 antagonist. In some embodiments, the PD-1 pathway antagonist is nivolumab. In some embodiments, nivolumab is administered to the individual in a dosing schedule as described in Section V-C, infra. In some embodiments, nivolumab is administered to the individual at a dose or dose range described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 pathway antagonist is pembrolizumab. In some embodiments, pembrolizumab is administered to the individual in a dosing schedule as described in Section V-C, infra. In some embodiments, pembrolizumab is administered to the individual at a dose or dose range described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 pathway antagonist is pidilizumab. In some embodiments, pidilizumab is administered to the individual in a dosing schedule as described in Section V-C, infra. In some embodiments, pidilizumab is administered to the individual at a dose or dose range described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle. In some embodiments, the PD-1 pathway antagonist is AMP-514. In some embodiments, the PD-1 pathway antagonist is AMP-514. In some embodiments, AMP-514 is administered to the individual in a dosing schedule as described in Section V-C, infra. In some embodiments, AMP-514 is administered to the individual at a dose or dose range described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 pathway antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

The methods described herein are suitable for treating various cancers, such as cancers described herein, including a cancer selected from the group consisting of lung cancer, pancreatic cancer, and breast cancer.

In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of at least one other agent that antagonizes a ligand of PD-1. In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of an antagonist of PD-L1. Suitable agents that antagonize a ligand of PD-1 include, but are not limited to, BMS-936559, MEDI4736, MSB0010718C and MPDL3280A (RG7446). In some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of an antagonist of PD-L2. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the antagonist of PD-L2 is an anti-PD-L2 antibody. In some embodiments, the proliferative disease is cancer. In some embodiments, the cancer is selected from the group consisting lung cancer, pancreatic cancer, and breast cancer. In some embodiments, the cancer is lung cancer. In some embodiments, the lung cancer is NSCLC. In some embodiments, the cancer is breast cancer. In some embodiments, the breast cancer is HER2 negative breast cancer. In some embodiments, the cancer is pancreatic cancer. In some embodiments, the PD-L1 antagonist is BMS-936559. In some embodiments, BMS-936559 is administered to the individual in a dosing schedule as described in Section V-C, infra. In some embodiments, BMS-936559 is administered to the individual at a dose or dose range described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-L1 antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-L1 antagonist is MSB0010718C. In some embodiments, MSB0010718C is administered to the individual in a dosing schedule as described in Section V-C, infra. In some embodiments, MSB0010718C is administered to the individual at a dose or dose range described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-L1 antagonist is MPDL3280A. In some embodiments, MPDL3280A is administered to the individual in a dosing schedule as described in Section V-C, infra. In some embodiments, MPDL3280A is administered to the individual at a dose or dose range described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-L1 antagonist is AMP-224. In some embodiments, AMP-224 is administered to the individual in a dosing schedule as described in Section V-C, infra. In some embodiments, AMP-224 is administered to the individual at a dose or dose range described in Section V-C, infra. In some embodiments, AMP-224 is administered to the individual in a mode of administration described in Section V-C, infra. In some embodiments, between about 0.01 mg/kg to about 10 mg/kg of AMP-224 is administered. In some embodiments, between about 0.5 mg/kg to about 10 mg/kg of AMP-224 is administered. In some embodiments, between about 0.1 mg/kg to about 10 mg/kg of AMP-224 is administered. In some embodiments, between about 1 mg/kg to about 10 mg/kg of AMP-224 is administered. In some embodiments, between about 3 mg/kg to about 10 mg/kg of AMP-224 is administered. In some embodiments, about 10 mg/kg of AMP-224 is administered. In some embodiments, at least about 7 mg/kg of AMP-224 is administered. In some embodiments, at least about 5 mg/kg of AMP-224 is administered. In some embodiments, at least about 3 mg/kg of AMP-224 is administered. In some embodiments, at least about 1 mg/kg of AMP-224 is administered. In some embodiments, at least about 0.3 mg/kg of AMP-224 is administered. In some embodiments, the AMP-224 is administered at least once during a cycle. In some embodiments, the AMP-224 is administered at least twice during a cycle. In some embodiments, the AMP-224 is administered at least once a week. In some embodiments, the AMP-224 is administered at least once every two weeks. In some embodiments, the AMP-224 is administered at least once every three weeks. In some embodiments, the AMP-224 is administered at least once every four weeks. In some embodiments, a cycle is 21 days. In some embodiments, a cycle is 28 days. In some embodiments, the AMP-224 is administered on days 1 and 15 of a 28 day cycle. In some embodiments, the AMP-224 is administered on day 15 of a 21 day cycle. In some embodiments, the AMP-224 is administered on days 1 and 15 of a 21 day cycle.

In some embodiments, the other agent is a PD-1 antagonist. Thus, for example, in some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin); and b) an effective amount of a PD-1 antagonist. In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®); and b) an effective amount of a PD-1 antagonist. In some embodiments, the proliferative disease is cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is a pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the PD-1 antagonist is administered via intravenous administration. In some embodiments, the PD-1 antagonist is administered via intra-arterial administration. In some embodiments, the PD-1 antagonist is administered via intraperitoneal administration. In some embodiments, the PD-1 antagonist is administered via intrapulmonary administration. In some embodiments, the PD-1 antagonist is administered via oral administration. In some embodiments, the PD-1 antagonist is administered via inhalation. In some embodiments, the PD-1 antagonist is administered via intravesicular administration. In some embodiments, the PD-1 antagonist is administered via intramuscular administration. In some embodiments, the PD-1 antagonist is administered via intra-tracheal administration. In some embodiments, the PD-1 antagonist is administered via subcutaneous administration. In some embodiments, the PD-1 antagonist is administered via intraocular administration. In some embodiments, the PD-1 antagonist is administered via intrathecal administration. In some embodiments, the PD-1 antagonist is administered via transmucosal administration. In some embodiments, the PD-1 antagonist is administered via transdermal administration. In some embodiments, the effective amount of the PD-1 antagonist is administered as a sustained continuous release formulation. In some embodiments, the PD-1 antagonist is administered via an inhaler or other air borne delivery system. In some embodiments, the PD-1 antagonist is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 antagonist is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 antagonist is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 antagonist is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra.

In some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin); and b) an effective amount of a PD-1 antagonist, wherein the nanoparticle composition is administered intravenously, and wherein the PD-1 antagonist is administered orally. In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®); and b) an effective amount of anti-PD-1 antibody, wherein the nanoparticle composition is administered intravenously and wherein the PD-1 antagonist is administered orally. In some embodiments, the nanoparticle composition and the PD-1 antagonist are administered concurrently. In some embodiments, the proliferative disease is cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a triple negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is a pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the PD-1 antagonist is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 antagonist is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 antagonist is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 antagonist is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra.

In some embodiments, there is provided a method of treating a proliferative disease (such as cancer) in an individual, comprising administering to the individual: a) an effective amount of a composition comprising nanoparticles comprising a taxane (such as paclitaxel) and a carrier protein (such as albumin), wherein the taxane (such as paclitaxel) is in the dosage range of about 60-300 mg/m² (including for example about 80-200 mg/m², for example about 100 mg/m²), and b) a PD-1 antagonist in the dosage range of about 0.01-20 mg/kg (including for example about 0.1-10 mg/kg, such as 3 mg/kg). In some embodiments, there is provided a method of treating a proliferative disease (such as cancer) in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 60-300 mg/m² (including for example about 80-200 mg/m², for example about 100 mg/m²), and b) about 50-1000 mg/day (including for example about 200-500, such as 400 mg/day) of a PD-1 antagonist in the dosage range of about 0.01-20 mg/kg (including for example about 0.1-10 mg/kg, such as 3 mg/kg). In some embodiments, the nanoparticle composition is administered intravenously. In some embodiments, the PD-1 antagonist is administered orally. In some embodiments, the proliferative disease is cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is a pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is about 60 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is about 60 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the PD-1 antagonist is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 antagonist is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 antagonist is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 antagonist is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra.

In some embodiments, there is provided a method of treating breast cancer (such as HER2 negative breast cancer) in an individual, comprising administering to the individual: a) an effective amount of a composition comprising nanoparticles comprising a taxane (such as paclitaxel) and a carrier protein (such as albumin); and b) an effective amount of a PD-1 antagonist. In some embodiments, there is provided a method of treating breast cancer (such as HER2 negative breast cancer) in an individual, comprising administering to the individual: a) an effective amount of a composition comprising nanoparticles comprising paclitaxel and albumin (such as nab-paclitaxel, e.g., ABRAXANE®); and b) an effective amount of a PD-1 antagonist. In some embodiments, there is provided a method of treating breast cancer (such as HER2 negative breast cancer) in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 antagonist. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the PD-1 antagonist is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 antagonist is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 antagonist is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 antagonist is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the PD-1 antagonist is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the PD-1 antagonist is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the PD-1 antagonist is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In some embodiments, there is provided a method of treating pancreatic cancer in an individual, comprising administering to the individual: a) an effective amount of a composition comprising nanoparticles comprising a taxane (such as paclitaxel) and a carrier protein (such as albumin); and b) an effective amount of a PD-1 antagonist. In some embodiments, there is provided a method of treating pancreatic cancer in an individual, comprising administering to the individual: a) an effective amount of a composition comprising nanoparticles comprising paclitaxel and albumin (such as nab-paclitaxel, e.g., ABRAXANE®); and b) an effective amount of a PD-1 antagonist. In some embodiments, there is provided a method of treating pancreatic cancer in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 antagonist. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the PD-1 antagonist is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the PD-1 antagonist is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the PD-1 antagonist is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the PD-1 antagonist is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the PD-1 antagonist is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the PD-1 antagonist is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the PD-1 antagonist is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In some embodiments, there is provided a method of treating lung cancer (such as NSCLC) in an individual, comprising administering to the individual: a) an effective amount of a composition comprising nanoparticles comprising a taxane (such as paclitaxel) and a carrier protein (such as albumin); and b) an effective amount of a PD-1 antagonist. In some embodiments, there is provided a method of treating lung cancer (such as NSCLC) in an individual, comprising administering to the individual: a) an effective amount of a composition comprising nanoparticles comprising paclitaxel and albumin (such as nab-paclitaxel, e.g., ABRAXANE®); and b) an effective amount of a PD-1 antagonist. In some embodiments, there is provided a method of treating lung cancer (such as NSCLC) in an individual, comprising: a) intravenously administering an effective amount of a composition comprising nanoparticles comprising paclitaxel and albumin (such as nab-paclitaxel, e.g., ABRAXANE®); b) intravenously or subcutaneously administering an effective amount of a PD-1 antagonist. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the PD-1 antagonist is nivolumab. In some embodiments, the PD-1 antagonist is AMP-514. In some embodiments, the PD-1 antagonist is pembrolizumab (MK-3475). In some embodiments, the PD-1 antagonist is pidilizumab (CT-011). In some embodiments, the PD-1 antagonist is REGN2810. In some embodiments, the PD-1 antagonist is PDR001. In some embodiments, the PD-1 antagonist is BGB-A317.

The methods described herein are suitable for treating various cancers, including a cancer selected from the group consisting of lung cancer, pancreatic cancer, and breast cancer.

In some embodiments, there is provided a method of treating lung cancer in an individual comprising administering to the individual: a) an effective amount of a composition comprising nanoparticles comprising a taxane (such as paclitaxel) and a carrier protein (such as albumin), and b) an effective amount of a PD-1 pathway antagonist. In some embodiments, there is provided a method of treating lung cancer in an individual comprising administering to the individual: a) an effective amount of a composition comprising nanoparticles comprising a taxane (such as paclitaxel) and a carrier protein (such as albumin), and b) an effective amount of an anti-PD-1 antibody.

In some embodiments, there is provided a method of treating lung cancer in an individual comprising administering to the individual: a) an effective amount of a composition comprising nanoparticles comprising paclitaxel coated with an albumin (such as nab-paclitaxel, e.g., ABRAXANE®), and b) an effective amount of a PD-1 pathway antagonist. In some embodiments, there is provided a method of treating lung cancer in an individual comprising administering to the individual: a) an effective amount of a composition comprising nanoparticles comprising paclitaxel coated with an albumin (such as nab-paclitaxel, e.g., ABRAXANE®), and b) an effective amount of an anti-PD-1 antibody. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered intravenously. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the anti-PD-1 antibody is administered intravenously or intraperitoneally. In some embodiments, the anti-PD-1 antibody is administered via intra-arterial administration. In some embodiments, the anti-PD-1 antibody is administered via intrapulmonary administration. In some embodiments, the anti-PD-1 antibody is administered via oral administration. In some embodiments, the anti-PD-1 antibody is administered via inhalation. In some embodiments, the anti-PD-1 antibody is administered via intravesicular administration. In some embodiments, the anti-PD-1 antibody is administered via intramuscular administration. In some embodiments, the anti-PD-1 antibody is administered via intra-tracheal administration. In some embodiments, the anti-PD-1 antibody is administered via subcutaneous administration. In some embodiments, the anti-PD-1 antibody is administered via intraocular administration. In some embodiments, the anti-PD-1 antibody is administered via intrathecal administration. In some embodiments, the anti-PD-1 antibody is administered via transmucosal administration. In some embodiments, the anti-PD-1 antibody is administered via transdermal administration. In some embodiments, the effective amount of the anti-PD-1 antibody is administered as a sustained continuous release formulation. In some embodiments, the anti-PD-1 antibody is administered via an inhaler or other air borne delivery system. In some embodiments, the anti-PD-1 antibody is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In some embodiments, there is provided a method of treating lung cancer in an individual, comprising administering to the individual: a) an effective amount of a composition comprising nanoparticles comprising a taxane (such as paclitaxel) and a carrier protein (such as albumin), wherein the taxane (such as paclitaxel) is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg). In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg). In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg). In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the amount of taxane (e.g., paclitaxel) in the composition is about 50 mg/m² to about 300 mg/m². In some embodiments, the amount of taxane (e.g., paclitaxel) in the composition is about 60 mg/m² to about 300 mg/m². In some embodiments, the amount of taxane (e.g., paclitaxel) in the composition is between about 75 mg/m² to about 275 mg/m². In some embodiments, the amount of taxane (e.g., paclitaxel) in the composition is between about 80 mg/m² to about 350 mg/m². In some embodiments, the amount of taxane (e.g., paclitaxel) in the composition is between about 80 mg/m² to about 300 mg/m². In some embodiments, the amount of taxane (e.g., paclitaxel) in the composition is between about 80 mg/m² to about 200 mg/m². In some embodiments, the amount of taxane (e.g., paclitaxel) in the composition is between about 80 mg/m² to about 150 mg/m². In some embodiments, the amount of taxane (e.g., paclitaxel) in the composition is between about 100 mg/m² to about 250 mg/m². In some embodiments, the amount of taxane (e.g., paclitaxel) in the composition is between about 150 mg/m² to about 350 mg/m². In some embodiments, the amount of taxane (e.g., paclitaxel) in the composition is between about 170 mg/m² to about 200 mg/m². In some embodiments, the amount of taxane (e.g., paclitaxel) in the composition is between about 200 mg/m² to about 350 mg/m². In some embodiments, the amount of taxane (e.g., paclitaxel) in the composition is about 50 mg/m². In some embodiments, the amount of taxane (e.g., paclitaxel) in the composition is about 75 mg/m². In some embodiments, the amount of taxane (e.g., paclitaxel) in the composition is about 100 mg/m². In some embodiments, the amount of taxane (e.g., paclitaxel) in the composition is about 125 mg/m². In some embodiments, the amount of taxane (e.g., paclitaxel) in the composition is about 150 mg/m². In some embodiments, the amount of taxane (e.g., paclitaxel) in the composition is about 175 mg/m². In some embodiments, the amount of taxane (e.g., paclitaxel) in the composition is about 200 mg/m². In some embodiments, the amount of taxane (e.g., paclitaxel) in the composition is about 225 mg/m². In some embodiments, the amount of taxane (e.g., paclitaxel) in the composition is about 250 mg/m². In some embodiments, the amount of taxane (e.g., paclitaxel) in the composition is about 260 mg/m². In some embodiments, the amount of taxane (e.g., paclitaxel) in the composition is about 275 mg/m². In some embodiments, the amount of taxane (e.g., paclitaxel) in the composition is about 300 mg/m². In some embodiments, the amount of taxane (e.g., paclitaxel) in the composition is about 325 mg/m². In some embodiments, the amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the nanoparticle composition is administered first followed by administration of the nivolumab. In some embodiments, the nivolumab is administered first followed by administration of the nanoparticle composition. In some embodiments, the administrations of the nanoparticle composition and the nivolumab are concurrent. In some embodiments, the nanoparticle composition is administered once a week for a three week cycle and the nivolumab is administered once during a three week cycle. In some embodiments, the nanoparticle composition is administered on days 1, 8, and 15 of a 21 day cycle and the nivolumab is administered on day 15 of a 21 day cycle. In some embodiments, the nanoparticle composition is administered three times over a four week cycle and the nivolumab is administered twice over a four week cycle. In some embodiments, the nanoparticle composition is administered on days 1, 8, and 15 of a 28 day cycle and the nivolumab is administered on days 1 and 15 of a 28 day cycle. In some embodiments, the nivolumab is administered to the individual at a dose or dose range described in Section V-C, infra. In some embodiments, the nivolumab is administered to the individual in a dosing schedule as described in Section V-C, infra. In some embodiments, the nivolumab is administered to the individual in a mode of administration described in Section V-C, infra. In some embodiments, the nivolumab is administered via intravenous administration. In some embodiments, the nivolumab is administered via intra-arterial administration. In some embodiments, the nivolumab is administered via intraperitoneal administration. In some embodiments, the nivolumab is administered via intrapulmonary administration. In some embodiments, the nivolumab is administered via oral administration. In some embodiments, the nivolumab is administered via inhalation. In some embodiments, the nivolumab is administered via intravesicular administration. In some embodiments, the nivolumab is administered via intramuscular administration. In some embodiments, the nivolumab is administered via intra-tracheal administration. In some embodiments, the nivolumab is administered via subcutaneous administration. In some embodiments, the nivolumab is administered via intraocular administration. In some embodiments, the nivolumab is administered via intrathecal administration. In some embodiments, the nivolumab is administered via transmucosal administration. In some embodiments, the nivolumab is administered via transdermal administration. In some embodiments, the effective amount of the nivolumab is administered as a sustained continuous release formulation. In some embodiments, the nivolumab is administered via an inhaler or other air borne delivery system.

In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg), wherein the nivolumab is administered on day 15 of a 21-day cycle and wherein the nanoparticle composition is administered on days 1, 8, and 15 of a 21-day cycle, for at least 3 cycles. In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage of about 100 mg/m², and b) nivolumab at a dosage range of about 0.01-20 mg/kg (including for example about 0.1-10 mg/kg or for example about 0.1-6 mg/kg), wherein the nivolumab is administered on day 15 of a 21-day cycle and wherein the nanoparticle composition is administered on days 1, 8, and 15 of a 21-day cycle, for at least 3 cycles. In some embodiments, there is provided a method of treating lung cancer in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 50-200 mg/m² (such as about 75 mg/m², 100 mg/m², 125 mg/m², or 150 mg/m²), and b) intravenously or intraperitoneally administering to the individual about 0.01-20 mg/kg (including for example about 0.1-10 mg/kg or for example about 0.1-6 mg/kg) of nivolumab, wherein the nivolumab is administered on day 15 of a 21-day cycle and wherein the nanoparticle composition is administered on days 1, 8, and 15 of a 21-day cycle, for at least 3 cycles. In some embodiments, there is provided a method of treating lung cancer in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 100-150 mg/m² (such as 100 or 125 mg/m²) and b) intravenously or intraperitoneally administering to the individual about 0.01-20 mg/kg (including for example about 0.1-10 mg/kg or for example about 0.1-6 mg/kg) of nivolumab, wherein the nivolumab is administered on day 15 of a 21-day cycle and wherein the nanoparticle composition is administered on days 1, 8, and 15 of a 21-day cycle, for at least 3 cycles. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the amount of taxane (e.g., paclitaxel) in the composition is about 50 mg/m² to about 300 mg/m². In some embodiments, the amount of taxane (e.g., paclitaxel) in the composition is about 60 mg/m² to about 300 mg/m². In some embodiments, the amount of taxane (e.g., paclitaxel) in the composition is between about 75 mg/m² to about 275 mg/m². In some embodiments, the amount of taxane (e.g., paclitaxel) in the composition is between about 80 mg/m² to about 350 mg/m². In some embodiments, the amount of taxane (e.g., paclitaxel) in the composition is between about 80 mg/m² to about 300 mg/m². In some embodiments, the amount of taxane (e.g., paclitaxel) in the composition is between about 80 mg/m² to about 200 mg/m². In some embodiments, the amount of taxane (e.g., paclitaxel) in the composition is between about 80 mg/m² to about 150 mg/m². In some embodiments, the amount of taxane (e.g., paclitaxel) in the composition is between about 100 mg/m² to about 250 mg/m². In some embodiments, the amount of taxane (e.g., paclitaxel) in the composition is between about 150 mg/m² to about 350 mg/m². In some embodiments, the amount of taxane (e.g., paclitaxel) in the composition is between about 170 mg/m² to about 200 mg/m². In some embodiments, the amount of taxane (e.g., paclitaxel) in the composition is between about 200 mg/m² to about 350 mg/m². In some embodiments, the amount of taxane (e.g., paclitaxel) in the composition is about 50 mg/m². In some embodiments, the amount of taxane (e.g., paclitaxel) in the composition is about 75 mg/m². In some embodiments, the amount of taxane (e.g., paclitaxel) in the composition is about 100 mg/m². In some embodiments, the amount of taxane (e.g., paclitaxel) in the composition is about 125 mg/m². In some embodiments, the amount of taxane (e.g., paclitaxel) in the composition is about 150 mg/m². In some embodiments, the amount of taxane (e.g., paclitaxel) in the composition is about 175 mg/m². In some embodiments, the amount of taxane (e.g., paclitaxel) in the composition is about 200 mg/m². In some embodiments, the amount of taxane (e.g., paclitaxel) in the composition is about 225 mg/m². In some embodiments, the amount of taxane (e.g., paclitaxel) in the composition is about 250 mg/m². In some embodiments, the amount of taxane (e.g., paclitaxel) in the composition is about 260 mg/m². In some embodiments, the amount of taxane (e.g., paclitaxel) in the composition is about 275 mg/m². In some embodiments, the amount of taxane (e.g., paclitaxel) in the composition is about 300 mg/m². In some embodiments, the amount of taxane (e.g., paclitaxel) in the composition is about 325 mg/m². In some embodiments, the amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, nivolumab is administered starting at Cycle 1. In some embodiments, nivolumab is administered starting at Cycle 3. In some embodiments, the nanoparticle is administered during Cycles 1-4. In some embodiments, the nivolumab is administered to the individual at a dose or dose range described in Section V-C, infra. In some embodiments, the nivolumab is administered to the individual in a dosing schedule as described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the method further comprises administering one or more chemotherapeutic agents. In some embodiments, the one or more chemotherapeutic agents are administered once during a 21-day cycle. In some embodiments, the one or more chemotherapeutic agents are administered on day 1 of a 21-day cycle. In some embodiments, the one or more chemotherapeutic agents is a platinum-based therapy. In some embodiments, the platinum-based therapy is carboplatin. In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg), and c) carboplatin. In some embodiments, the carboplatin is administered at a dosage range of AUC 1-AUC 6. In some embodiments, the carboplatin is administered at a dosage of AUC 6. In some embodiments, the platinum-based therapy is cisplatin. In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg), and c) cisplatin. In some embodiments, the one or more chemotherapeutic agents is a nucleotide analog. In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg), and c) a nucleoside analog. In some embodiments, the nucleotide analog is gemcitabine. In some embodiments, there is provided a method of treating lung cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 50-350 mg/m² (including for example about 80-300 mg/m², for example about 100 mg/m², about 125 mg/m², or about 260 mg/m²), and b) nivolumab at a dosage range of about 0.01-20 mg/kg (including for example about 0.1-10 mg/kg or for example about 0.1-6 mg/kg), and c) gemcitabine. In some embodiments, the gemcitabine is administered at a dosage range of between about 500 to about 2000 mg/m². In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, nivolumab is administered as a monotherapy starting at Cycle 5. In some embodiments, the individual suffering from lung cancer has not had prior cytotoxic regimens. In some embodiments, the individual suffering from lung cancer has had no more than 2 prior cytotoxic regimens. In some embodiments, the individual suffering from lung cancer has had more than 2 prior cytotoxic regimens.

In some embodiments, the lung cancer is a non-small cell lung cancer (NSCLC). Examples of NCSLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC may be, according to TNM classifications, a stage T tumor (primary tumor), a stage N tumor (regional lymph nodes), or a stage M tumor (distant metastasis).

In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the lung cancer is small cell lung cancer (SCLC; also called oat cell carcinoma). The small cell lung cancer may be limited-stage, extensive stage or recurrent small cell lung cancer. In some embodiments, the individual may be a human who has a gene, genetic mutation, or polymorphism suspected or shown to be associated with lung cancer (e.g., SASH1, LATS1, IGF2R, PARK2, KRAS, PTEN, Kras2, Krag, Pas1, ERCC1, XPD, IL8RA, EGFR, alpha₁-AD, EPHX, MMP1, MMP2, MMP3, MMP12, IL1beta, RAS, and/or AKT) or has one or more extra copies of a gene associated with lung cancer.

In some embodiments, there is provided a method of treating pancreatic cancer in an individual comprising administering to the individual: a) an effective amount of a composition comprising nanoparticles comprising a taxane (such as paclitaxel) and a carrier protein (such as albumin), and b) an effective amount of a PD-1 pathway antagonist. In some embodiments, there is provided a method of treating pancreatic cancer in an individual comprising administering to the individual: a) an effective amount of a composition comprising nanoparticles comprising a taxane (such as paclitaxel) and a carrier protein (such as albumin), and b) an effective amount of an anti-PD-1 antibody. In some embodiments, there is provided a method of treating pancreatic cancer in an individual comprising administering to the individual: a) an effective amount of a composition comprising nanoparticles comprising paclitaxel coated with an albumin (such as nab-paclitaxel, e.g., ABRAXANE®), and b) an effective amount of a PD-1 pathway antagonist. In some embodiments, there is provided a method of treating pancreatic cancer in an individual comprising administering to the individual: a) an effective amount of a composition comprising nanoparticles comprising paclitaxel coated with an albumin (such as nab-paclitaxel, e.g., ABRAXANE®), and b) an effective amount of an anti-PD-1 antibody. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the nanoparticle has an average diameter of less than or equal to about 200 nm and the weight ratio of the carrier protein (such as albumin) to taxane (such as paclitaxel) in the composition is about 9:1. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered intravenously. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the anti-PD-1 antibody is administered intravenously or intraperitoneally. In some embodiments, the anti-PD-1 antibody is nivolumab. In some embodiments, nivolumab is administered to the individual at a dose or dose range described in Section V-C, infra. In some embodiments, nivolumab is administered to the individual in a dosing schedule as described in Section V-C, infra. In some embodiments, nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In some embodiments, there is provided a method of treating pancreatic cancer in an individual, comprising administering to the individual: a) an effective amount of a composition comprising nanoparticles comprising a taxane (such as paclitaxel) and a carrier protein (such as albumin), wherein the taxane (such as paclitaxel) is in the dosage range of about 60-300 mg/m² (including for example about 80-200 mg/m² or for example about 125 mg/m²), and b) about 0.01-20 mg/kg (including for example about 0.1-10 mg/kg or for example about 0.1-6 mg/kg) of nivolumab. In some embodiments, there is provided a method of treating pancreatic cancer in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 60-300 mg/m² (including for example about 80-200 mg/m² or for example about 125 mg/m²), and b) about 0.1 to about 15 mg/kg (including for example about 0.1 to about 7 mg/kg or for example about 5, 3, 1, or 0.3 mg/kg) of nivolumab. In some embodiments, there is provided a method of treating pancreatic cancer in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 125 mg/m², and b) about 0.01-20 mg/kg (including for example about 0.1-10 mg/kg or for example about 0.1-6 mg/kg) of nivolumab. In some embodiments, there is provided a method of treating pancreatic cancer in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 125 mg/m², and b) about 0.1 to about 15 mg/kg (including for example about 0.1 to about 7 mg/kg or for example about 5, 3, 1, or 0.3 mg/kg) of nivolumab. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the amount of taxane (e.g., paclitaxel) in the composition is between about 75 mg/m² to about 275 mg/m². In some embodiments, the amount of taxane (e.g., paclitaxel) in the composition is between about 80 mg/m² to about 350 mg/m². In some embodiments, the amount of taxane (e.g., paclitaxel) in the composition is between about 80 mg/m² to about 300 mg/m². In some embodiments, the amount of taxane (e.g., paclitaxel) in the composition is between about 80 mg/m² to about 200 mg/m². In some embodiments, the amount of taxane (e.g., paclitaxel) in the composition is between about 80 mg/m² to about 150 mg/m². In some embodiments, the amount of taxane (e.g., paclitaxel) in the composition is between about 100 mg/m² to about 250 mg/m². In some embodiments, the amount of taxane (e.g., paclitaxel) in the composition is between about 170 mg/m² to about 200 mg/m². In some embodiments, the amount of taxane (e.g., paclitaxel) in the composition is about 50 mg/m². In some embodiments, the amount of taxane (e.g., paclitaxel) in the composition is about 75 mg/m². In some embodiments, the amount of taxane (e.g., paclitaxel) in the composition is about 100 mg/m². In some embodiments, the amount of taxane (e.g., paclitaxel) in the composition is about 125 mg/m². In some embodiments, the amount of taxane (e.g., paclitaxel) in the composition is about 150 mg/m². In some embodiments, the amount of taxane (e.g., paclitaxel) in the composition is about 175 mg/m². In some embodiments, the amount of taxane (e.g., paclitaxel) in the composition is about 200 mg/m². In some embodiments, the amount of taxane (e.g., paclitaxel) in the composition is about 225 mg/m². In some embodiments, the amount of taxane (e.g., paclitaxel) in the composition is about 250 mg/m². In some embodiments, the amount of taxane (e.g., paclitaxel) in the composition is about 260 mg/m². In some embodiments, the amount of taxane (e.g., paclitaxel) in the composition is about 275 mg/m². In some embodiments, the amount of taxane (e.g., paclitaxel) in the composition is about 300 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the nivolumab is administered to the individual at a dose or dose range described in Section V-C, infra. In some embodiments, the nivolumab is administered to the individual in a dosing schedule as described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the nanoparticle composition is administered first followed by administration of the nivolumab. In some embodiments, the nivolumab is administered first followed by administration of the nanoparticle composition. In some embodiments, the administrations of the nanoparticle composition and the nivolumab are concurrent. In some embodiments, the nanoparticle composition is administered three out of four weeks and the nivolumab is administered three out of four weeks. In some embodiments, the nanoparticle composition is administered on days 1, 8, and 15 of a 28-day cycle and nivolumab is administered on days 1 and 15 of a 28-day cycle. In some embodiments, the method further comprises administering one or more chemotherapeutic agents. In some embodiments, the one or more chemotherapeutic agents comprises a nucleoside analog. In some embodiments, the nucleoside analog is gemcitabine. In some embodiments, the method further comprises administering about 500 to about 2000 mg/m² of gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In some embodiments, there is provided a method of treating pancreatic cancer in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 125 mg/m², and b) about 0.1 to about 15 mg/kg (including for example about 0.1 to about 7 mg/kg or for example about 5, 3, 1, or 0.3 mg/kg) of nivolumab, wherein the nivolumab is administered on days 1 and 15 of a 28-day cycle and the nanoparticle composition is administered on days 1, 8, and 15 of a 28-day cycle. In some embodiments, there is provided a method of treating pancreatic cancer in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 125 mg/m², and b) about 0.1 mg/kg to about 10 mg/kg (such as about 0.1 mg/kg to about 6 mg/kg) of nivolumab, wherein the nivolumab is administered on days 1 and 15 of a 28-day cycle and the nanoparticle composition is administered on days 1, 8, and 15 of a 28-day cycle. In some embodiments, there is provided a method of treating pancreatic cancer in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 100-150 mg/m² (such as 125 mg/m²) and b) intravenously or intraperitoneally administering to the individual about 0.1 mg/kg to about 10 mg/kg (such as about 0.1 mg/kg to about 6 mg/kg) of nivolumab, wherein the nivolumab is intravenously or intraperitoneally administered on days 1 and 15 of a 28-day cycle, and the nanoparticle composition is intravenously administered on days 1, 8, and 15 of a 28-day cycle. In some embodiments, the individual suffering from pancreatic cancer has not had prior cytotoxic regimens. In some embodiments, the individual suffering from pancreatic cancer has had no more than 2 prior cytotoxic regimens. In some embodiments, the individual suffering from pancreatic cancer has had more than 2 prior cytotoxic regimens. In some embodiments, the dosage of the paclitaxel is about 100 mg/m². In some embodiments, the dosage of the paclitaxel is about 125 mg/m². In some embodiments, the dosage of the paclitaxel is about 150 mg/m². In some embodiments, the nivolumab is administered to the individual at a dose or dose range described in Section V-C, infra. In some embodiments, the nivolumab is administered to the individual in a dosing schedule as described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the method further comprises administering one or more chemotherapeutic agents. In some embodiments, the one or more chemotherapeutic agents comprises a nucleoside analog. In some embodiments, the nucleoside analog is gemcitabine. In some embodiments, the method further comprises administering about 500 to about 2000 mg/m² of gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In some embodiments, the pancreatic cancer includes, but is not limited to, serous microcystic adenoma, intraductal papillary mucinous neoplasm, mucinous cystic neoplasm, solid pseudopapillary neoplasm, pancreatic adenocarcinoma, pancreatic ductal carcinoma, or pancreatoblastoma. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

In some embodiments, there is provided a method of treating breast cancer (for example, HER2 negative breast cancer or triple negative breast cancer) in an individual comprising administering to the individual: a) an effective amount of a composition comprising nanoparticles comprising a taxane (such as paclitaxel) and a carrier protein (such as albumin), and b) an effective amount of a PD-1 pathway antagonist. In some embodiments, there is provided a method of treating breast cancer (for example, HER2 negative breast cancer or triple negative breast cancer) in an individual comprising administering to the individual: a) an effective amount of a composition comprising nanoparticles comprising a taxane (such as paclitaxel) and a carrier protein (such as albumin), and b) an effective amount of an anti-PD-1 antibody. In some embodiments, there is provided a method of treating breast cancer (for example, HER2 negative breast cancer or triple negative breast cancer) in an individual comprising administering to the individual: a) an effective amount of a composition comprising nanoparticles comprising paclitaxel coated with an albumin (such as nab-paclitaxel, e.g., ABRAXANE®), and b) an effective amount of a PD-1 pathway antagonist. In some embodiments, there is provided a method of treating breast cancer (for example, HER2 negative breast cancer or triple negative breast cancer) in an individual comprising administering to the individual: a) an effective amount of a composition comprising nanoparticles comprising paclitaxel coated with an albumin (such as nab-paclitaxel, e.g., ABRAXANE®), and b) an effective amount of an anti-PD-1 antibody. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 45 mg/m² to about 350 mg/m². In some embodiments, the amount of nab-paclitaxel (e.g., ABRAXANE®) in the composition is about 50 mg/m² to about 300 mg/m². In some embodiments, the amount of nab-paclitaxel (e.g., ABRAXANE®) in the composition is about 50 mg/m² to about 350 mg/m². In some embodiments, the amount of nab-paclitaxel (e.g., ABRAXANE®) in the composition is about 60 mg/m² to about 300 mg/m². In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 350 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 150 mg/m² to about 350 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is between about 200 mg/m² to about 350 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the amount of nab-paclitaxel (e.g., ABRAXANE®) in the composition is about 325 mg/m². In some embodiments, the amount of nab-paclitaxel (e.g., ABRAXANE®) in the composition is about 350 mg/m². In some embodiments, the composition is administered intravenously. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the anti-PD-1 antibody is administered intravenously or intraperitoneally. In some embodiments, the anti-PD-1 antibody is nivolumab. In some embodiments, the nivolumab is administered to the individual at a dose or dose range described in Section V-C, infra. In some embodiments, the nivolumab is administered to the individual in a dosing schedule as described in Section V-C, infra. In some embodiments, the nivolumab is administered to the individual in a mode of administration described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle. In some embodiments, the anti-PD-1 antibody is AMP-514. In some embodiments, AMP-514 is administered to the individual at a dose or dose range described in Section V-C, infra. In some embodiments, AMP-514 is administered to the individual in a dosing schedule as described in Section V-C, infra. In some embodiments, AMP-514 is administered to the individual in a mode of administration described in Section V-C, infra. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle. In some embodiments, the anti-PD-1 antibody is pembrolizumab (MK-3475). In some embodiments, pembrolizumab is administered to the individual at a dose or dose range described in Section V-C, infra. In some embodiments, pembrolizumab is administered to the individual in a dosing schedule as described in Section V-C, infra. In some embodiments, pembrolizumab is administered to the individual in a mode of administration described in Section V-C, infra. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle. In some embodiments, the anti-PD-1 antibody is pidilizumab (CT-011). In some embodiments, pidilizumab is administered to the individual at a dose or dose range described in Section V-C, infra. In some embodiments, pidilizumab is administered to the individual in a dosing schedule as described in Section V-C, infra. In some embodiments, pidilizumab is administered to the individual in a mode of administration described in Section V-C, infra. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle. In some embodiments, the anti-PD-1 antibody is REGN2810. In some embodiments, the REGN2810 is administered to the individual in a dosing schedule as described in Section V-C, infra. In some embodiments, the REGN2810 is administered to the individual at a dose or dose range described in Section V-C, infra. In some embodiments, the REGN2810 is administered to the individual in a mode of administration described in Section V-C, infra. In some embodiments, between about 0.01 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle. In some embodiments, the anti-PD-1 antibody is PDR001. In some embodiments, the PDR001 is administered to the individual in a dosing schedule as described in Section V-C, infra. In some embodiments, the PDR001 is administered to the individual at a dose or dose range described in Section V-C, infra. In some embodiments, the PDR001 is administered to the individual in a mode of administration described in Section V-C, infra. In some embodiments, between about 0.01 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle. In some embodiments, the anti-PD-1 antibody is BGB-A317. In some embodiments, the BGB-A317 is administered to the individual in a dosing schedule as described in Section V-C, infra. In some embodiments, the BGB-A317 is administered to the individual at a dose or dose range described in Section V-C, infra. In some embodiments, the BGB-A317 is administered to the individual in a mode of administration described in Section V-C, infra. In some embodiments, between about 0.01 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In some embodiments, there is provided a method of treating breast cancer (for example, HER2 negative breast cancer or triple negative breast cancer) in an individual, comprising administering to the individual: a) an effective amount of a composition comprising nanoparticles comprising a taxane (such as paclitaxel) and a carrier protein (such as albumin), wherein the taxane (such as paclitaxel) is in the dosage range of about 60-300 mg/m² (including for example about 80-270 mg/m² or for example about 100 or 260 mg/m²), and b) about 0.1 to about 15 mg/kg (including for example about 0.1 to about 7 mg/kg or for example about 5, 3, 1, or 0.3 mg/kg) of nivolumab. In some embodiments, there is provided a method of treating breast cancer (for example, HER2 negative breast cancer or triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 60-300 mg/m² (including for example about 80-270 mg/m² or for example about 100 or 260 mg/m²), and b) about 0.1 to about 15 mg/kg (including for example about 0.1 to about 7 mg/kg or for example about 5, 3, 1, or 0.3 mg/kg) of nivolumab. In some embodiments, there is provided a method of treating breast cancer (for example, HER2 negative breast cancer or triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the paclitaxel is in the dosage range of about 100-260 mg/m², and b) about 0.1 to about 15 mg/kg (including for example about 0.1 to about 7 mg/kg or for example about 5, 3, 1, or 0.3 mg/kg) of nivolumab. In some embodiments, there is provided a method of treating breast cancer (for example, HER2 negative breast cancer or triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 100 mg/m², and b) about 0.1 to about 15 mg/kg (including for example about 0.1 to about 7 mg/kg or for example about 5, 3, 1, or 0.3 mg/kg) of nivolumab. In some embodiments, the nanoparticle composition is administered first followed by administration of the nivolumab. In some embodiments, the nivolumab is administered first followed by administration of the nanoparticle composition. In some embodiments, the administrations of the nanoparticle composition and the nivolumab are concurrent. In some embodiments, the nanoparticle composition is administered three out of four weeks and the nivolumab is administered on days 1 and 15 of a 28 day cycle. In some embodiments, the nanoparticle composition is administered on days 1, 8, and 15 of a 28-day cycle and the nivolumab is administered on days 1 and 15 of a 28-day cycle. In some embodiments, the nanoparticle composition is administered once during a 3-week cycle and the nivolumab is administered on once during a 3-week. In some embodiments, the nanoparticle composition is administered on day 1 of a 21-day cycle and the nivolumab is administered on day 15 of a 21-day cycle. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is about 60 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is about 60 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the nivolumab is administered to the individual at a dose or dose range described in Section V-C, infra. In some embodiments, the nivolumab is administered to the individual in a dosing schedule as described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle.

In some embodiments, there is provided a method of treating breast cancer (for example, HER2 negative breast cancer or for example, triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 100 mg/m², and b) about 0.1 to about 15 mg/kg (including for example about 0.1 to about 7 mg/kg or for example about 5, 3, 1, or 0.3 mg/kg) of nivolumab, wherein the nivolumab is administered on days 1 and 15 of a 28-day cycle and the nanoparticle composition is administered on days 1, 8, and 15 of a 28-day cycle. In some embodiments, there is provided a method of treating breast cancer (for example, HER2 negative breast cancer or triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 100 mg/m², and b) about 0.1 to about 15 mg/kg (including for example about 0.1 to about 7 mg/kg or for example about 5, 3, 1, or 0.3 mg/kg) of nivolumab, wherein the nivolumab is administered on days 1 and 15 of a 28-day cycle and the nanoparticle composition is administered on days 1, 8, and 15 of a 28-day cycle. In some embodiments, there is provided a method of treating breast cancer (for example, HER2 negative breast cancer or triple negative breast cancer) in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 50 to about 300 mg/m² (for example 75 mg/m², 100 mg/m², 125 mg/m², 150 mg/m², 200 mg/m², 225 mg/m², or 260 mg/m²), and b) about 0.1 to about 15 mg/kg (including for example about 0.1 to about 7 mg/kg or for example about 5, 3, 1, or 0.3 mg/kg) of nivolumab, wherein the nivolumab is intravenously or peritonneally administered on days 1 and 15 of a 28-day cycle and the nanoparticle composition is administered on days 1, 8, and 15 of a 28-day cycle. In some embodiments, there is provided a method of treating breast cancer (for example, HER2 negative breast cancer or triple negative breast cancer) in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 50-200 mg/m² (such as about 75 mg/m², 100 mg/m², 125 mg/m², or 150 mg/m²), and b) intravenously or intraperitoneally administering to the individual 0.1 to about 15 mg/kg (including for example about 0.1 to about 7 mg/kg or for example about 5, 3, 1, or 0.3 mg/kg) of nivolumab, wherein the nivolumab is intravenously or intraperitoneally administered on days 1 and 15 of a 28-day cycle and the nanoparticle composition is administered on days 1, 8, and 15 of a 28-day cycle. In some embodiments, the individual suffering from breast cancer (for example, HER2 negative breast cancer or triple negative breast cancer) has not had prior cytotoxic regimens. In some embodiments, the individual suffering from breast cancer (for example, HER2 negative breast cancer or triple negative breast cancer) has had no more than 2 prior cytotoxic regimens. In some embodiments, the individual suffering from breast cancer (for example, HER2 negative breast cancer or triple negative breast cancer) has had more than 2 prior cytotoxic regimens. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the composition comprises nab-paclitaxel, e.g., ABRAXANE®. In some embodiments, the amount of nab-paclitaxel (e.g., ABRAXANE®) in the composition is an amount described in Section V-C, infra. In some embodiments, the amount of nab-paclitaxel (e.g., ABRAXANE®) in the composition is between about 75 mg/m² to about 275 mg/m². In some embodiments, the amount of nab-paclitaxel (e.g., ABRAXANE®) in the composition is between about 80 mg/m² to about 300 mg/m². In some embodiments, the amount of nab-paclitaxel (e.g., ABRAXANE®) in the composition is about 80 mg/m² to about 200 mg/m². In some embodiments, the amount of nab-paclitaxel (e.g., ABRAXANE®) in the composition is between about 80 mg/m² to about 150 mg/m². In some embodiments, the amount of nab-paclitaxel (e.g., ABRAXANE®) in the composition is between about 100 mg/m² to about 250 mg/m². In some embodiments, the amount of nab-paclitaxel (e.g., ABRAXANE®) in the composition is between about 170 mg/m² to about 200 mg/m². In some embodiments, the amount of nab-paclitaxel (e.g., ABRAXANE®) in the composition is about 50 mg/m². In some embodiments, the amount of nab-paclitaxel (e.g., ABRAXANE®) in the composition is about 75 mg/m². In some embodiments, the amount of nab-paclitaxel (e.g., ABRAXANE®) in the composition is about 100 mg/m². In some embodiments, the amount of nab-paclitaxel (e.g., ABRAXANE®) in the composition is about 125 mg/m². In some embodiments, the amount of nab-paclitaxel (e.g., ABRAXANE®) in the composition is about 150 mg/m². In some embodiments, the amount of nab-paclitaxel (e.g., ABRAXANE®) in the composition is about 175 mg/m². In some embodiments, the amount of nab-paclitaxel (e.g., ABRAXANE®) in the composition is about 200 mg/m². In some embodiments, the amount of nab-paclitaxel (e.g., ABRAXANE®) in the composition is about 225 mg/m². In some embodiments, the amount of nab-paclitaxel (e.g., ABRAXANE®) in the composition is about 250 mg/m². In some embodiments, the amount of nab-paclitaxel (e.g., ABRAXANE®) in the composition is about 260 mg/m². In some embodiments, the amount of nab-paclitaxel (e.g., ABRAXANE®) in the composition is about 275 mg/m². In some embodiments, the amount of nab-paclitaxel (e.g., ABRAXANE®) in the composition is about 300 mg/m². In some embodiments, the nivolumab is administered to the individual at a dose or dose range described in Section V-C, infra. In some embodiments, the nivolumab is administered to the individual in a dosing schedule as described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle.

In some embodiments, there is provided a method of treating breast cancer (for example, HER2 negative breast cancer or for example, triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 260 mg/m², and b) about 0.1 to about 15 mg/kg (including for example about 0.1 to about 7 mg/kg or for example about 5, 3, 1, or 0.3 mg/kg) of nivolumab, wherein the nivolumab is administered on day 15 of a 21-day cycle and the nanoparticle composition is administered on day 1 of a 21-day cycle. In some embodiments, there is provided a method of treating breast cancer (for example, HER2 negative breast cancer or triple negative breast cancer) in an individual, comprising administering to the individual: a) an effective amount of a composition comprising nanoparticles comprising paclitaxel coated with an albumin (such as nab-paclitaxel, e.g., ABRAXANE®), wherein the dosage of the paclitaxel is about 260 mg/m², and b) about 0.1 to about 15 mg/kg (including for example about 0.1 to about 7 mg/kg or for example about 5, 3, 1, or 0.3 mg/kg) of nivolumab, wherein the nivolumab is administered on day 15 of a 21-day cycle and the nanoparticle composition is administered on day 1 of a 21-day cycle. In some embodiments, there is provided a method of treating breast cancer (for example, HER2 negative breast cancer or triple negative breast cancer) in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 50 to about 300 mg/m² (for example 75 mg/m², 100 mg/m², 125 mg/m², 150 mg/m², 200 mg/m², 225 mg/m², or 260 mg/m²), and b) about 0.1 to about 15 mg/kg (including for example about 0.1 to about 7 mg/kg or for example about 5, 3, 1, or 0.3 mg/kg) of nivolumab, wherein the nivolumab is intravenously or peritonneally administered on day 15 of a 21-day cycle and the nanoparticle composition is administered on day 1 of a 21-day cycle. In some embodiments, there is provided a method of treating breast cancer (for example, HER2 negative breast cancer or triple negative breast cancer) in an individual, comprising: a) intravenously administering to the individual an effective amount of a composition comprising nanoparticles comprising paclitaxel coated with an albumin (such as nab-paclitaxel, e.g., ABRAXANE®), wherein the paclitaxel is in the dosage range of about 50-200 mg/m² (such as about 75 mg/m², 100 mg/m², 125 mg/m², or 150 mg/m²), and b) intravenously or intraperitoneally administering to the individual 0.1 to about 15 mg/kg (including for example about 0.1 to about 7 mg/kg or for example about 5, 3, 1, or 0.3 mg/kg) of nivolumab, wherein the nivolumab is intravenously or intraperitoneally administered on day 15 of a 21-day cycle and the nanoparticle composition is administered on day 1 of a 21-day cycle. In some embodiments, the individual suffering from breast cancer (for example, HER2 negative breast cancer or triple negative breast cancer) has not had prior cytotoxic regimens. In some embodiments, the individual suffering from breast cancer (for example, HER2 negative breast cancer or triple negative breast cancer) has had no more than 2 prior cytotoxic regimens. In some embodiments, the individual suffering from breast cancer (for example, HER2 negative breast cancer) has had more than 2 prior cytotoxic regimens. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of nab-paclitaxel (e.g., ABRAXANE®) in the composition is an amount described in Section V-C, infra. In some embodiments, the effective amount of nab-paclitaxel (e.g., ABRAXANE®) in the composition is between about 75 mg/m² to about 275 mg/m². In some embodiments, the effective amount of nab-paclitaxel (e.g., ABRAXANE®) in the composition is between about 80 mg/m² to about 300 mg/m². In some embodiments, the effective amount of nab-paclitaxel (e.g., ABRAXANE®) in the composition is about 80 mg/m² to about 200 mg/m². In some embodiments, the effective amount of nab-paclitaxel (e.g., ABRAXANE®) in the composition is between about 80 mg/m² to about 150 mg/m². In some embodiments, the effective amount of nab-paclitaxel (e.g., ABRAXANE®) in the composition is between about 100 mg/m² to about 250 mg/m². In some embodiments, the effective amount of nab-paclitaxel (e.g., ABRAXANE®) in the composition is between about 170 mg/m² to about 200 mg/m². In some embodiments, the effective amount of nab-paclitaxel (e.g., ABRAXANE®) in the composition is about 50 mg/m². In some embodiments, the effective amount of nab-paclitaxel (e.g., ABRAXANE®) in the composition is about 75 mg/m². In some embodiments, the effective amount of nab-paclitaxel (e.g., ABRAXANE®) in the composition is about 100 mg/m². In some embodiments, the effective amount of nab-paclitaxel (e.g., ABRAXANE®) in the composition is about 125 mg/m². In some embodiments, the effective amount of nab-paclitaxel (e.g., ABRAXANE®) in the composition is about 150 mg/m². In some embodiments, the effective amount of nab-paclitaxel (e.g., ABRAXANE®) in the composition is about 175 mg/m². In some embodiments, the effective amount of nab-paclitaxel (e.g., ABRAXANE®) in the composition is about 200 mg/m². In some embodiments, the effective amount of nab-paclitaxel (e.g., ABRAXANE®) in the composition is about 225 mg/m². In some embodiments, the effective amount of nab-paclitaxel (e.g., ABRAXANE®) in the composition is about 250 mg/m². In some embodiments, the effective amount of nab-paclitaxel (e.g., ABRAXANE®) in the composition is about 260 mg/m². In some embodiments, the effective amount of nab-paclitaxel (e.g., ABRAXANE®) in the composition is about 275 mg/m². In some embodiments, the effective amount of nab-paclitaxel (e.g., ABRAXANE®) in the composition is about 300 mg/m². In some embodiments, the nivolumab is administered to the individual at a dose or dose range described in Section V-C, infra. In some embodiments, the nivolumab is administered to the individual in a dosing schedule as described in Section V-C, infra. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle.

In some embodiments, the breast cancer is a HER2 positive breast cancer. In some embodiments, the breast cancer is a triple negative breast cancer. In some embodiments, the breast cancer is early stage breast cancer, non-metastatic breast cancer, stage IV breast cancer, locally advanced breast cancer, metastatic breast cancer, hormone receptor positive metastatic breast cancer, breast cancer in remission, breast cancer in an adjuvant setting, ductal carcinoma in situ (DCIS), invasive ductal carcinoma (IDC), or breast cancer in a neoadjuvant setting. In some embodiments, the breast cancer is hormone receptor positive metastatic breast cancer. In some embodiments, the breast cancer (which may be HER2 positive or HER2 negative) is advanced breast cancer. In some embodiments, the breast cancer is ductal carcinoma in situ. In some embodiments, the individual may be a human who has a gene, genetic mutation, or polymorphism associated with breast cancer (e.g., BRCA1, BRCA2, ATM, CHEK2, RAD51, AR, DIRAS3, ERBB2, TP53, AKT, PTEN, and/or PI3K) or has one or more extra copies of a gene (e.g., one or more extra copies of the HER2 gene) associated with breast cancer.

In some embodiments, the methods further comprise administration of one or more chemotherapeutic agents.

In some embodiments, there is provided a method of treating a proliferative disease (e.g., cancer) in an individual, comprising administering to the individual: a) an effective amount of a composition comprising nanoparticles comprising a taxane (such as paclitaxel) and a carrier protein (such as albumin), b) an effective amount of a PD-1 pathway antagonist, and c) an effective amount of capecitabine. In some embodiments, there is provided a method of treating a proliferative disease (e.g., cancer) in an individual, comprising administering to the individual: a) an effective amount of a composition comprising nanoparticles comprising a taxane (such as paclitaxel) and a carrier protein (such as albumin), b) an effective amount of a PD-1 pathway antagonist, and c) an effective amount of gemcitabine. In some embodiments, the proliferative disease is a cancer described in Section V-B, infra. In some embodiments, the proliferative disease is a cancer selected from the group consisting of breast cancer, lung cancer (such as small cell lung cancer and non-small cell lung cancer), and pancreatic cancer. In some embodiments, the cancer is breast cancer. In some embodiments, the cancer is a Her2 negative breast cancer. In some embodiments, the cancer is a recurrent breast cancer. In some embodiments, the cancer is a metastatic breast cancer. In some embodiments, the cancer is a lung cancer. In some embodiments, the cancer is a non-small cell lung cancer. In some embodiments, the cancer is a Stage III cancer. In some embodiments, the cancer is a Stage IIIB cancer. In some embodiments, the cancer is a Stage IV cancer. In some embodiments, the cancer is a Stage IIIB or IV NSCLC. In some embodiments, the cancer is pancreatic cancer. In some embodiments, the composition is a nanoparticle composition described in Sections V-C and V-D, infra. In some embodiments, the nanoparticle composition comprises any of the nanoparticle components described in Section V-D1, infra. In some embodiments, the nanoparticle composition is administered in a mode of administration described in Section V-C, infra. In some embodiments, the nanoparticle composition is administered at a dosing frequency described in Section V-C, infra. In some embodiments, the nanoparticle composition is administered in a dosing schedule as described in Section V-C, infra. In some embodiments, the amount of the taxane (such as paclitaxel) in the composition is an amount described in Section V-C, infra. In some embodiments, the effective amount of taxane (e.g., paclitaxel) in the composition is between about 45 mg/m² to about 350 mg/m². In some embodiments, the effective amount of taxane (e.g., paclitaxel) in the composition is about 50 mg/m² to about 300 mg/m². In some embodiments, the effective amount of taxane (e.g., paclitaxel) in the composition is about 50 mg/m² to about 350 mg/m². In some embodiments, the effective amount of taxane (e.g., paclitaxel) in the composition is about 60 mg/m² to about 300 mg/m². In some embodiments, the effective amount of taxane (e.g., paclitaxel) in the composition is between about 75 mg/m² to about 275 mg/m². In some embodiments, the effective amount of taxane (e.g., paclitaxel) in the composition is between about 80 mg/m² to about 350 mg/m². In some embodiments, the effective amount of taxane (e.g., paclitaxel) in the composition is between about 80 mg/m² to about 300 mg/m². In some embodiments, the effective amount of taxane (e.g., paclitaxel) in the composition is between about 80 mg/m² to about 200 mg/m². In some embodiments, the effective amount of taxane (e.g., paclitaxel) in the composition is between about 80 mg/m² to about 150 mg/m². In some embodiments, the effective amount of taxane (e.g., paclitaxel) in the composition is between about 100 mg/m² to about 250 mg/m². In some embodiments, the effective amount of taxane (e.g., paclitaxel) in the composition is between about 150 mg/m² to about 350 mg/m². In some embodiments, the effective amount of taxane (e.g., paclitaxel) in the composition is between about 170 mg/m² to about 200 mg/m². In some embodiments, the effective amount of taxane (e.g., paclitaxel) in the composition is between about 200 mg/m² to about 350 mg/m². In some embodiments, the effective amount of taxane (e.g., paclitaxel) in the composition is about 50 mg/m². In some embodiments, the effective amount of taxane (e.g., paclitaxel) in the composition is about 75 mg/m². In some embodiments, the effective amount of taxane (e.g., paclitaxel) in the composition is about 100 mg/m². In some embodiments, the effective amount of taxane (e.g., paclitaxel) in the composition is about 125 mg/m². In some embodiments, the effective amount of taxane (e.g., paclitaxel) in the composition is about 150 mg/m². In some embodiments, the effective amount of taxane (e.g., paclitaxel) in the composition is about 175 mg/m². In some embodiments, the effective amount of taxane (e.g., paclitaxel) in the composition is about 200 mg/m². In some embodiments, the effective amount of taxane (e.g., paclitaxel) in the composition is about 225 mg/m². In some embodiments, the effective amount of taxane (e.g., paclitaxel) in the composition is about 250 mg/m². In some embodiments, the effective amount of taxane (e.g., paclitaxel) in the composition is about 260 mg/m². In some embodiments, the effective amount of taxane (e.g., paclitaxel) in the composition is about 275 mg/m². In some embodiments, the effective amount of taxane (e.g., paclitaxel) in the composition is about 300 mg/m². In some embodiments, the effective amount of taxane (e.g., paclitaxel) in the composition is about 325 mg/m². In some embodiments, the effective amount of taxane (e.g., paclitaxel) in the composition is about 350 mg/m². In some embodiments, the composition is administered via intravenous administration. In some embodiments, the composition is administered via intra-arterial administration. In some embodiments, the composition is administered via intraperitoneal administration. In some embodiments, the composition is administered via intrapulmonary administration. In some embodiments, the composition is administered via oral administration. In some embodiments, the composition is administered via inhalation. In some embodiments, the composition is administered via intravesicular administration. In some embodiments, the composition is administered via intramuscular administration. In some embodiments, the composition is administered via intra-tracheal administration. In some embodiments, the composition is administered via subcutaneous administration. In some embodiments, the composition is administered via intraocular administration. In some embodiments, the composition is administered via intrathecal administration. In some embodiments, the composition is administered via transmucosal administration. In some embodiments, the composition is administered via transdermal administration. In some embodiments, the effective amount of the composition is administered as a sustained continuous release formulation. In some embodiments, the composition is administered via an inhaler or other air borne delivery system. In some embodiments, the PD-1 pathway antagonist is administered to the individual at a dose or dose range described in Section V-C, infra. In some embodiments, the PD-1 pathway antagonist is administered to the individual in a dosing schedule as described in Section V-C, infra. In some embodiments, the PD-1 pathway antagonist is administered to the individual in a mode of administration described in Section V-C, infra. In some embodiments, the PD-1 pathway antagonist is administered via intravenous administration. In some embodiments, the PD-1 pathway antagonist is administered via intra-arterial administration. In some embodiments, the PD-1 pathway antagonist is administered via intraperitoneal administration. In some embodiments, the PD-1 pathway antagonist is administered via intrapulmonary administration. In some embodiments, the PD-1 pathway antagonist is administered via oral administration. In some embodiments, the PD-1 pathway antagonist is administered via inhalation. In some embodiments, the PD-1 pathway antagonist is administered via intravesicular administration. In some embodiments, the PD-1 pathway antagonist is administered via intramuscular administration. In some embodiments, the PD-1 pathway antagonist is administered via intra-tracheal administration. In some embodiments, the PD-1 pathway antagonist is administered via subcutaneous administration. In some embodiments, the PD-1 pathway antagonist is administered via intraocular administration. In some embodiments, the PD-1 pathway antagonist is administered via intrathecal administration. In some embodiments, the PD-1 pathway antagonist is administered via transmucosal administration. In some embodiments, the PD-1 pathway antagonist is administered via transdermal administration. In some embodiments, the effective amount of the PD-1 pathway antagonist is administered as a sustained continuous release formulation. In some embodiments, the PD-1 pathway antagonist is administered via an inhaler or other air borne delivery system. In some embodiments, the PD-1 pathway antagonist is selected from the group consisting of AMP-224, BMS-936559, MEDI4736, MSB0010718C MPDL3280A, nivolumab, AMP-514, pembrolizumab (MK-3475), REGN2810, PDR001, BGB-A317, and pidilizumab (CT-011). In some embodiments, the PD-1 pathway antagonist is an antagonist of PD-1. In some embodiments, the PD-1 pathway antagonist is an anti-PD-1 antibody. In some embodiments, the PD-1 pathway antagonist is nivolumab. In some embodiments, the PD-1 pathway antagonist is an antagonist of a ligand of PD-1. In some embodiments, the PD-1 pathway antagonist is an antagonist of PD-L1. In some embodiments, the PD-1 pathway antagonist is an anti-PD-L1 antibody. In some embodiments, the PD-1 pathway antagonist is BMS-936559. In some embodiments, BMS-936559 is administered to the individual at a dose or dose range described in Section V-C, infra. In some embodiments, BMS-936559 is administered to the individual in a dosing schedule as described in Section V-C, infra. In some embodiments, BMS-936559 is administered to the individual in a mode of administration described in Section V-C, infra. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle. In some embodiments, the PD-1 pathway antagonist is MPDL3280A. In some embodiments, MPDL3280A is administered to the individual at a dose or dose range described in Section V-C, infra. In some embodiments, MPDL3280A is administered to the individual in a dosing schedule as described in Section V-C, infra. In some embodiments, MPDL3280A is administered to the individual in a mode of administration described in Section V-C, infra. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle. In some embodiments, the PD-1 pathway antagonist is MEDI4736. In some embodiments, MEDI4736 is administered to the individual at a dose or dose range described in Section V-C, infra. In some embodiments, MEDI4736 is administered to the individual in a dosing schedule as described in Section V-C, infra. In some embodiments, MEDI4736 is administered to the individual in a mode of administration described in Section V-C, infra. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle. In some embodiments, the PD-1 pathway antagonist is MSB0010718C. In some embodiments, MSB0010718C is administered to the individual at a dose or dose range described in Section V-C, infra. In some embodiments, MSB0010718C is administered to the individual in a dosing schedule as described in Section V-C, infra. In some embodiments, MSB0010718C is administered to the individual in a mode of administration described in Section V-C, infra. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle. In some embodiments, the PD-1 pathway antagonist is an antagonist of PD-L2. In some embodiments, the PD-1 pathway antagonist is an anti-PD-L2 antibody. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In some embodiments of any of the methods described herein, the taxane is paclitaxel. In some embodiments of any of the methods described herein, the average diameter of the nanoparticles in the composition is no greater than about 200 nm. In some embodiments, the diameter of the nanoparticle is determined by laser diffraction or light scattering. In some embodiments of any of the methods described herein, the carrier protein is albumin. In some embodiments, the weight ratio of the carrier protein (e.g., albumin) to the taxane (e.g., paclitaxel) in the nanoparticle composition is between about 1:1 to about 18:1. In some embodiments, the weight ratio of the carrier protein (e.g., albumin) to the taxane (e.g., paclitaxel) in the nanoparticle composition is between about 1:1 to about 9:1. In some embodiments, the weight ratio of the carrier protein (e.g., albumin) to the taxane (e.g., paclitaxel) in the nanoparticle composition 9:1. In some embodiments, the nanoparticle has an average diameter of less than or equal to about 200 nm and the weight ratio of the carrier protein (such as albumin) to taxane (such as paclitaxel) in the composition is between about 1:1 to about 9:1. In some embodiments, the nanoparticle has an average diameter of less than or equal to about 200 nm and the weight ratio of the carrier protein (such as albumin) to taxane (such as paclitaxel) in the composition is about 9:1. In some embodiments of any of the methods described above, the individual is a human.

The present application also provides pharmaceutical compositions comprising nanoparticles comprising a taxane (such as paclitaxel) and a carrier protein (such as albumin) for use in the treatment of a proliferative disease (such as cancer), wherein said use comprises simultaneous, sequential, and/or concurrent administration of an agent that antagonizes a PD-1 pathway in a cell. In some embodiments, provided herein is a pharmaceutical composition comprising an agent that antagonizes a PD-1 pathway in a cell for use in the treatment of a proliferative disease (such as cancer), wherein said use comprises simultaneous, sequential, and/or concurrent administration of a composition comprising nanoparticles comprising a taxane (such as paclitaxel) and a carrier protein (such as albumin). In some embodiments, provided herein are taxane-containing nanoparticle compositions and compositions comprising an agent that inhibits prosurvival and/or inflammatory signal for simultaneous, sequential, and/or concurrent use for treatment of a proliferative disease (such as cancer).

In some embodiments, there is provided a kit comprising: a) a composition comprising nanoparticles comprising a taxane (such as paclitaxel) and a carrier protein (such as albumin), and b) an effective amount of at least one other agent that antagonizes a PD-1 pathway in a cell. In some embodiments, there is provided a pharmaceutical composition comprising: a) a composition comprising nanoparticles comprising a taxane (such as paclitaxel) and a carrier protein (such as albumin), and b) an effective amount of at least one other agent that antagonizes a PD-1 pathway in a cell.

### Section V-B. Methods of Treating Proliferative Diseases

The combination therapy methods described herein are useful for treating proliferative diseases. The methods require administration of the nanoparticle composition and the other agent in effective amounts. In some embodiments, an effective amount is an amount sufficient to delay development. In some embodiments, an effective amount is an amount sufficient to prevent or delay recurrence. An effective amount can be administered in one or more administrations. In the case of cancer, the effective amount of the drug or composition may: (i) reduce the number of cancer cells; (ii) reduce tumor size; (iii) reduce tumor burden; (iv) inhibit, retard, slow to some extent and preferably stop cancer cell infiltration into peripheral organs; (v) inhibit (i.e., slow to some extent and preferably stop) tumor metastasis; (vi) inhibit tumor growth; (vii) reduce the rate of tumor growth; (viii) prevent or delay occurrence and/or recurrence of tumor; (ix) relieve to some extent one or more of the symptoms associated with the cancer; (x) prolong survival; and/or (xi) prolong progression-free survival.

Thus, in some embodiments, there is provided a method of inhibiting cell proliferation (such as tumor growth) in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the taxane is paclitaxel. In some embodiments, the composition comprises nab-paclitaxel, e.g., ABRAXANE®. In some embodiments, the effective amounts of the taxane nanoparticle composition and the other agent synergistically inhibit cell proliferation (such as tumor cell growth). In some embodiments, the effective amounts of the taxane nanoparticle composition and the other agent inhibit cell proliferation (such as tumor cell growth) to a greater degree than the existing standard of care. In some embodiments, at least about 10% (including for example at least about any of 20%, 30%, 40%, 60%, 70%, 80%, 90%, or 100%) cell proliferation is inhibited. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, pidilizumab (CT-011), and rHIgM12B7. In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by oral administration. In some embodiments, the other agent is administered intravenously or subcutaneously. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1.

In some embodiments, there is provided a method of inhibiting tumor metastasis (such as metastasis of breast cancer, metastasis of pancreatic cancer, pulmonary metastasis or metastasis to the lymph node) in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the taxane is paclitaxel. In some embodiments, the composition comprises nab-paclitaxel, e.g, ABRAXANE®. In some embodiments, the effective amounts of the taxane nanoparticle composition and the other agent synergistically inhibit tumor metastasis. In some embodiments, the effective amounts of the taxane nanoparticle composition and the other agent inhibit tumor metastasis to a greater degree than the existing standard of care. In some embodiments, at least about 10% (including for example at least about any of 20%, 30%, 40%, 60%, 70%, 80%, 90%, or 100%) metastasis is inhibited. In some embodiments, method of inhibiting metastasis to lymph node is provided. In some embodiments, method of inhibiting metastasis to the lung is provided. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, pidilizumab (CT-011), and rHIgM12B7. In some embodiments, the other agent is nivolumab. In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is an antagonist of PD-L2, for example, rHIgM12B7. In some embodiments, the composition comprising nanoparticles is administered by intravenous administration. In some embodiments, the other agent is administered by oral administration. In some embodiments, the other agent is administered intravenously or subcutaneously. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1.

In some embodiments, there is provided a method of reducing (such as eradicating) pre-existing tumor metastasis (such as breast cancer metastasis, or pulmonary metastasis) in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the taxane is paclitaxel. In some embodiments, the composition comprises nab-paclitaxel, e.g., ABRAXANE®. In some embodiments, the effective amounts of the taxane nanoparticle composition and the other agent synergistically reduces (such as eradicates) tumor metastasis. In some embodiments, the effective amounts of the taxane nanoparticle composition and the other agent reduce tumor metastasis to a greater degree than the existing standard of care. In some embodiments, at least about 10% (including for example at least about any of 20%, 30%, 40%, 60%, 70%, 80%, 90%, or 100%) metastasis is reduced. In some embodiments, method of reducing metastasis to lymph node is provided. In some embodiments, method of reducing metastasis to the lung is provided. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, and pidilizumab (CT-011). In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by oral administration. In some embodiments, the other agent is administered intravenously or subcutaneously. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1.

In some embodiments, there is provided a method of reducing incidence or burden of preexisting tumor metastasis (such as pulmonary metastasis or metastasis to the lymph node) in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by oral administration. In some embodiments, the other agent is administered intravenously or subcutaneously.

In some embodiments, there is provided a method of reducing tumor size in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the taxane is paclitaxel. In some embodiments, the composition comprises nab-paclitaxel, e.g., ABRAXANE®. In some embodiments, the effective amounts of the taxane nanoparticle composition and the other agent synergistically reduces tumor size. In some embodiments, the effective amounts of the taxane nanoparticle composition and the other agent reduce tumor size to a greater degree than the existing standard of care. In some embodiments, the tumor size is reduced at least about 10% (including for example at least about any of 20%, 30%, 40%, 60%, 70%, 80%, 90%, or 100%). In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by intraperitoneal administration. In some embodiments, the other agent is administered by oral administration. In some embodiments, the other agent is administered intravenously or subcutaneously. In some embodiments, the weight ratio of the carrier protein (such as albumin) to taxane (such as paclitaxel) in the composition is between about 1:1 to about 18:1. In some embodiments, the weight ratio of the carrier protein (such as albumin) to taxane (such as paclitaxel) in the composition is between about 1:1 to about 9:1. In some embodiments, the weight ratio of the carrier protein (such as albumin) to taxane (such as paclitaxel) in the composition is about 9:1. In some embodiments, the nanoparticle has an average diameter of less than or equal to about 200 nm and the weight ratio of the carrier protein (such as albumin) to taxane (such as paclitaxel) in the composition is between about 1:1 to about 18:1. In some embodiments, the nanoparticle has an average diameter of less than or equal to about 200 nm and the weight ratio of the carrier protein (such as albumin) to taxane (such as paclitaxel) in the composition is between about 1:1 to about 9:1.

In some embodiments, there is provided a method of prolonging time to disease progression of a proliferative disease (such as cancer) in an individual, comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the method prolongs the time to disease progression by at least any of 1, 2, 3, 4, 5, 6, 7, 8, 9, 10, 11, or 12 weeks. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by intraperitoneal administration. In some embodiments, the other agent is administered by oral administration. In some embodiments, the other agent is administered intravenously or subcutaneously.

In some embodiments, there is provided a method of prolonging survival of an individual having a proliferative disease (such as cancer), comprising administering to the individual: a) an effective amount of a composition comprising nanoparticles comprising a taxane (such as paclitaxel) and a carrier protein (such as albumin), and b) an effective amount of at least one other agent that antagonizes a PD-1 pathway in a cell. In some embodiments, the method prolongs the survival of the individual by at least any of 1, 2, 3, 4, 5, 6, 7, 8, 9, 10, 11, 12, 18, or 24 month. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by intraperitoneal administration. In some embodiments, the other agent is administered by oral administration. In some embodiments, the other agent is administered intravenously or subcutaneously.

It is understood that any of the embodiments in this section apply to the embodiments provided in the section “methods of combination therapy.” For example, in some embodiments, there is provided a method of reducing (such as eradicating) pre-existing tumor metastasis (such as pulmonary metastasis or metastasis to the lymph node) in an individual, comprising administering to the individual: a) an effective amount of a composition comprising nanoparticles comprising taxane and a carrier protein (such as albumin), and b) an effective amount of an agent that antagonizes a PD-1 pathway in a cell, wherein the nanoparticle composition and the agent that antagonizes a PD-1 pathway in a cell are administered concurrently. In some embodiments, there is provided a method of reducing (such as eradicating) pre-existing tumor metastasis (such as pulmonary metastasis or metastasis to the lymph node) in an individual, comprising administering to the individual: a) an effective amount of nanoparticles comprising paclitaxel coated with albumin (such as nab-paclitaxel, e.g., ABRAXANE®), and b) an effective amount of an agent that antagonizes a PD-1 pathway in a cell, wherein the nanoparticle composition and the agent that antagonizes a PD-1 pathway in a cell are administered concurrently. In some embodiments, there is provided a method of reducing (such as eradicating) pre-existing tumor metastasis (such as pulmonary metastasis or metastasis to the lymph node) in an individual, comprising administering to the individual: a) an effective amount of a composition comprising nanoparticles comprising a taxane (such as paclitaxel) and a carrier protein (such as albumin), and b) an effective amount of an agent that antagonizes a PD-1 pathway in a cell, wherein the nanoparticle composition and the agent that antagonizes a PD-1 pathway in a cell are administered sequentially. In some embodiments, there is provided a method of reducing (such as eradicating) pre-existing tumor metastasis (such as pulmonary metastasis or metastasis to the lymph node) in an individual, comprising administering to the individual: a) an effective amount of nanoparticles comprising paclitaxel coated with albumin (such as nab-paclitaxel, e.g., ABRAXANE®), and b) an effective amount of an agent that antagonizes a PD-1 pathway in a cell, wherein the nanoparticle composition and the agent that antagonizes a PD-1 pathway in a cell are administered sequentially. In some embodiments, the agent that antagonizes a PD-1 pathway in a cell is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7.

The effectiveness of the methods presented herein can be assessed by one or more criteria, which include, but are not limited to, markers of proliferation and/or apoptosis, gene methylation, gene expression profile, and markers of immune responses (e.g., cytokines). In some embodiments, the effectiveness of the method can be assessed by functional imaging, such as PET/CT scans and/or fludeoxyglucose F 18-position emission tomography (FDG-PET) (Sun, X. et al. J. Nucl. Med. (2011) 52(1):140-146).

Thus, for example, in some embodiments, there is provided a method of treating a proliferative disease (such as cancer) in an individual, comprising administering to the individual a) an effective amount of a composition comprising nanoparticles comprising a taxane (such as paclitaxel) and a carrier protein (such as albumin), and b) an effective amount of at least one other agent that antagonizes a PD-1 pathway in a cell, wherein the standard uptake value (SUV) determined in a FDG-PET scan in the individual is decreased by at least about any of 10%, 20%, 30%, 40%, 50%, 60%, 70%, 80%, 90%, or 95%. In some embodiments, the taxane is paclitaxel. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is AMP-224. In some embodiments, the other agent is a PD-1 antagonist. In some embodiments, the PD-1 antagonist is selected from the group consisting of nivolumab (BMS-936558), AMP-514, pembrolizumab (MK-3475), REGN2810, PDR001, BGB-A317, and pidilizumab (CT-011). In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is a PD-L1 antagonist. In some embodiments, the PD-L1 antagonist is selected from the group consisting of BMS-936559, MEDI4736, MSB0010718C and MPDL3280A (RG7446)). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is an antagonist of PD-L2, for example, rHIgM12B7. In some embodiments, the composition comprising nanoparticles is administered by intravenous administration. In some embodiments, the other agent is administered by intraperitoneal administration. In some embodiments, the other agent is administered by oral administration. In some embodiments, the method further comprises determining a baseline SUV value in the individual prior to the treatment. In some embodiments, the method further comprises determining the SUV value in the individual after the treatment.

The treatment methods can also be evaluated for safety and toxicity, for example, based on NCI CTCAE analyses.

In some embodiments, the proliferative disease is cancer. In particular embodiments, the cancer is breast cancer. These methods can be used, for example, to treat, stabilize, prevent, and/or delay any type or stage of breast cancer, such as early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some embodiments, the method is useful for preoperative systemic therapy (PST).

In some embodiments, there is provided a method of treating breast cancer (which may, for example, be HER2 positive, HER2 negative or triple negative), including, for example, advanced breast cancer, stage IV breast cancer, locally advanced breast cancer, and metastatic breast cancer. In some embodiments, the breast cancer is luminal type B breast cancer. In some embodiments, the breast cancer is basal cell breast cancer. In some embodiments, the individual is diagnosed with T2, T3, or T4 lesion, or a stage N, M0 or T1c, N1-3 and M0. In some embodiments, the individual has an ECOG performance status of 0-1. In some embodiments, the individual has skin metastasis to the ipsilateral breast. In some embodiments, the individual has undergone prior therapy (such as hormonal therapy). In some embodiments, the individual has not undergone prior therapy (such as hormonal therapy). In some embodiments, the individual is awaiting definitive surgery. In some embodiments, the breast cancer is resected breast cancer. In some embodiments, the breast cancer is unresected breast cancer, such as unresected stage II or III breast cancer.

In some embodiments, the method is for treating an individual having one or more of these risk factors resulting in a higher probability of developing breast cancer than an individual without these risk factor(s). These risk factors include, but are not limited to, age, sex, race, diet, history of previous disease, presence of precursor disease, genetic (i.e., hereditary) considerations, and environmental exposure. In some embodiments, the individual may be a human who is genetically or otherwise predisposed to developing breast cancer who has or has not been diagnosed with breast cancer. Individuals at risk for breast cancer include, e.g., those having relatives who have experienced this disease, and those whose risk is determined by analysis of genetic or biochemical markers. For example, the individual may be a human who has a gene, genetic mutation, or polymorphism associated with breast cancer (e.g., BRCA1, BRCA2, ATM, CHEK2, RAD51, AR, DIRAS3, ERBB2, and/or TP53) or has one or more extra copies of a gene (e.g., one or more extra copies of the HER2 gene) associated with breast cancer. In some embodiments, the breast cancer is HER2 negative. In some embodiments, the breast cancer is ER negative. In some embodiments, the breast cancer is PR negative. In some embodiments, the breast cancer is ER negative and HER2 negative. In some embodiments, the breast cancer is PR negative and HER2 negative. In some embodiments, the breast cancer is ER negative and PR negative. In some embodiment, the breast cancer is ER negative, PR negative, and HER2 negative.

In some embodiments, the cancer is lung cancer, including, for example, non-small cell lung cancer (NSCLC) such as advanced NSCLC, small cell lung cancer (SCLC), such as advanced SCLC, and advanced solid tumor malignancy in the lung.

In some embodiments, the cancer is pancreatic cancer, including for example pancreatic adenocarcinoma, pancreatic adenosquamous carcinoma, pancreatic squamous cell carcinoma, and pancreatic giant cell carcinoma. In some embodiments, the pancreatic cancer is exocrine pancreatic cancer. In some embodiments, the pancreatic cancer is endocrine pancreatic cancer (such as islet cell carcinoma). In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

### Section V-C. Modes of Administration

The composition comprising nanoparticles comprising a taxane (also referred to as “nanoparticle composition”) and the other agent can be administered simultaneously (i.e., simultaneous administration) and/or sequentially (i.e., sequential administration).

In some embodiments, the nanoparticle composition and the other agent (including the specific agents described herein) are administered simultaneously. The term “simultaneous administration,” as used herein, means that the nanoparticle composition and the other agent are administered with a time separation of no more than about 15 minute(s), such as no more than about any of 10, 5, or 1 minutes. When the drugs are administered simultaneously, the drug in the nanoparticles and the other agent may be contained in the same composition (e.g., a composition comprising both the nanoparticles and the other agent) or in separate compositions (e.g., the nanoparticles are contained in one composition and the other agent is contained in another composition).

In some embodiments, the nanoparticle composition and the other agent are administered sequentially. The term “sequential administration” as used herein means that the drug in the nanoparticle composition and the other agent are administered with a time separation of more than about 15 minutes, such as more than about any of 20, 30, 40, 50, 60 or more minutes. Either the nanoparticle composition or the other agent may be administered first. The nanoparticle composition and the other agent are contained in separate compositions, which may be contained in the same or different packages.

In some embodiments, the administration of the nanoparticle composition and the other agent are concurrent, i.e., the administration period of the nanoparticle composition and that of the other agent overlap with each other. In some embodiments, the nanoparticle composition is administered for at least one cycle (for example, at least any of 2, 3, or 4 cycles) prior to the administration of the other agent. In some embodiments, the other agent is administered for at least any of one, two, three, or four weeks. In some embodiments, the administrations of the nanoparticle composition and the other agent are initiated at about the same time (for example, within any one of 1, 2, 3, 4, 5, 6, or 7 days). In some embodiments, the administrations of the nanoparticle composition and the other agent are terminated at about the same time (for example, within any one of 1, 2, 3, 4, 5, 6, or 7 days). In some embodiments, the administration of the other agent continues (for example for about any one of 1, 2, 3, 4, 5, 6, 7, 8, 9, 10, 11, or 12 months) after the termination of the administration of the nanoparticle composition. In some embodiments, the administration of the other agent is initiated after (for example after about any one of 1, 2, 3, 4, 5, 6, 7, 8, 9, 10, 11, or we months) the initiation of the administration of the nanoparticle composition. In some embodiments, the administrations of the nanoparticle composition and the other agent are initiated and terminated at about the same time. In some embodiments, the administrations of the nanoparticle composition and the other agent are initiated at about the same time and the administration of the other agent continues (for example for about any one of 1, 2, 3, 4, 5, 6, 7, 8, 9, 10, 11, or 12 months) after the termination of the administration of the nanoparticle composition. In some embodiments, the administration of the nanoparticle composition and the other agent stop at about the same time and the administration of the other agent is initiated after (for example after about any one of 1, 2, 3, 4, 5, 6, 7, 8, 9, 10, 11, or we months) the initiation of the administration of the nanoparticle composition.

In some embodiments, the administration of the nanoparticle composition and the other agent are non-concurrent. For example, in some embodiments, the administration of the nanoparticle composition is terminated before the other agent is administered. In some embodiments, the administration of the other agent is terminated before the nanoparticle composition is administered. The time period between these two non-concurrent administrations can range from about two to eight weeks, such as about four weeks.

The dosing frequency of the drug-containing nanoparticle composition and the other agent may be adjusted over the course of the treatment, based on the judgment of the administering physician. When administered separately, the drug-containing nanoparticle composition and the other agent can be administered at different dosing frequency or intervals. For example, the drug-containing nanoparticle composition can be administered weekly, while another agent can be administered more or less frequently. In some embodiments, sustained continuous release formulation of the drug-containing nanoparticle and/or other agent may be used. Various formulations and devices for achieving sustained release are known in the art. Exemplary dosing frequencies are further provided herein.

The nanoparticle composition and the other agent can be administered using the same route of administration or different routes of administration. Exemplary administration routes are further provided herein. In some embodiments (for both simultaneous and sequential administrations), the taxane in the nanoparticle composition and the other agent are administered at a predetermined ratio. For example, in some embodiments, the ratio by weight of the taxane in the nanoparticle composition and the other agent is about 1 to 1. In some embodiments, the weight ratio may be between about 0.001 to about 1 and about 1000 to about 1, or between about 0.01 to about 1 and 100 to about 1. In some embodiments, the ratio by weight of the taxane in the nanoparticle composition and the other agent is less than about any of 100:1, 50:1, 30:1, 10:1, 9:1, 8:1, 7:1, 6:1, 5:1, 4:1, 3:1, 2:1, and 1:1 In some embodiments, the ratio by weight of the taxane in the nanoparticle composition and the other agent is more than about any of 1:1, 2:1, 3:1, 4:1, 5:1, 6:1, 7:1, 8:1, 9:1, 30:1, 50:1, 100:1. Other ratios are contemplated.

The doses required for the taxane and/or the other agent may (but not necessarily) be lower than what is normally required when each agent is administered alone. Thus, in some embodiments, a subtherapeutic amount of the drug in the nanoparticle composition and/or the other agent are administered. “Subtherapeutic amount” or “subtherapeutic level” refer to an amount that is less than therapeutic amount, that is, less than the amount normally used when the drug in the nanoparticle composition and/or the other agent are administered alone. The reduction may be reflected in terms of the amount administered at a given administration and/or the amount administered over a given period of time (reduced frequency).

In some embodiments, enough other agent is administered so as to allow reduction of the normal dose of the drug in the nanoparticle composition required to effect the same degree of treatment by at least about any of 5%, 10%, 20%, 30%, 50%, 60%, 70%, 80%, 90%, or more. In some embodiments, enough drug in the nanoparticle composition is administered so as to allow reduction of the normal dose of the other agent required to effect the same degree of treatment by at least about any of 5%, 10%, 20%, 30%, 50%, 60%, 70%, 80%, 90%, or more.

In some embodiments, the dose of both the taxane in the nanoparticle composition and the other agent are reduced as compared to the corresponding normal dose of each when administered alone. In some embodiments, both the taxane in the nanoparticle composition and the other agent are administered at a subtherapeutic, i.e., reduced, level. In some embodiments, the dose of the nanoparticle composition and/or the other agent is substantially less than the established maximum toxic dose (MTD). For example, the dose of the nanoparticle composition and/or the other agent is less than about 50%, 40%, 30%, 20%, or 10% of the MTD.

In some embodiments, the dose of taxane and/or the dose of the other agent is higher than what is normally required when each agent is administered alone. For example, in some embodiments, the dose of the nanoparticle composition and/or the other agent is substantially higher than the established maximum toxic dose (MTD). For example, the dose of the nanoparticle composition and/or the other agent is more than about 50%, 40%, 30%, 20%, or 10% of the MTD of the agent when administered alone.

In some embodiments, the amount of a taxane (e.g., paclitaxel) in the composition is included in any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of a taxane (e.g., paclitaxel) in the effective amount of the composition (e.g., a unit dosage form) is in the range of about 5 mg to about 500 mg, such as about 30 mg to about 300 mg or about 50 mg to about 200 mg. In some embodiments, the concentration of the taxane (e.g., paclitaxel) in the composition is dilute (about 0.1 mg/ml) or concentrated (about 100 mg/ml), including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane (e.g., paclitaxel) is at least about any of 0.5 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, or 50 mg/ml.

Exemplary effective amounts of a taxane (e.g., paclitaxel) in the nanoparticle composition include, but are not limited to, at least about any of 25 mg/m², 30 mg/m², 50 mg/m², 60 mg/m², 75 mg/m², 80 mg/m², 90 mg/m², 100 mg/m², 120 mg/m², 125 mg/m², 150 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² of a taxane (e.g., paclitaxel). In various embodiments, the composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² of a taxane (e.g., paclitaxel). In some embodiments, the amount of the taxane (e.g., paclitaxel) per administration is less than about any of 25 mg/m², 22 mg/m², 20 mg/m², 18 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the effective amount of a taxane (e.g., paclitaxel) in the composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the effective amount of a taxane (e.g., paclitaxel) in the composition is about 5 to about 300 mg/m², such as about 20 to about 300 mg/m², about 50 to about 250 mg/m², about 100 to about 150 mg/m², about 120 mg/m², about 130 mg/m², or about 140 mg/m², or about 260 mg/m².

In some embodiments of any of the above aspects, the effective amount of a taxane (e.g., paclitaxel) in the composition includes at least about any of 1 mg/kg, 2.5 mg/kg, 3.5 mg/kg, 5 mg/kg, 6.5 mg/kg, 7.5 mg/kg, 10 mg/kg, 15 mg/kg, or 20 mg/kg. In various embodiments, the effective amount of a taxane (e.g., paclitaxel) in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg of a taxane (e.g., paclitaxel).

Exemplary dosing frequencies for the nanoparticle composition (and as indicated below for the other agent) include, but are not limited to, weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week, or three times daily, two times daily. In some embodiments, the intervals between each administration are less than about any of 6 months, 3 months, 1 month, 20 days, 15 days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the taxane in the nanoparticle composition is administered weekly. In some embodiments, the taxane in a nanoparticle composition is administered every two weeks. In some embodiments, the taxane in the nanoparticle composition is administered every three weeks. In some embodiments, the other agent is administered l×, 2×, 3×, 4×, 5×, 6×, or 7 times a week. In some embodiments, the other agent is administered every two weeks or two out of three weeks. In some embodiments, the other agent is administered every three weeks or three out of four weeks. In some embodiments, the taxane is paclitaxel. In some embodiment, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-0111), and rHIgM12B7. In some embodiments of the above dosages and/or administrations, the taxane is paclitaxel and the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-0111), and rHIgM12B7. In some embodiments, the other agent is a PD-1 antagonist. In some embodiments, the other agent is nivolumab.

The administration of the nanoparticle composition (and for the other agent) can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months. In some embodiments, the taxane (e.g., paclitaxel) is administered over a period of at least one month, wherein the interval between each administration is no more than about a week, and wherein the dose of the taxane (e.g., paclitaxel) at each administration is about 0.25 mg/m² to about 75 mg/m², such as about 0.25 mg/m² to about 25 mg/m² or about 25 mg/m² to about 50 mg/m².

In some embodiments, the dosage of a taxane (e.g., paclitaxel) in a nanoparticle composition can be in the range of 5-400 mg/m² when given on a 3 week schedule, or 5-250 mg/m² when given on a weekly schedule. For example, the amount of a taxane (e.g., paclitaxel) can be about 60 to about 300 mg/m² (e.g., about 260 mg/m²) when given on a three week schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of four weeks; 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m², twice a week; and 150-250 mg/m² twice a week. The dosing frequency of the composition may be adjusted over the course of the treatment based on the judgment of the administering physician.

In some embodiments, the individual is treated for at least about any of one, two, three, four, five, six, seven, eight, nine, or ten treatment cycles. The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary dose of the taxane (in some embodiments paclitaxel) in the nanoparticle composition include, but is not limited to, about any of 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of taxane (e.g., paclitaxel) in a nanoparticle composition can be in the range of about 100-400 mg/m² when given on a 3 week schedule, or about 50-250 mg/m² when given on a weekly schedule.

The dosing frequency of the other agent can be the same or different from that of the nanoparticle composition. Exemplary frequencies are provided herein. As further example, the other agent can be administered three times a day, two times a day, daily, 6 times a week, 5 times a week, 4 times a week, 3 times a week, two times a week, weekly. In some embodiments, the other agent is administered twice daily or three times daily. Exemplary amounts of the other agent include, but are not limited to, any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg, about 500 to about 550 mg, about 550 to about 600 mg, about 600 to about 650 mg, about 650 to about 700 mg, about 700 mg to about 800 mg, about 800 mg to about 850 mg, about 850 mg to about 900 mg, about 900 mg to about 950 mg, about 950 mg to about 1000 mg. For example, the other agent can be administered at a dose of about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the other agent is a PD-1 antagonist. In some embodiments, the other agent is an anti-PD-1 antibody. In some embodiments, the other agent is an antagonist of a PD-1 ligand. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the anti-PD-1 antibody is selected from the group consisting of nivolumab, AMP-514, pembrolizumab (MK-3475), REGN2810, PDR001, BGB-A317, and pidilizumab (CT-011). In some embodiments, the antagonist of a PD-1 ligand is selected from the group consisting of AMP-224, BMS-936559, MEDI4736, MSB0010718C and MPDL3280A. In some embodiments, the other agent is nivolumab. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, nivolumab (BMS-936558), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, nivolumab (BMS-936558) is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the nivolumab is administered as an intravenous infusion over about 10, 20, 30, 40, 50, or 60 or more minutes. In some embodiments, the nivolumab is administered as an intravenous infusion over about 60 minutes once every 1, 2, 3, 4, 5 or more weeks. In some embodiments, the nivolumab is administered as an intravenous infusion over about 60 minutes once every two weeks. In some embodiments, the nivolumab is administered as an intravenous infusion over about 60 minutes once every three weeks. In some embodiments, the nivolumab is administered as an intravenous infusion over about 60 minutes once every four weeks. In some embodiments, the other agent is pembrolizumab. For example, in some embodiments, pembrolizumab is administered at about 0.5 to about 20 mg/kg (including for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg). For example, in some embodiments, pembrolizumab is administered at about 0.5 to about 20 mg/kg (including for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) once a week. For example, in some embodiments, pembrolizumab is administered at about 0.5 to about 20 mg/kg (including for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) once every two weeks. For example, in some embodiments, pembrolizumab is administered at about 0.5 to about 20 mg/kg (including for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) once every three weeks. For example, in some embodiments, pembrolizumab is administered at about 0.5 to about 20 mg/kg (including for example, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg) once every four weeks. In some embodiments, the pembrolizumab is administered as an intravenous infusion over about 10, 20, 30, 40, 50, or 60 or more minutes. In some embodiments, the pembrolizumab is administered as an intravenous infusion over about 60 minutes once every 1, 2, 3, 4, 5 or more weeks. In some embodiments, the pembrolizumab is administered as an intravenous infusion over about 60 minutes once every two weeks. In some embodiments, the pembrolizumab is administered as an intravenous infusion over about 60 minutes once every three weeks. In some embodiments, the pembrolizumab is administered as an intravenous infusion over about 60 minutes once every four weeks. In some embodiments, the other agent is pidilizumab. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, pidilizumab (CT-011), is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, pidilizumab (CT-011) is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the pidilizumab (CT-011) is administered as an intravenous infusion over about 10, 20, 30, 40, 50, or 60 or more minutes. In some embodiments, the pidilizumab (CT-011) is administered as an intravenous infusion over about 60 minutes once every 1, 2, 3, 4, 5 or more weeks. In some embodiments, the pidilizumab (CT-011) is administered as an intravenous infusion over about 60 minutes once every two weeks. In some embodiments, the pidilizumab (CT-011) is administered as an intravenous infusion over about 60 minutes once every three weeks. In some embodiments, the pidilizumab (CT-011) is administered as an intravenous infusion over about 60 minutes once every four weeks. In some embodiments, the other agent is REGN2810. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, REGN2810, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, REGN2810 is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the REGN2810 is administered as an intravenous infusion over about 10, 20, 30, 40, 50, or 60 or more minutes. In some embodiments, the REGN2810 is administered as an intravenous infusion over about 60 minutes once every 1, 2, 3, 4, 5 or more weeks. In some embodiments, the REGN2810 is administered as an intravenous infusion over about 60 minutes once every two weeks. In some embodiments, the REGN2810 is administered as an intravenous infusion over about 60 minutes once every three weeks. In some embodiments, the REGN2810 is administered as an intravenous infusion over about 60 minutes once every four weeks. In some embodiments, the other agent is PDR001. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, PDR001, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, PDR001 is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the PDR001 is administered as an intravenous infusion over about 10, 20, 30, 40, 50, or 60 or more minutes. In some embodiments, the PDR001 is administered as an intravenous infusion over about 60 minutes once every 1, 2, 3, 4, 5 or more weeks. In some embodiments, the PDR001 is administered as an intravenous infusion over about 60 minutes once every two weeks. In some embodiments, the PDR001 is administered as an intravenous infusion over about 60 minutes once every three weeks. In some embodiments, the PDR001 is administered as an intravenous infusion over about 60 minutes once every four weeks. In some embodiments, the other agent is BGB-A317. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, BGB-A317, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, BGB-A317 is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the BGB-A317 is administered as an intravenous infusion over about 10, 20, 30, 40, 50, or 60 or more minutes. In some embodiments, the BGB-A317 is administered as an intravenous infusion over about 60 minutes once every 1, 2, 3, 4, 5 or more weeks. In some embodiments, the BGB-A317 is administered as an intravenous infusion over about 60 minutes once every two weeks. In some embodiments, the BGB-A317 is administered as an intravenous infusion over about 60 minutes once every three weeks. In some embodiments, the BGB-A317 is administered as an intravenous infusion over about 60 minutes once every four weeks. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), every two weeks. In some embodiments, the other agent is AMP-514. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 4 weeks. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every four weeks. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every three weeks. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 21 days. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 21 days. For example, in some embodiments, AMP-514, is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), once every 28 days. For example, in some embodiments, AMP-514 is administered (for example by intravenous infusion) at about 0.1 to about 10 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg), twice every 28 days. In some embodiments, the AMP-514 is administered as an intravenous infusion over about 10, 20, 30, 40, 50, or 60 or more minutes. In some embodiments, the AMP-514 is administered as an intravenous infusion over about 60 minutes once every 1, 2, 3, 4, 5 or more weeks. In some embodiments, the AMP-514 is administered as an intravenous infusion over about 60 minutes once every two weeks. In some embodiments, the AMP-514 is administered as an intravenous infusion over about 60 minutes once every three weeks. In some embodiments, the AMP-514 is administered as an intravenous infusion over about 60 minutes once every four weeks. In some embodiments, the other agent is AMP-224. For example, in some embodiments, AMP-224, is administered (for example by intravenous infusion) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), every two weeks. For example, in some embodiments, AMP-224, is administered (for example by intravenous infusion) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), once every 4 weeks. For example, in some embodiments, AMP-224, is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), twice every four weeks. For example, in some embodiments, AMP-224, is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), once every three weeks. For example, in some embodiments, AMP-224, is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), once every 21 days. For example, in some embodiments, AMP-224, is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), twice every 21 days. For example, in some embodiments, AMP-224, is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), once every 28 days. For example, in some embodiments, AMP-224 is administered (for example by subcutaneous administration) at about 1 to about 50 mg/kg (including for example 5 mg/kg, 10 mg/kg, 15 mg/kg, 20 mg/kg, 25 mg/kg, 30 mg/kg, 35 mg/kg, 40 mg/kg, 45 mg/kg, 50 mg/kg), twice every 28 days. In some embodiments, the other agent is BMS-936559. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), every two weeks. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), once every 4 weeks. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), twice every four weeks. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), three times every six weeks. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), on days 1, 15 and 29 of a 6-week cycle. For example, in some embodiments, BMS-936559, is administered (for example by intravenous infusion) at about 0.1 to about 20 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg), on days 1, 15, and 29 of a 6-week cycle, for up to 16 cycles. In some embodiments, the BMS-936559 is administered as an intravenous infusion over about 10, 20, 30, 40, 50, or 60 or more minutes. In some embodiments, the BMS-936559 is administered as an intravenous infusion over about 60 minutes once every 1, 2, 3, 4, 5 or more weeks. In some embodiments, the BMS-936559 is administered as an intravenous infusion over about 60 minutes once every two weeks. In some embodiments, the BMS-936559 is administered as an intravenous infusion over about 60 minutes twice every three weeks. In some embodiments, the BMS-936559 is administered as an intravenous infusion over about 60 minutes three times every six weeks. For example, in some embodiments, MPDL3280A, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), three times every six weeks. For example, in some embodiments, MPDL3280A, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every three weeks. In some embodiments, the other agent is MPDL3280A. For example, in some embodiments, MPDL3280A, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every four weeks. In some embodiments, the MPDL3280A is administered as an intravenous infusion over about 10, 20, 30, 40, 50, or 60 or more minutes. In some embodiments, the MPDL3280A is administered as an intravenous infusion over about 60 minutes once every 1, 2, 3, 4, 5 or more weeks. In some embodiments, the MPDL3280A is administered as an intravenous infusion over about 60 minutes once every two weeks. In some embodiments, the MPDL3280A is administered as an intravenous infusion over about 60 minutes twice every three weeks. In some embodiments, the MPDL3280A is administered as an intravenous infusion over about 60 minutes once every three weeks. In some embodiments, the other agent is MEDI4736. For example, in some embodiments, MEDI4736, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), three times every six weeks. For example, in some embodiments, MEDI4736, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every three weeks. For example, in some embodiments, MEDI4736, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every four weeks. In some embodiments, the MEDI4736 is administered as an intravenous infusion over about 10, 20, 30, 40, 50, or 60 or more minutes. In some embodiments, the MEDI4736 is administered as an intravenous infusion over about 60 minutes once every 1, 2, 3, 4, 5 or more weeks. In some embodiments, the MEDI4736 is administered as an intravenous infusion over about 60 minutes once every two weeks. In some embodiments, the MEDI4736 is administered as an intravenous infusion over about 60 minutes twice every three weeks. In some embodiments, the MEDI4736 is administered as an intravenous infusion over about 60 minutes once every three weeks. In some embodiments, the other agent is MSB0010718C. For example, in some embodiments, MSB0010718C, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), three times every six weeks. For example, in some embodiments, MSB0010718C, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every three weeks. For example, in some embodiments, MSB0010718C, is administered (for example by intravenous infusion) at about 0.5 to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg), once every four weeks. In some embodiments, the MSB0010718C is administered as an intravenous infusion over about 10, 20, 30, 40, 50, or 60 or more minutes. In some embodiments, the MSB0010718C is administered as an intravenous infusion over about 60 minutes once every 1, 2, 3, 4, 5 or more weeks. In some embodiments, the MSB0010718C is administered as an intravenous infusion over about 60 minutes once every two weeks. In some embodiments, the MSB0010718C is administered as an intravenous infusion over about 60 minutes twice every three weeks. In some embodiments, the MSB0010718C is administered as an intravenous infusion over about 60 minutes once every three weeks.

In some embodiments, the effective amount of taxane in the nanoparticle composition is between about 45 mg/m² to about 350 mg/m² and the effective amount of the other agent is about 0.1 mg/kg to about 20 mg/kg (including for example about 0.1 mg/kg to about 15 mg/kg, about 0.1 mg/kg to about 10 mg/kg, about 0.2 mg/kg to about 10 mg/kg, about 0.3 mg/kg to about 8 mg/kg, about 0.3 mg/kg to about 7 mg/kg, about 0.3 mg/kg to about 6 mg/kg, about 0.3 mg/kg to about 5.5 mg/kg, about 0.3 mg/kg to about 5 mg/kg). In some embodiments, the effective amount of taxane in the nanoparticle composition is between about 80 mg/m² to about 350 mg/m² and the effective amount of the other agent is about 0.1 mg/kg to about 20 mg/kg. In some embodiments, the effective amount of the other agent is at least about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/k, 9 mg/k, or 10 mg/kg.

In some embodiments, the effective amount of taxane in the nanoparticle composition is between about 45 mg/m² to about 350 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 350 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 300 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 150 mg/m² to about 350 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 150 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 100 mg/m². In some embodiments, the effective amount of taxane in the nanoparticle composition is between about 170 mg/m² to about 200 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 200 mg/m² to about 350 mg/m² and the effective amount of the other agent is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 260 mg/m². In some embodiments of any of the above methods, the effective amount of the other agent is about 20-30 mg/kg, about 30-40 mg/kg, about 40-50 mg/kg, about 50-60 mg/kg, about 60-70 mg/kg, about 70-80 mg/kg, about 80-100 mg/kg, or about 100-120 mg/kg.

In some embodiments, the effective amount of taxane in the nanoparticle composition is between about 45 mg/m² to about 350 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 80 mg/m² to about 350 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 80 mg/m² to about 300 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 150 mg/m² to about 350 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 80 mg/m² to about 150 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 170 mg/m² to about 200 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane in the nanoparticle composition is between about 200 mg/m² to about 350 mg/m² and the effective amount of the other agent is about 80 mg to about 1000 mg (including for example about 80 to about 100 mg, about 100 to about 200 mg, about 200 to about 300 mg, about 300 to about 400 mg, about 400 to about 500 mg, about 500 to about 600 mg, about 600 to about 700 mg, about 700 to about 800 mg, about 800 to about 900 mg, about 900 mg to about 1000 mg). In some embodiments, the effective amount of taxane (e.g., paclitaxel) in the nanoparticle composition is about 100 mg/m². In some embodiments of any of the above methods, the effective amount of the other agent is about 100-200 mg, about 200-300 mg, about 300-400 mg, about 400-500 mg.

In some embodiments, the effective amount of paclitaxel in the nanoparticle composition is about 50-300 mg/m² (including for example about 100 mg/m², 125 mg/m², 260 mg/m²) and the effective amount of the other agent (such as nivolumab, pidilizumab) is about 0.1 to about 10 mg/kg (including for example about 0.3 mg/kg, 1 mg/kg, 3 mg/kg, 5 mg/kg). In some embodiments, the effective amount of paclitaxel in the nanoparticle composition is about 50-300 mg/m² (including for example about 100 mg/m², 125 mg/m², 260 mg/m²) and the effective amount of the other agent (such as pembrolizumab, BMS-936559) is about 0.5 to about 20 mg/kg (including for example about 1 mg/kg, 2 mg/kg, 5 mg/kg, 10 mg/kg, 15 mg/kg). In some embodiments, the effective amount of paclitaxel in the nanoparticle composition is about 50-300 mg/m² (including for example about 100 mg/m², 125 mg/m², 260 mg/m²) and the effective amount of the other agent (such as AMP-224) is about 1 to about 50 mg/kg (including for example about 1.5 mg/kg, 5 mg/kg, 15 mg/kg, 45 mg/kg). In some embodiments, the effective amount of paclitaxel in the nanoparticle composition is about 50-300 mg/m² (including for example about 100 mg/m², 125 mg/m², 260 mg/m²) and the effective amount of the other agent (such as MPDL3280A, MEDI4736 and MSB0010718C) is about 0.5 to about 30 mg/kg (including for example about 0.3 mg/kg, 1 mg/kg, 3 mg/kg, 5 mg/kg, 10 mg/kg, 15 mg/kg, 20 mg/kg). In some embodiments, the other agent is MPDL3280A. In some embodiments, the other agent is MEDI4736. In some embodiments, the other agent is MSB0010718C.

The nanoparticle composition (and the other agent) described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In one embodiment, nanoparticles (such as albumin nanoparticles) can be administered by any acceptable route including, but not limited to, orally, intramuscularly, transdermally, intravenously, through an inhaler or other air borne delivery systems and the like.

A combination of the administration configurations described herein can be used. The combination therapy methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of other agents will be approximately those already employed in clinical therapies wherein the other agent are administered alone or in combination with other agents. Variation in dosage will likely occur depending on the condition being treated. As described above, in some embodiments, the other agents may be administered at a reduced level.

### Section V-D. Nanoparticle Compositions

The nanoparticle compositions described herein comprise nanoparticles comprising (in various embodiments consisting essentially of) a taxane (such as paclitaxel) and a carrier protein (such as albumin). Nanoparticles of poorly water soluble drugs (such as taxane) have been disclosed in, for example, U.S. Pat. Nos. 5,916,596; 6,506,405; 6,749,868, 6,537,579 and 7,820,788 and also in U.S. Pat. Pub. Nos. 2006/0263434, and 2007/0082838; PCT Patent Application WO08/137148.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of all the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of all the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the carrier protein has sulfhydral groups that can form disulfide bonds. In some embodiments, at least about 5% (including for example at least about any one of 10%, 15%, 20%, 25%, 30%, 40%, 50%, 60%, 70%, 80%, or 90%) of the carrier protein in the nanoparticle portion of the composition are crosslinked (for example crosslinked through one or more disulfide bonds).

In some embodiments, the nanoparticles comprise the taxane (such as paclitaxel) coated with a carrier protein, such as albumin (e.g., human serum albumin). In some embodiments, the composition comprises taxane in both nanoparticle and non-nanoparticle forms, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane that is substantially free of polymeric materials (such as polymeric matrix).

In some embodiments, the nanoparticle composition is substantially free (such as free) of surfactants (such as CREMOPHOR®, Tween 80, or other organic solvents used for the administration of taxanes). In some embodiments, the nanoparticle composition contains less than about any one of 20%, 15%, 10%, 7.5%, 5%, 2.5%, or 1% organic solvent. In some embodiments, the weight ratio of carrier protein (such as albumin) and taxane in the nanoparticle composition is about 18:1 or less, such as about 15:1 or less, for example about 10:1 or less. In some embodiments, the weight ratio of carrier protein (such as albumin) and taxane in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of carrier protein and taxane in the nanoparticle portion of the composition is about any one of 1:2, 1:3, 1:4, 1:5, 1:10, 1:15, or less. In some embodiments, the weight ratio of the carrier protein (such as albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

The nanoparticles described herein comprise a taxane (such as paclitaxel) and a carrier protein (such as albumin). The term “proteins” refers to polypeptides or polymers of amino acids of any length (including full length or fragments), which may be linear or branched, comprise modified amino acids, and/or be interrupted by non-amino acids. The term also encompasses an amino acid polymer that has been modified naturally or by intervention; for example, disulfide bond formation, glycosylation, lipidation, acetylation, phosphorylation, or any other manipulation or modification. Also included within this term are, for example, polypeptides containing one or more analogs of an amino acid (including, for example, unnatural amino acids, etc.), as well as other modifications known in the art. The proteins described herein may be naturally occurring, i.e., obtained or derived from a natural source (such as blood), or synthesized (such as chemically synthesized or by synthesized by recombinant DNA techniques).

Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the carrier protein is non-blood protein, such as casein, α-lactalbumin, and β-lactoglobulin. The carrier proteins may either be natural in origin or synthetically prepared. In some embodiments, the pharmaceutically acceptable carrier comprises albumin, such as human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The carrier protein (such as albumin) in the composition generally serves as a carrier for the taxane, i.e., the carrier protein in the composition makes the taxane more readily suspendable in an aqueous medium or helps maintain the suspension as compared to compositions not comprising a carrier protein. This can avoid the use of toxic solvents (or surfactants) for solubilizing the taxane, and thereby can reduce one or more side effects of administration of the taxane into an individual (such as a human). Thus, in some embodiments, the composition described herein is substantially free (such as free) of surfactants, such as CREMOPHOR® (including CREMOPHOR EL® (BASF)). In some embodiments, the nanoparticle composition is substantially free (such as free) of surfactants. A composition is “substantially free of CREMOPHOR®” or “substantially free of surfactant” if the amount of CREMOPHOR® or surfactant in the composition is not sufficient to cause one or more side effect(s) in an individual when the nanoparticle composition is administered to the individual.

The amount of carrier protein in the composition described herein will vary depending on other components in the composition. In some embodiments, the composition comprises a carrier protein in an amount that is sufficient to stabilize the taxane in an aqueous suspension, for example, in the form of a stable colloidal suspension (such as a stable suspension of nanoparticles). In some embodiments, the carrier protein is in an amount that reduces the sedimentation rate of the taxane in an aqueous medium. For particle-containing compositions, the amount of the carrier protein also depends on the size and density of nanoparticles of the taxane.

A taxane (such as paclitaxel) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the carrier protein is present in an amount that is sufficient to stabilize the taxane in an aqueous suspension at a certain concentration. For example, the concentration of the taxane in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the carrier protein is present in an amount that avoids use of surfactants (such as CREMOPHOR®), so that the composition is free or substantially free of surfactant (such as CREMOPHOR®).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of carrier protein. In some embodiments, the composition, in liquid form, comprises about 0.5% to about 5% (w/v) of carrier protein.

In some embodiments, the weight ratio of carrier protein, e.g., albumin, to the taxane in the nanoparticle composition is such that a sufficient amount of taxane binds to, or is transported by, the cell. While the weight ratio of carrier protein to taxane will have to be optimized for different carrier protein and taxane combinations, generally the weight ratio of carrier protein, e.g., albumin, to taxane (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the carrier protein to taxane weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the carrier protein (such as albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1.

In some embodiments, the carrier protein allows the composition to be administered to an individual (such as human) without significant side effects. In some embodiments, the carrier protein (such as albumin) is in an amount that is effective to reduce one or more side effects of administration of the taxane to a human. The term “reducing one or more side effects of administration of the taxane” refers to reduction, alleviation, elimination, or avoidance of one or more undesirable effects caused by the taxane, as well as side effects caused by delivery vehicles (such as solvents that render the taxanes suitable for injection) used to deliver the taxane. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxanes can be reduced.

In some embodiments, the composition comprises ABRAXANE® (nab-paclitaxel). ABRAXANE® is a formulation of paclitaxel stabilized by human albumin USP, which can be dispersed in directly injectable physiological solution. When dispersed in a suitable aqueous medium such as 0.9% sodium chloride injection or 5% dextrose injection, ABRAXANE® forms a stable colloidal suspension of paclitaxel. The mean particle size of the nanoparticles in the colloidal suspension is about 130 nanometers. Since HSA is freely soluble in water, ABRAXANE® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle compositions are known in the art. For example, nanoparticles containing taxanes (such as paclitaxel) and carrier protein (such as albumin) can be prepared under conditions of high shear forces (e.g., sonication, high pressure homogenization, or the like). These methods are disclosed in, for example, U.S. Pat. Nos. 5,916,596; 6,506,405; 6,749,868, 6,537,579, 7,820,788 and also in U.S. Pat. Pub. No. 2007/0082838, 2006/0263434 and PCT Application WO08/137148.

Briefly, the taxane (such as paclitaxel) is dissolved in an organic solvent, and the solution can be added to a human serum albumin solution. The mixture is subjected to high pressure homogenization. The organic solvent can then be removed by evaporation. The dispersion obtained can be further lyophilized. Suitable organic solvents include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### V-D1. Other Components in the Nanoparticle Compositions

The nanoparticles described herein can be present in a composition that includes other agents, excipients, or stabilizers. For example, to increase stability by increasing the negative zeta potential of nanoparticles, certain negatively charged components may be added. Such negatively charged components include, but are not limited to bile salts of bile acids consisting of glycocholic acid, cholic acid, chenodeoxycholic acid, taurocholic acid, glycochenodeoxycholic acid, taurochenodeoxycholic acid, litocholic acid, ursodeoxycholic acid, dehydrocholic acid and others; phospholipids including lecithin (egg yolk) based phospholipids which include the following phosphatidylcholines: palmitoyloleoylphosphatidylcholine, palmitoyllinoleoylphosphatidylcholine, stearoyllinoleoylphosphatidylcholine stearoyloleoylphosphatidylcholine, stearoylarachidoylphosphatidylcholine, and dipalmitoylphosphatidylcholine. Other phospholipids including L-α-dimyristoylphosphatidylcholine (DMPC), dioleoylphosphatidylcholine (DOPC), distearyolphosphatidylcholine (DSPC), hydrogenated soy phosphatidylcholine (HSPC), and other related compounds. Negatively charged surfactants or emulsifiers are also suitable as additives, e.g., sodium cholesteryl sulfate and the like.

In some embodiments, the composition is suitable for administration to a human. In some embodiments, the composition is suitable for administration to a mammal such as, in the veterinary context, domestic pets and agricultural animals. There are a wide variety of suitable formulations of the nanoparticle composition (see, e.g., U.S. Pat. Nos. 5,916,596, 6,096,331 and 7,820,788). The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of any of about 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### Section V-E. Kits and Compositions

Also provided herein are compositions (such as pharmaceutical compositions), kits, and unit dosages useful for methods described herein. Also provided are any use described herein whether in the context of use as a medicament and/or use for manufacture of a medicament.

Kits described herein include one or more containers comprising a taxane-containing nanoparticle compositions (or unit dosage forms and/or articles of manufacture) and/or at least one other agent that antagonizes a PD-1 pathway in a cell, and in some embodiments, further comprise instructions for use in accordance with any of the methods described herein. The kit may further comprise a description of selection an individual suitable or treatment. Instructions supplied in the kits described herein are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising nanoparticles comprising a taxane (such as paclitaxel) and a carrier protein (such as albumin), and b) an effective amount of at least one other agent that antagonizes a PD-1 pathway in a cell. In some embodiments, the kit comprises a) a composition comprising nanoparticles comprising a taxane (such as paclitaxel) and a carrier protein (such as albumin), b) an effective amount of at least one other agent that antagonizes a PD-1 pathway in a cell, and c) instructions for administering the nanoparticles and the other agents simultaneously, sequentially, or concurrently for treatment of a proliferative disease (such as cancer). In some embodiments, the taxane is any of paclitaxel, tesetaxel, docetaxel, and ortataxel. In some embodiments, the kit comprises nanoparticles comprising a) a composition comprising nanoparticles comprising paclitaxel coated with an albumin (such as nab-paclitaxel, e.g., ABRAXANE®), b) an effective amount of at least one other agent that antagonizes a PD-1 pathway in a cell, and c) instructions for administering the nanoparticles and the other agents simultaneously, sequentially, and/or concurrently, for the effective treatment of a proliferative disease (such as cancer). In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011) and rHIgM12B7. In some embodiments, the other agent is selected from the group consisting of AMP-514, pembrolizumab (MK-3475), REGN2810, PDR001, BGB-A317, and pidilizumab (CT-011). In some embodiments, the other agent is selected from the group consisting of BMS-936559, MEDI4736, MSB0010718C and MPDL3280A. In some embodiments, the other agent is rHIgM12B7. In some embodiments, the other agent is a PD-1 antagonist. In some embodiments, the other agent is a PD-L1 antagonist. In some embodiments, the other agent is a PD-L2 antagonist. In some embodiments, the other agent is an antibody. In some embodiments, the other agent is an anti-PD-1 antibody. In some embodiments, the other agent is an anti-PD-L1 antibody. In some embodiments, the other agent is an anti-PD-L2 antibody. In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is rHIgM12B7. In some embodiments, the kit further comprises one or more chemotherapeutic agents. In some embodiments, the one or more chemotherapeutic agents comprises a platinum-based agent. In some embodiments, the platinum-based agent is carboplatin. In some embodiments, the platinum-based agent is cisplatin. In some embodiments, the one or more chemotherapeutic agents comprises a nucleoside analog. In some embodiments, the nucleoside analog comprises gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, the proliferative disease is cancer. In some embodiments, the cancer is breast cancer. In some embodiments, the breast cancer is a Her2 negative breast cancer, metastatic breast cancer, recurrent breast, or a combination thereof. In some embodiments, the cancer is lung cancer. In some embodiments, the lung cancer is NSCLC. In some embodiments, the cancer is pancreatic cancer. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

In some embodiments, the kit comprises a) a composition comprising nanoparticles comprising a taxane (such as paclitaxel) and a carrier protein (such as albumin), and b) an effective amount of at least one other agent that antagonizes PD-1. In some embodiments, the kit comprises a) a composition comprising nanoparticles comprising a taxane (such as paclitaxel) and a carrier protein (such as albumin), b) an effective amount of at least one other agent that antagonizes a PD-1, and c) instructions for administering the nanoparticles and the other agents simultaneously, sequentially, or concurrently for treatment of a proliferative disease (such as cancer). In some embodiments, the taxane is any of paclitaxel, tesetaxel, docetaxel, and ortataxel. In some embodiments, the kit comprises nanoparticles comprising a) a composition comprising nanoparticles comprising paclitaxel coated with an albumin (such as nab-paclitaxel, e.g., ABRAXANE®), b) an effective amount of at least one other agent that antagonizes a PD-1, and c) instructions for administering the nanoparticles and the other agents simultaneously, sequentially, and/or concurrently, for the effective treatment of a proliferative disease (such as cancer). In some embodiments, the other agent is selected from the group consisting of AMP-224, nivolumab, AMP-514, pembrolizumab (MK-3475), REGN2810, PDR001, BGB-A317, and pidilizumab (CT-011). In some embodiments, the other agent is an antibody. In some embodiments, the other agent is an anti-PD-1 antibody. In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, the kit further comprises one or more chemotherapeutic agents. In some embodiments, the one or more chemotherapeutic agents comprises a platinum-based agent. In some embodiments, the platinum-based agent is carboplatin. In some embodiments, the one or more chemotherapeutic agents comprises a nucleoside analog. In some embodiments, the nucleoside analog comprises gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, the proliferative disease is cancer. In some embodiments, the cancer is breast cancer. In some embodiments, the breast cancer is a Her2 negative breast cancer, metastatic breast cancer, recurrent breast, or a combination thereof. In some embodiments, the cancer is lung cancer. In some embodiments, the lung cancer is NSCLC. In some embodiments, the cancer is pancreatic cancer. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

In some embodiments, the kit comprises a) a composition comprising nanoparticles comprising a taxane (such as paclitaxel) and a carrier protein (such as albumin), and b) an effective amount of at least one other agent that antagonizes a ligand of PD-1. In some embodiments, the kit comprises a) a composition comprising nanoparticles comprising a taxane (such as paclitaxel) and a carrier protein (such as albumin), b) an effective amount of at least one other agent that antagonizes a ligand of PD-1, and c) instructions for administering the nanoparticles and the other agents simultaneously, sequentially, or concurrently for treatment of a proliferative disease (such as cancer). In some embodiments, the taxane is any of paclitaxel, tesetaxel, docetaxel, and ortataxel. In some embodiments, the kit comprises nanoparticles comprising a) a composition comprising nanoparticles comprising paclitaxel coated with an albumin (such as nab-paclitaxel, e.g., ABRAXANE®), b) an effective amount of at least one other agent that antagonizes a ligand of PD-1, and c) instructions for administering the nanoparticles and the other agents simultaneously, sequentially, and/or concurrently, for the effective treatment of a proliferative disease (such as cancer). In some embodiments, the other agent is selected from the group consisting of BMS-936559, MEDI4736, MSB0010718C and MPDL3280A. In some embodiments, the other agent is BMS-936559. In some embodiments, the other agent is MEDI4736. In some embodiments, the other agent is MSB0010718C. In some embodiments, the other agent is MPDL3280A (RG7446). In some embodiments, the other agent is a PD-L1 antagonist. In some embodiments, the other agent is a PD-L2 antagonist. In some embodiments, the other agent is an antibody. In some embodiments, the other agent is an anti-PD-L1 antibody. In some embodiments, the other agent is an anti-PD-L2 antibody, e.g., rHIgM12B7. In some embodiments, the kit further comprises one or more chemotherapeutic agents. In some embodiments, the one or more chemotherapeutic agents comprises a platinum-based agent. In some embodiments, the platinum-based agent is carboplatin. In some embodiments, the one or more chemotherapeutic agents comprises a nucleoside analog. In some embodiments, the nucleoside analog comprises gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, the proliferative disease is cancer. In some embodiments, the cancer is breast cancer. In some embodiments, the breast cancer is a Her2 negative breast cancer, metastatic breast cancer, recurrent breast, or a combination thereof. In some embodiments, the cancer is lung cancer. In some embodiments, the lung cancer is NSCLC. In some embodiments, the cancer is pancreatic cancer. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

In some embodiments, a kit comprises a) a composition comprising nanoparticles comprising a taxane (such as paclitaxel) coated with a carrier protein (such as albumin), b) a composition comprising nanoparticles comprising at least one other agent that antagonizes a PD-1 pathway in a cell and a carrier protein (such as albumin), and c) instructions for administering the nanoparticle compositions simultaneously, sequentially, and/or concurrently, for treatment of a proliferative disease (such as cancer). In some embodiments, the kit comprises nanoparticles comprising a) a composition comprising nanoparticles comprising paclitaxel coated with an albumin (such as nab-paclitaxel, e.g., ABRAXANE®), b) a composition comprising nanoparticles comprising at least one other agent that antagonizes a PD-1 pathway in a cell and a carrier protein (such as albumin), and c) instructions for administering the nanoparticle compositions simultaneously, sequentially, and/or concurrently, for the effective treatment of a proliferative disease (such as cancer). In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011) and rHIgM12B7. In some embodiments, the other agent is selected from the group consisting of AMP-514, pembrolizumab (MK-3475), REGN2810, PDR001, BGB-A317, and pidilizumab (CT-011). In some embodiments, the other agent is selected from the group consisting of BMS-936559, MEDI4736, MSB0010718C and MPDL3280A. In some embodiments, the other agent is a PD-1 antagonist. In some embodiments, the other agent is a PD-L1 antagonist. In some embodiments, the other agent is a PD-L2 antagonist, e.g., rHIgM12B7. In some embodiments, the other agent is an antibody. In some embodiments, the other agent is an anti-PD-1 antibody. In some embodiments, the other agent is an anti-PD-L1 antibody. In some embodiments, the other agent is an anti-PD-L2 antibody. In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011). In some embodiments, a PD-L1 antibody is BMS-936559. In some embodiments, a PD-L1 antibody is MEDI4736. In some embodiments, a PD-L1 antibody is MPDL3280A. In some embodiments, a PD-L1 antibody is MSB0010718C. In some embodiments, the kit further comprises one or more chemotherapeutic agents. In some embodiments, the one or more chemotherapeutic agents comprises a platinum-based agent. In some embodiments, the platinum-based agent is carboplatin. In some embodiments, the one or more chemotherapeutic agents comprises a nucleoside analog. In some embodiments, the nucleoside analog comprises gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle. In some embodiments, the proliferative disease is cancer. In some embodiments, the cancer is breast cancer. In some embodiments, the breast cancer is a Her2 negative breast cancer, metastatic breast cancer, recurrent breast, or a combination thereof. In some embodiments, the cancer is lung cancer. In some embodiments, the lung cancer is NSCLC. In some embodiments, the cancer is pancreatic cancer. In some embodiments, the pancreatic cancer is advanced metastatic pancreatic cancer. In some embodiments, the pancreatic cancer is locally advanced, surgically resected or unresected pancreatic cancer or metastatic pancreatic adenocarcinoma.

The nanoparticles and the other agents can be present in separate containers or in a single container. It is understood that the kit may comprise one distinct composition or two or more compositions wherein one composition comprises nanoparticles and one composition comprises an other agent.

The kits provided herein are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the nanoparticle compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the taxane (such as taxane) as disclosed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the taxane and pharmaceutical compositions and instructions for use and packaged in quantities sufficient for storage and use in pharmacies, for example, hospital pharmacies and compounding pharmacies.

Also provided are pharmaceutical compositions for treating proliferative diseases. In some embodiments, the pharmaceutical composition comprises a) a composition comprising nanoparticles comprising a taxane (such as paclitaxel) and a carrier protein (such as albumin), and b) at least one other agent that antagonizes a PD-1 pathway in a cell. In some embodiments, the taxane is any of paclitaxel, tesetaxel, docetaxel, and ortataxel. In some embodiments, the pharmaceutical composition comprises nanoparticles comprising a) a composition comprising nanoparticles comprising paclitaxel coated with an albumin (such as nab-paclitaxel, e.g., ABRAXANE®), and b) at least one other agent that antagonizes a PD-1 pathway in a cell. In some embodiments, the pharmaceutical composition is used for the effective treatment of a proliferative disease (such as cancer). In some embodiments, instructions for administering the composition comprising nanoparticles and the other agent are provided. In some embodiments, the instructions provide for administering the composition comprising nanoparticles and the other agents simultaneously, sequentially, and/or concurrently. In some embodiments, the other agent is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, pidilizumab (CT-011), and rHIgM12B7. In some embodiments, the other agent is a PD-1 antagonist. In some embodiments, the other agent is an antibody. In some embodiments, the other agent is an anti-PD-1 antibody. In some embodiments, the other agent is nivolumab. In some embodiments, the other agent is AMP-514. In some embodiments, the other agent is pembrolizumab (MK-3475). In some embodiments, the other agent is REGN2810. In some embodiments, the other agent is PDR001. In some embodiments, the other agent is BGB-A317. In some embodiments, the other agent is pidilizumab (CT-011).

Also provided are pharmaceutical compositions for treating a lung cancer (for example, NSCLC). In some embodiments, the pharmaceutical composition comprises a) a composition comprising nanoparticles comprising a taxane (such as paclitaxel) and a carrier protein (such as albumin), and b) at least one other agent that antagonizes a PD-1 pathway in a cell. In some embodiments, the taxane is any of paclitaxel, tesetaxel, docetaxel, and ortataxel. In some embodiments there is provided a pharmaceutical composition comprising a) a composition comprising nanoparticles comprising paclitaxel coated with an albumin (such as nab-paclitaxel, e.g., ABRAXANE®), and b) at least one other agent that antagonizes a PD-1 pathway in a cell. In some embodiments, the pharmaceutical composition is used for the effective treatment of a lung cancer (such as NSCLC). In some embodiments, instructions for administering the composition comprising nanoparticles and the other agent are provided. In some embodiments, the instructions provide for administering the composition comprising nanoparticles and the other agents simultaneously, sequentially, and/or concurrently. In some embodiments, the at least one other agent that antagonizes a PD-1 pathway in a cell comprises an antibody. In some embodiments, the at least one other agent that antagonizes a PD-1 pathway in a cell is an anti-PD-1 antibody. In some embodiments, the at least one other agent that antagonizes a PD-1 pathway in a cell is an anti-PD-L1 antibody. In some embodiments, the at least one other agent that antagonizes a PD-1 pathway in a cell is an anti-PD-L2 antibody. In some embodiments, the at least one other agent that antagonizes a PD-1 pathway in a cell comprises a fusion peptide. In some embodiments, the at least one other agent that antagonizes a PD-1 pathway in a cell is a PD-1 antagonist. In some embodiments, the at least one other agent that antagonizes a PD-1 pathway in a cell is selected from the group consisting of nivolumab, AMP-514, pembrolizumab (MK-3475), REGN2810, PDR001, BGB-A317, pidilizumab (CT-011), and rHIgM12B7. In some embodiments, the at least one other agent that antagonizes a PD-1 pathway in a cell is nivolumab. In some embodiments, the at least one other agent that antagonizes a PD-1 pathway in a cell is AMP-514. In some embodiments, the at least one other agent that antagonizes a PD-1 pathway in a cell is pembrolizumab (MK-3475). In some embodiments, the at least one other agent that antagonizes a PD-1 pathway in a cell is REGN2810. In some embodiments, the at least one other agent that antagonizes a PD-1 pathway in a cell is PDR001. In some embodiments, the at least one other agent that antagonizes a PD-1 pathway in a cell is BGB-A317. In some embodiments, the at least one other agent that antagonizes a PD-1 pathway in a cell is pidilizumab (CT-011). In some embodiments, the at least one other agent that antagonizes a PD-1 pathway in a cell is an antagonist of a PD-1 ligand. In some embodiments, the at least one other agent that antagonizes a PD-1 pathway in a cell is a PD-L1 antagonist. In some embodiments, the at least one other agent that antagonizes a PD-1 pathway in a cell is a PD-L2 antagonist. In some embodiments, the at least one other agent that antagonizes a PD-1 pathway in a cell is selected from the group consisting of BMS-936559, MEDI4736, MSB0010718C and MPDL3280A (RG7446). In some embodiments, the at least one other agent that antagonizes a PD-1 pathway is BMS-936559. In some embodiments, the at least one other agent that antagonizes a PD-1 pathway is MEDI4736. In some embodiments, the at least one other agent that antagonizes a PD-1 pathway is MPDL3280A. In some embodiments, the at least one other agent that antagonizes a PD-1 pathway is MSB0010718C. In some embodiments, the at least one other agent that antagonizes a PD-1 pathway in a cell is AMP-224. In some embodiments there is provided a pharmaceutical composition comprising a) a composition comprising nanoparticles comprising paclitaxel coated with an albumin (such as nab-paclitaxel, e.g., ABRAXANE®), and b) a PD-1 antagonist (such as nivolumab, AMP-514, pembrolizumab (MK-3475), REGN2810, PDR001, BGB-A317, and pidilizumab (CT-011)). In some embodiments, the PD-1 antagonist is nivolumab. In some embodiments, the PD-1 antagonist is AMP-514. In some embodiments, the PD-1 antagonist is pembrolizumab (MK-3475). In some embodiments, the PD-1 antagonist is REGN2810. In some embodiments, the PD-1 antagonist is PDR001. In some embodiments, the PD-1 antagonist is BGB-A317. In some embodiments, the PD-1 antagonist is pidilizumab (CT-011). In some embodiments, the pharmaceutical composition is used for the effective treatment of lung cancer (such as NSCLC). In some embodiments, instructions for administering the composition comprising nanoparticles and the other agent are provided. In some embodiments, the instructions provide for administering the composition comprising nanoparticles and the PD-1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) nivolumab. In some embodiments, the pharmaceutical composition is used for the effective treatment of lung cancer (such as NSCLC). In some embodiments, instructions for administering the composition comprising nanoparticles and the nivolumab are provided. In some embodiments, the instructions provide for administering the composition comprising nanoparticles and the nivolumab simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L1 antagonist (such as BMS-936559, MEDI4736, MSB0010718C or MPDL3280A (RG7446)). In some embodiments, the PD-L1 antagonist is BMS-936559. In some embodiments, the PD-L1 antagonist is MEDI4736. In some embodiments, the PD-L1 antagonist is MSB0010718C. In some embodiments, the PD-L1 antagonist is MPDL3280A (RG7446). In some embodiments, the pharmaceutical composition is used for the effective treatment of lung cancer (such as NSCLC). In some embodiments, instructions for administering the composition comprising nanoparticles and the other agent are provided. In some embodiments, the instructions provide for administering the composition comprising nanoparticles and the PD-L1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L2 antagonist (such as an anti-PD-L2 antibody, for example, rHIgM12B7). In some embodiments, the pharmaceutical composition is used for the effective treatment of lung cancer (such as NSCLC). In some embodiments, instructions for administering the composition comprising nanoparticles and the PD-L2 antagonist are provided. In some embodiments, the instructions provide for administering the composition comprising nanoparticles and the PD-L2 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) AMP-224. In some embodiments, the pharmaceutical composition is used for the effective treatment of lung cancer (such as NSCLC). In some embodiments, instructions for administering the composition comprising nanoparticles and the AMP-224 are provided. In some embodiments, the instructions provide for administering the composition comprising nanoparticles and the AMP-224 simultaneously, sequentially, and/or concurrently. In some embodiments, the kit further comprises one or more chemotherapeutic agents. In some embodiments, the one or more chemotherapeutic agents comprises a platinum-based agent. In some embodiments, the platinum-based agent is carboplatin.

Also provided are pharmaceutical compositions for treating breast cancer (such as Her2 negative breast cancer, metastatic breast cancer). In some embodiments, the pharmaceutical composition comprises a) a composition comprising nanoparticles comprising a taxane (such as paclitaxel) and a carrier protein (such as albumin), and b) at least one other agent that antagonizes a PD-1 pathway in a cell. In some embodiments, the taxane is any of paclitaxel, tesetaxel, docetaxel, and ortataxel. In some embodiments there is provided a pharmaceutical composition comprising a) a composition comprising nanoparticles comprising paclitaxel coated with an albumin (such as nab-paclitaxel, e.g., ABRAXANE®), and b) at least one other agent that antagonizes a PD-1 pathway in a cell. In some embodiments, the pharmaceutical composition is used for the effective treatment of breast cancer (such as Her2 negative breast cancer, metastatic breast cancer). In some embodiments, instructions for administering the composition comprising nanoparticles and the other agent are provided. In some embodiments, the instructions provide for administering the composition comprising nanoparticles and the other agent simultaneously, sequentially, and/or concurrently. In some embodiments, the at least one other agent that antagonizes a PD-1 pathway in a cell comprises an antibody. In some embodiments, the at least one other agent that antagonizes a PD-1 pathway in a cell an anti-PD-1 antibody. In some embodiments, the at least one other agent that antagonizes a PD-1 pathway in a cell an anti-PD-L1 antibody. In some embodiments, the at least one other agent that antagonizes a PD-1 pathway in a cell an anti-PD-L2 antibody. In some embodiments, the at least one other agent that antagonizes a PD-1 pathway in a cell comprises a fusion peptide. In some embodiments, the at least one other agent that antagonizes a PD-1 pathway in a cell is a PD-1 antagonist. In some embodiments, the at least one other agent that antagonizes a PD-1 pathway in a cell is selected from the group consisting of nivolumab, AMP-514, pembrolizumab (MK-3475), REGN2810, PDR001, BGB-A317, pidilizumab (CT-011), and rHIgM12B7. In some embodiments, the at least one other agent that antagonizes a PD-1 pathway in a cell is nivolumab. In some embodiments, the at least one other agent that antagonizes a PD-1 pathway in a cell is AMP-514. In some embodiments, the at least one other agent that antagonizes a PD-1 pathway in a cell is pembrolizumab (MK-3475). In some embodiments, the at least one other agent that antagonizes a PD-1 pathway in a cell is REGN2810. In some embodiments, the at least one other agent that antagonizes a PD-1 pathway in a cell is PDR001. In some embodiments, the at least one other agent that antagonizes a PD-1 pathway in a cell is BGB-A317. In some embodiments, the at least one other agent that antagonizes a PD-1 pathway in a cell is pidilizumab (CT-011). In some embodiments, the at least one other agent that antagonizes a PD-1 pathway in a cell is an antagonist of a PD-1 ligand. In some embodiments, the at least one other agent that antagonizes a PD-1 pathway in a cell is a PD-L1 antagonist. In some embodiments, the at least one other agent that antagonizes a PD-1 pathway in a cell is a PD-L2 antagonist, for example, an anti-PD-L2 antibody, e.g., rHIgM12B7. In some embodiments, the at least one other agent that antagonizes a PD-1 pathway in a cell is selected from the group consisting of BMS-936559, MEDI4736, MSB0010718C and MPDL3280A (RG7446). In some embodiments, the at least one agent that antagonizes a PD-1 pathway is BMS-936559. In some embodiments, the at least one other agent that antagonizes a PD-1 pathway is MEDI4736. In some embodiments, the at least one other agent that antagonizes a PD-1 pathway is MPDL3280A. In some embodiments, the at least one agent that antagonizes a PD-1 pathway is MSB0010718C. In some embodiments, the at least one other agent that antagonizes a PD-1 pathway in a cell is AMP-224. In some embodiments there is provided a pharmaceutical composition comprising a) a composition comprising nanoparticles comprising paclitaxel coated with an albumin (such as nab-paclitaxel, e.g., ABRAXANE®), and b) a PD-1 antagonist (such as nivolumab, AMP-514, pembrolizumab (MK-3475), REGN2810, PDR001, BGB-A317, pidilizumab (CT-011), and rHIgM12B7. In some embodiments, the PD-1 antagonist is nivolumab. In some embodiments, the PD-1 antagonist is AMP-514. In some embodiments, the PD-1 antagonist is pembrolizumab (MK-3475). In some embodiments, the PD-1 antagonist is REGN2810. In some embodiments, the PD-1 antagonist is PDR001. In some embodiments, the PD-1 antagonist is BGB-A317. In some embodiments, the PD-1 antagonist is pidilizumab (CT-011). In some embodiments, the pharmaceutical composition is used for the effective treatment of breast cancer (such as Her2 negative breast cancer, metastatic breast cancer). In some embodiments, instructions for administering the composition comprising nanoparticles and the PD-1 antagonist are provided. In some embodiments, the instructions provide for administering the composition comprising nanoparticles and the PD-1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) nivolumab. In some embodiments, the pharmaceutical composition is used for the effective treatment of breast cancer (such as Her2 negative breast cancer, metastatic breast cancer). In some embodiments, instructions for administering the composition comprising nanoparticles and the nivolumab are provided. In some embodiments, the instructions provide for administering the composition comprising nanoparticles and the nivolumab simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L1 antagonist (such as BMS-936559, MEDI4736, MSB0010718C or MPDL3280A (RG7446)). In some embodiments, the PD-L1 antagonist is BMS-936559. In some embodiments, the PD-L1 antagonist is MEDI4736. In some embodiments, the PD-L1 antagonist is MSB0010718C. In some embodiments, the PD-L1 antagonist is MPDL3280A (RG7446). In some embodiments, the pharmaceutical composition is used for the effective treatment of breast cancer (such as Her2 negative breast cancer, metastatic breast cancer). In some embodiments, instructions for administering the composition comprising nanoparticles and the PD-L1 antagonist are provided. In some embodiments, the instructions provide for administering the composition comprising nanoparticles and the PD-L1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L2 antagonist (such as an anti-PD-L2 antibody, for example, rHIgM12B7). In some embodiments, the pharmaceutical composition is used for the effective treatment of breast cancer (such as Her2 negative breast cancer, metastatic breast cancer). In some embodiments, instructions for administering the composition comprising nanoparticles and the PD-L2 antagonist are provided. In some embodiments, the instructions provide for administering the composition comprising nanoparticles and the PD-L2 antagonist simultaneously, sequentially, and/or concurrently. In some embodiments there is provided a pharmaceutical comprising a) a composition comprising nanoparticles comprising paclitaxel coated with an albumin (such as nab-paclitaxel, e.g., ABRAXANE®), and b) AMP-224. In some embodiments, the pharmaceutical composition is used for the effective treatment of breast cancer (such as Her2 negative breast cancer, metastatic breast cancer). In some embodiments, instructions for administering the composition comprising nanoparticles and the other agent are provided. In some embodiments, the instructions provide for administering the composition comprising nanoparticles and the AMP-224 simultaneously, sequentially, and/or concurrently.

Also provided are pharmaceutical compositions for treating pancreatic cancer. In some embodiments, the pharmaceutical composition comprises a) a composition comprising nanoparticles comprising a taxane (such as paclitaxel) and a carrier protein (such as albumin), and b) at least one other agent that antagonizes a PD-1 pathway in a cell. In some embodiments, the taxane is any of paclitaxel, tesetaxel, docetaxel, and ortataxel. In some embodiments there is provided a pharmaceutical composition comprising a) a composition comprising nanoparticles comprising paclitaxel coated with an albumin (such as nab-paclitaxel, e.g., ABRAXANE®), and b) at least one other agent that antagonizes a PD-1 pathway in a cell. In some embodiments, the pharmaceutical composition is used for the effective treatment of pancreatic cancer. In some embodiments, instructions for administering the composition comprising nanoparticles and the other agent are provided. In some embodiments, the instructions provide for administering the composition comprising nanoparticles and the other agent simultaneously, sequentially, and/or concurrently. In some embodiments, the at least one other agent that antagonizes a PD-1 pathway in a cell comprises an antibody. In some embodiments, the at least one other agent that antagonizes a PD-1 pathway in a cell an anti-PD-1 antibody. In some embodiments, the at least one other agent that antagonizes a PD-1 pathway in a cell an anti-PD-L1 antibody. In some embodiments, the at least one other agent that antagonizes a PD-1 pathway in a cell an anti-PD-L2 antibody. In some embodiments, the at least one other agent that antagonizes a PD-1 pathway in a cell comprises a fusion peptide. In some embodiments, the at least one other agent that antagonizes a PD-1 pathway in a cell is a PD-1 antagonist. In some embodiments, the at least one other agent that antagonizes a PD-1 pathway in a cell is selected from the group consisting of nivolumab, AMP-514, pembrolizumab (MK-3475), REGN2810, PDR001, BGB-A317, and pidilizumab (CT-011). In some embodiments, the at least one other agent that antagonizes a PD-1 pathway in a cell is nivolumab. In some embodiments, the at least one other agent that antagonizes a PD-1 pathway in a cell is AMP-514. In some embodiments, the at least one other agent that antagonizes a PD-1 pathway in a cell is pembrolizumab (MK-3475). In some embodiments, the at least one other agent that antagonizes a PD-1 pathway in a cell is REGN2810. In some embodiments, the at least one other agent that antagonizes a PD-1 pathway in a cell is PDR001. In some embodiments, the at least one other agent that antagonizes a PD-1 pathway in a cell is BGB-A317. In some embodiments, the at least one other agent that antagonizes a PD-1 pathway in a cell is pidilizumab (CT-011). In some embodiments, the at least one other agent that antagonizes a PD-1 pathway in a cell is an antagonist of a PD-1 ligand. In some embodiments, the at least one other agent that antagonizes a PD-1 pathway in a cell is a PD-L1 antagonist. In some embodiments, the at least one other agent that antagonizes a PD-1 pathway in a cell is a PD-L2 antagonist, for example, an anti-PD-L2 antibody, e.g., rHIgM12B7. In some embodiments, the at least one other agent that antagonizes a PD-1 pathway in a cell is selected from the group consisting of BMS-936559, MEDI4736, MSB0010718C and MPDL3280A (RG7446). In some embodiments, the at least one agent that antagonizes a PD-1 pathway in a cell is BMS-936559. In some embodiments, the at least one other agent that antagonizes a PD-1 pathway in a cell is MEDI4736. In some embodiments, the at least one other agent that antagonizes a PD-1 pathway in a cell is MPDL3280A. In some embodiments, the at least one other agent that antagonizes a PD-1 pathway in a cell is MSB0010718C. In some embodiments, the at least one other agent that antagonizes a PD-1 pathway in a cell is AMP-224. In some embodiments there is provided a pharmaceutical composition comprising a) a composition comprising nanoparticles comprising paclitaxel coated with an albumin (such as nab-paclitaxel, e.g., ABRAXANE®), and b) a PD-1 antagonist (such as nivolumab, AMP-514, pembrolizumab (MK-3475), REGN2810, PDR001, BGB-A317, and pidilizumab (CT-011)). In some embodiments, the PD-1 antagonist is nivolumab. In some embodiments, the PD-1 antagonist is AMP-514. In some embodiments, the PD-1 antagonist is pembrolizumab (MK-3475). In some embodiments, the PD-1 antagonist is REGN2810. In some embodiments, the PD-1 antagonist is PDR001. In some embodiments, the PD-1 antagonist is BGB-A317. In some embodiments, the PD-1 antagonist is pidilizumab (CT-011). In some embodiments, the pharmaceutical composition is used for the effective treatment of pancreatic cancer. In some embodiments, instructions for administering the composition comprising nanoparticles and the PD-1 antagonist are provided. In some embodiments, the instructions provide for administering the composition comprising nanoparticles and the PD-1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) nivolumab. In some embodiments, the pharmaceutical composition is used for the effective treatment of pancreatic cancer. In some embodiments, instructions for administering the composition comprising nanoparticles and the nivolumab are provided. In some embodiments, the instructions provide for administering the composition comprising nanoparticles and the nivolumab simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L1 antagonist (such as BMS-936559, MEDI4736, MSB0010718C or MPDL3280A (RG7446)). In some embodiments, the PD-L1 antagonist is BMS-936559. In some embodiments, the PD-L1 antagonist is MEDI4736. In some embodiments, the PD-L1 antagonist is MSB0010718C. In some embodiments, the PD-L1 antagonist is MPDL3280A (RG7446). In some embodiments, the pharmaceutical composition is used for the effective treatment of pancreatic cancer. In some embodiments, instructions for administering the composition comprising nanoparticles and the PD-L1 antagonist are provided. In some embodiments, the instructions provide for administering the composition comprising nanoparticles and the PD-L1 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) a PD-L2 antagonist (such as an anti-PD-L2 antibody, for example, rHIgM12B7). In some embodiments, the pharmaceutical composition is used for the effective treatment of pancreatic cancer. In some embodiments, instructions for administering the composition comprising nanoparticles and the PD-L2 antagonist are provided. In some embodiments, the instructions provide for administering the composition comprising nanoparticles and the PD-L2 antagonist simultaneously, sequentially, and/or concurrently. In some embodiments there is provided a pharmaceutical composition comprising a) a composition comprising nanoparticles comprising paclitaxel coated with an albumin (such as nab-paclitaxel, e.g., ABRAXANE®), and b) AMP-224, and c) instructions for administering the nanoparticles and the AMP-224 simultaneously, sequentially, and/or concurrently, for the effective treatment of pancreatic cancer. In some embodiments, the pharmaceutical composition is used for the effective treatment of pancreatic cancer. In some embodiments, instructions for administering the composition comprising nanoparticles and the AMP-224 are provided. In some embodiments, the instructions provide for administering the composition comprising nanoparticles and the AMP-224 simultaneously, sequentially, and/or concurrently. In some embodiments, the pharmaceutical composition further comprises an additional chemotherapeutic agent. In some embodiments, the chemotherapeutic agent is a nucleoside analog. In some embodiments, the nucleoside analog is gemcitabine.

The nanoparticles and the other agents can be present in separate containers or in a single container. It is understood that the medicine may comprise one distinct composition or two or more compositions wherein one composition comprises nanoparticles and one composition comprises another agent.

The kits, medicines, and compositions presented herein may include any one or more aspects or parameters described herein.

### Section V-F. Exemplary Embodiments

In one aspect, provided herein is a method of treating a proliferative disease in an individual comprising administering to the individual: a) an effective amount of a composition comprising nanoparticles comprising a taxane (e.g., paclitaxel) and a carrier protein (e.g., albumin), and b) an effective amount of at least one other agent that antagonizes a PD-1 pathway in a cell.

In an embodiment of the above aspect, said other agent is a PD-1 pathway antagonist. In some embodiments, the PD-1 pathway antagonist is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, and pidilizumab (CT-0111).

In an embodiment of the above aspect, said other agent is an anti-PD-1 antibody. In some embodiments, the anti-PD-1 antibody is selected from the group consisting of nivolumab, AMP-514, pembrolizumab (MK-3475), REGN2810, PDR001, BGB-A317, and pidilizumab (CT-0111).

In an embodiment of any of the above embodiments, said other agent is nivolumab. In some embodiments, between about 0.01 mg/kg to about 10 mg/kg of nivolumab is administered. In some embodiments, between about 1 mg/kg to about 10 mg/kg of nivolumab is administered. In some embodiments, between about 3 mg/kg to about 10 mg/kg of nivolumab is administered. In some embodiments, about 10 mg/kg of nivolumab is administered. In some embodiments, at least about 7 mg/kg of nivolumab is administered. In some embodiments, at least about 5 mg/kg of nivolumab is administered. In some embodiments, at least about 3 mg/kg of nivolumab is administered. In some embodiments, at least about 1 mg/kg of nivolumab is administered. In some embodiments, at least about 0.3 mg/kg of nivolumab is administered. In some embodiments, the nivolumab is administered at least once during a cycle. In some embodiments, the nivolumab is administered at least twice during a cycle. In some embodiments, the nivolumab is administered at least once a week. In some embodiments, the nivolumab is administered at least once every two weeks. In some embodiments, the nivolumab is administered at least once every three weeks. In some embodiments, the nivolumab is administered at least once every four weeks. In some embodiments, a cycle is 21 days. In some embodiments, a cycle is 28 days. In some embodiments, the nivolumab is administered on days 1 and 15 of a 28 day cycle. In some embodiments, the nivolumab is administered on day 15 of a 21 day cycle. In some embodiments, the nivolumab is administered on days 1 and 15 of a 21 day cycle.

In an embodiment of any of the above embodiments, said other agent is AMP-514. In some embodiments, between about 0.01 mg/kg to about 30 mg/kg of AMP-514 is administered. In some embodiments, between about 0.5 mg/kg to about 30 mg/kg of AMP-514 is administered. In some embodiments, between about 0.5 mg/kg to about 20 mg/kg of AMP-514 is administered. In some embodiments, between about 0.01 mg/kg to about 10 mg/kg of AMP-514 is administered. In some embodiments, between about 0.5 mg/kg to about 10 mg/kg of AMP-514 is administered. In some embodiments, between about 0.1 mg/kg to about 10 mg/kg of AMP-514 is administered. In some embodiments, between about 1 mg/kg to about 10 mg/kg of AMP-514 is administered. In some embodiments, between about 3 mg/kg to about 10 mg/kg of AMP-514 is administered. In some embodiments, about 10 mg/kg of AMP-514 is administered. In some embodiments, at least about 7 mg/kg of AMP-514 is administered. In some embodiments, at least about 5 mg/kg of AMP-514 is administered. In some embodiments, at least about 3 mg/kg of AMP-514 is administered. In some embodiments, at least about 1 mg/kg of AMP-514 is administered. In some embodiments, at least about 0.3 mg/kg of AMP-514 is administered. In some embodiments, the AMP-514 is administered at least once during a cycle. In some embodiments, the AMP-514 is administered at least twice during a cycle. In some embodiments, the AMP-514 is administered at least once a week. In some embodiments, the AMP-514 is administered at least once every two weeks. In some embodiments, the AMP-514 is administered at least once every three weeks. In some embodiments, the AMP-514 is administered at least once every four weeks. In some embodiments, a cycle is 21 days. In some embodiments, a cycle is 28 days. In some embodiments, the AMP-514 is administered on days 1 and 15 of a 28 day cycle. In some embodiments, the AMP-514 is administered on day 15 of a 21 day cycle. In some embodiments, the AMP-514 is administered on days 1 and 15 of a 21 day cycle.

In an embodiment of any of the above embodiments, said other agent is pembrolizumab (MK-3475). In some embodiments, between about 0.1 mg/kg to about 15 mg/kg of pembrolizumab is administered. In some embodiments, between about 0.1 mg/kg to about 10 mg/kg of pembrolizumab is administered. In some embodiments, between about 1 mg/kg to about 15 mg/kg of pembrolizumab is administered. In some embodiments, between about 3 mg/kg to about 12 mg/kg of pembrolizumab is administered. In some embodiments, between about 1 mg/kg to about 10 mg/kg of pembrolizumab is administered. In some embodiments, between about 3 mg/kg to about 10 mg/kg of pembrolizumab is administered. In some embodiments, at least about 1 mg/kg of pembrolizumab is administered. In some embodiments, at least about 2 mg/kg of pembrolizumab is administered. In some embodiments, at least about 3 mg/kg of pembrolizumab is administered. In some embodiments, at least about 5 mg/kg of pembrolizumab is administered. In some embodiments, at least about 10 mg/kg of pembrolizumab is administered. In some embodiments, between about 10 mg to about 400 mg of pembrolizumab is administered. In some embodiments, between about 50 mg to about 400 mg of pembrolizumab is administered. In some embodiments, between about 10 mg to about 300 mg of pembrolizumab is administered. In some embodiments, between about 50 mg to about 300 mg of pembrolizumab is administered. In some embodiments, between about 10 mg to about 250 mg of pembrolizumab is administered. In some embodiments, between about 50 mg to about 250 mg of pembrolizumab is administered. In some embodiments, at least about 50 mg of pembrolizumab is administered. In some embodiments, at least about 100 mg of pembrolizumab is administered. In some embodiments, at least about 150 mg of pembrolizumab is administered. In some embodiments, at least about 200 mg of pembrolizumab is administered. In some embodiments, at least about 250 mg of pembrolizumab is administered. In some embodiments, at least about 300 mg of pembrolizumab is administered. In some embodiments, the pembrolizumab is administered at least once during a cycle. In some embodiments, the pembrolizumab is administered at least twice during a cycle. In some embodiments, a cycle is 21 days. In some embodiments, a cycle is 28 days. In some embodiments, the pembrolizumab is administered at least once a week. In some embodiments, the pembrolizumab is administered at least once every two weeks. In some embodiments, the pembrolizumab is administered at least once every three weeks. In some embodiments, the pembrolizumab is administered at least once every four weeks. In some embodiments, the pembrolizumab is administered on days 1 and 15 of a 28 day cycle. In some embodiments, the pembrolizumab is administered on day 1 of a 21 day cycle. In some embodiments, the pembrolizumab is administered on days 1 and 15 of a 21 day cycle.

In an embodiment of any of the above embodiments, said other agent is pidilizumab (CT-0111). In some embodiments, between about 0.01 mg/kg to about 30 mg/kg of pidilizumab is administered. In some embodiments, between about 0.5 mg/kg to about 30 mg/kg of pidilizumab is administered. In some embodiments, between about 0.5 mg/kg to about 20 mg/kg of pidilizumab is administered. In some embodiments, between about 0.01 mg/kg to about 10 mg/kg of pidilizumab is administered. In some embodiments, between about 0.5 mg/kg to about 10 mg/kg of pidilizumab is administered. In some embodiments, between about 0.1 mg/kg to about 10 mg/kg of pidilizumab is administered. In some embodiments, between about 1 mg/kg to about 10 mg/kg of pidilizumab is administered. In some embodiments, between about 3 mg/kg to about 10 mg/kg of pidilizumab is administered. In some embodiments, about 10 mg/kg of pidilizumab is administered. In some embodiments, at least about 7 mg/kg of pidilizumab is administered. In some embodiments, at least about 5 mg/kg of pidilizumab is administered. In some embodiments, at least about 3 mg/kg of pidilizumab is administered. In some embodiments, at least about 1 mg/kg of pidilizumab is administered. In some embodiments, at least about 0.3 mg/kg of pidilizumab is administered. In some embodiments, the pidilizumab is administered at least once during a cycle. In some embodiments, the pidilizumab is administered at least twice during a cycle. In some embodiments, the pidilizumab is administered at least once a week. In some embodiments, the pidilizumab is administered at least once every two weeks. In some embodiments, the pidilizumab is administered at least once every three weeks. In some embodiments, the pidilizumab is administered at least once every four weeks. In some embodiments, a cycle is 21 days. In some embodiments, a cycle is 28 days. In some embodiments, the pidilizumab is administered on days 1 and 15 of a 28 day cycle. In some embodiments, the pidilizumab is administered on day 15 of a 21 day cycle. In some embodiments, the pidilizumab is administered on days 1 and 15 of a 21 day cycle.

In an embodiment of any of the above embodiments, said other agent is REGN2810. In some embodiments, between about 0.01 mg/kg to about 30 mg/kg of REGN2810 is administered. In some embodiments, between about 0.5 mg/kg to about 30 mg/kg of REGN2810 is administered. In some embodiments, between about 0.5 mg/kg to about 20 mg/kg of REGN2810 is administered. In some embodiments, between about 0.01 mg/kg to about 10 mg/kg of REGN2810 is administered. In some embodiments, between about 0.5 mg/kg to about 10 mg/kg of REGN2810 is administered. In some embodiments, between about 0.1 mg/kg to about 10 mg/kg of REGN2810 is administered. In some embodiments, between about 1 mg/kg to about 10 mg/kg of REGN2810 is administered. In some embodiments, between about 3 mg/kg to about 10 mg/kg of REGN2810 is administered. In some embodiments, about 10 mg/kg of REGN2810 is administered. In some embodiments, at least about 7 mg/kg of REGN2810 is administered. In some embodiments, at least about 5 mg/kg of REGN2810 is administered. In some embodiments, at least about 3 mg/kg of REGN2810 is administered. In some embodiments, at least about 1 mg/kg of REGN2810 is administered. In some embodiments, at least about 0.3 mg/kg of REGN2810 is administered. In some embodiments, the REGN2810 is administered at least once during a cycle. In some embodiments, the REGN2810 is administered at least twice during a cycle. In some embodiments, the REGN2810 is administered at least once a week. In some embodiments, the REGN2810 is administered at least once every two weeks. In some embodiments, the REGN2810 is administered at least once every three weeks. In some embodiments, the REGN2810 is administered at least once every four weeks. In some embodiments, a cycle is 21 days. In some embodiments, a cycle is 28 days. In some embodiments, the REGN2810 is administered on days 1 and 15 of a 28 day cycle. In some embodiments, the REGN2810 is administered on day 15 of a 21 day cycle. In some embodiments, the REGN2810 is administered on days 1 and 15 of a 21 day cycle.

In an embodiment of any of the above embodiments, said other agent is PDR001. In some embodiments, between about 0.01 mg/kg to about 30 mg/kg of PDR001 is administered. In some embodiments, between about 0.5 mg/kg to about 30 mg/kg of PDR001 is administered. In some embodiments, between about 0.5 mg/kg to about 20 mg/kg of PDR001 is administered. In some embodiments, between about 0.01 mg/kg to about 10 mg/kg of PDR001 is administered. In some embodiments, between about 0.5 mg/kg to about 10 mg/kg of PDR001 is administered. In some embodiments, between about 0.1 mg/kg to about 10 mg/kg of PDR001 is administered. In some embodiments, between about 1 mg/kg to about 10 mg/kg of PDR001 is administered. In some embodiments, between about 3 mg/kg to about 10 mg/kg of PDR001 is administered. In some embodiments, about 10 mg/kg of PDR001 is administered. In some embodiments, at least about 7 mg/kg of PDR001 is administered. In some embodiments, at least about 5 mg/kg of PDR001 is administered. In some embodiments, at least about 3 mg/kg of PDR001 is administered. In some embodiments, at least about 1 mg/kg of PDR001 is administered. In some embodiments, at least about 0.3 mg/kg of PDR001 is administered. In some embodiments, the PDR001 is administered at least once during a cycle. In some embodiments, the PDR001 is administered at least twice during a cycle. In some embodiments, the PDR001 is administered at least once a week. In some embodiments, the PDR001 is administered at least once every two weeks. In some embodiments, the PDR001 is administered at least once every three weeks. In some embodiments, the PDR001 is administered at least once every four weeks. In some embodiments, a cycle is 21 days. In some embodiments, a cycle is 28 days. In some embodiments, the PDR001 is administered on days 1 and 15 of a 28 day cycle. In some embodiments, the PDR001 is administered on day 15 of a 21 day cycle. In some embodiments, the PDR001 is administered on days 1 and 15 of a 21 day cycle.

In an embodiment of any of the above embodiments, said other agent is BGB-A317. In some embodiments, between about 0.01 mg/kg to about 30 mg/kg of BGB-A317 is administered. In some embodiments, between about 0.5 mg/kg to about 30 mg/kg of BGB-A317 is administered. In some embodiments, between about 0.5 mg/kg to about 20 mg/kg of BGB-A317 is administered. In some embodiments, between about 0.01 mg/kg to about 10 mg/kg of BGB-A317 is administered. In some embodiments, between about 0.5 mg/kg to about 10 mg/kg of BGB-A317 is administered. In some embodiments, between about 0.1 mg/kg to about 10 mg/kg of BGB-A317 is administered. In some embodiments, between about 1 mg/kg to about 10 mg/kg of BGB-A317 is administered. In some embodiments, between about 3 mg/kg to about 10 mg/kg of BGB-A317 is administered. In some embodiments, about 10 mg/kg of BGB-A317 is administered. In some embodiments, at least about 7 mg/kg of BGB-A317 is administered. In some embodiments, at least about 5 mg/kg of BGB-A317 is administered. In some embodiments, at least about 3 mg/kg of BGB-A317 is administered. In some embodiments, at least about 1 mg/kg of BGB-A317 is administered. In some embodiments, at least about 0.3 mg/kg of BGB-A317 is administered. In some embodiments, the BGB-A317 is administered at least once during a cycle. In some embodiments, the BGB-A317 is administered at least twice during a cycle. In some embodiments, the BGB-A317 is administered at least once a week. In some embodiments, the BGB-A317 is administered at least once every two weeks. In some embodiments, the BGB-A317 is administered at least once every three weeks. In some embodiments, the BGB-A317 is administered at least once every four weeks. In some embodiments, a cycle is 21 days. In some embodiments, a cycle is 28 days. In some embodiments, the BGB-A317 is administered on days 1 and 15 of a 28 day cycle. In some embodiments, the BGB-A317 is administered on day 15 of a 21 day cycle. In some embodiments, the BGB-A317 is administered on days 1 and 15 of a 21 day cycle.

In an embodiment of any of the above embodiments, said other agent is an anti-PD-L1 antibody. In some embodiments, the anti-PD-L1 antibody is selected from the group consisting of BMS-936559, MEDI4736, MSB0010718C and MPDL3280A. In some embodiments, the anti-PD-L1 antibody is BMS-936559. In some embodiments, the anti-PD-L1 antibody is MEDI4736. In some embodiments, the anti-PD-L1 antibody is MPDL3280A. In some embodiments, the anti-PD-L1 antibody is MSB0010718C.

In an embodiment of any of the above embodiments, said other agent is BMS-936559. In some embodiments, between about 0.01 mg/kg to about 30 mg/kg of BMS-936559 is administered. In some embodiments, between about 0.5 mg/kg to about 30 mg/kg of BMS-936559 is administered. In some embodiments, between about 0.5 mg/kg to about 20 mg/kg of BMS-936559 is administered. In some embodiments, between about 0.01 mg/kg to about 10 mg/kg of BMS-936559 is administered. In some embodiments, between about 0.5 mg/kg to about 10 mg/kg of BMS-936559 is administered. In some embodiments, between about 0.1 mg/kg to about 10 mg/kg of BMS-936559 is administered. In some embodiments, between about 1 mg/kg to about 10 mg/kg of BMS-936559 is administered. In some embodiments, between about 3 mg/kg to about 10 mg/kg of BMS-936559 is administered. In some embodiments, about 10 mg/kg of BMS-936559 is administered. In some embodiments, at least about 7 mg/kg of BMS-936559 is administered. In some embodiments, at least about 5 mg/kg of BMS-936559 is administered. In some embodiments, at least about 3 mg/kg of BMS-936559 is administered. In some embodiments, at least about 1 mg/kg of BMS-936559 is administered. In some embodiments, at least about 0.3 mg/kg of BMS-936559 is administered. In some embodiments, the BMS-936559 is administered at least once during a cycle. In some embodiments, the BMS-936559 is administered at least twice during a cycle. In some embodiments, the BMS-936559 is administered at least once a week. In some embodiments, the BMS-936559 is administered at least once every two weeks. In some embodiments, the BMS-936559 is administered at least once every three weeks. In some embodiments, the BMS-936559 is administered at least once every four weeks. In some embodiments, a cycle is 21 days. In some embodiments, a cycle is 28 days. In some embodiments, the BMS-936559 is administered on days 1 and 15 of a 28 day cycle. In some embodiments, the BMS-936559 is administered on day 15 of a 21 day cycle. In some embodiments, the BMS-936559 is administered on days 1 and 15 of a 21 day cycle.

In an embodiment of any of the above embodiments, said other agent is MEDI4736. In some embodiments, between about 1 mg/kg to about 15 mg/kg of MEDI4736 is administered. In some embodiments, between about 3 mg/kg to about 12 mg/kg of MEDI4736 is administered. In some embodiments, between about 1 mg/kg to about 10 mg/kg of MEDI4736 is administered. In some embodiments, at least about 1 mg/kg of MEDI4736 is administered. In some embodiments, at least about 3 mg/kg of MEDI4736 is administered. In some embodiments, at least about 5 mg/kg of MEDI4736 is administered. In some embodiments, at least about 10 mg/kg of MEDI4736 is administered. In some embodiments, the MEDI4736 is administered at least once during a cycle. In some embodiments, between about 100 mg to about 2000 mg of MEDI4736 is administered. In some embodiments, between about 200 mg to about 1800 mg of MEDI4736 is administered. In some embodiments, between about 400 mg to about 1800 mg of MEDI4736 is administered. In some embodiments, between about 600 mg to about 1800 mg of MEDI4736 is administered. In some embodiments, between about 1000 mg to about 1800 mg of MEDI4736 is administered. In some embodiments, between about 1200 mg to about 1800 mg of MEDI4736 is administered. In some embodiments, at least about 500 mg of MEDI4736 is administered. In some embodiments, at least about 800 mg of MEDI4736 is administered. In some embodiments, at least about 1000 mg of MEDI4736 is administered. In some embodiments, at least about 1200 mg of MEDI4736 is administered. In some embodiments, at least about 1400 mg of MEDI4736 is administered. In some embodiments, at least about 1800 mg of MEDI4736 is administered. In some embodiments, the MEDI4736 is administered at least twice during a cycle. In some embodiments, a cycle is 21 days. In some embodiments, a cycle is 28 days. In some embodiments, the MEDI4736 is administered at least once a week. In some embodiments, the MEDI4736 is administered at least once every two weeks. In some embodiments, the MEDI4736 is administered at least once every three weeks. In some embodiments, the MEDI4736 is administered at least once every four weeks. In some embodiments, the MEDI4736 is administered on days 1 and 15 of a 28 day cycle. In some embodiments, the MEDI4736 is administered on day 1 of a 21 day cycle. In some embodiments, the MEDI4736 is administered on days 1 and 15 of a 21 day cycle.

In an embodiment of any of the above embodiments, said other agent is MSB0010718C. In some embodiments, between about 0.01 mg/kg to about 10 mg/kg of MSB0010718C is administered. In some embodiments, between about 1 mg/kg to about 10 mg/kg of MSB0010718C is administered. In some embodiments, between about 3 mg/kg to about 10 mg/kg of MSB0010718C is administered. In some embodiments, about 10 mg/kg of MSB0010718C is administered. In some embodiments, at least about 7 mg/kg of MSB0010718C is administered. In some embodiments, at least about 5 mg/kg of MSB0010718C is administered. In some embodiments, at least about 3 mg/kg of MSB0010718C is administered. In some embodiments, at least about 1 mg/kg of MSB0010718C is administered. In some embodiments, at least about 0.3 mg/kg of MSB0010718C is administered. In some embodiments, the MSB0010718C is administered at least once during a cycle. In some embodiments, the MSB0010718C is administered at least twice during a cycle. In some embodiments, the MSB0010718C is administered at least once a week. In some embodiments, the MSB0010718C is administered at least once every two weeks. In some embodiments, the MSB0010718C is administered at least once every three weeks. In some embodiments, the MSB0010718C is administered at least once every four weeks. In some embodiments, a cycle is 21 days. In some embodiments, a cycle is 28 days. In some embodiments, the MSB0010718C is administered on days 1 and 15 of a 28 day cycle. In some embodiments, the MSB0010718C is administered on day 15 of a 21 day cycle. In some embodiments, the MSB0010718C is administered on days 1 and 15 of a 21 day cycle.

In an embodiment of any of the above embodiments, said other agent is MPDL3280A. In some embodiments, between about 500 to 1500 mg of MPDL3280A is administered. In some embodiments, between about 500 to 1200 mg of MPDL3280A is administered. In some embodiments, between about 600 to 1200 mg of MPDL3280A is administered. In some embodiments, at least about 600 mg of MPDL3280A is administered. In some embodiments, at least about 800 mg of MPDL3280A is administered. In some embodiments, at least about 1000 mg of MPDL3280A is administered. In some embodiments, at least about 1200 mg of MPDL3280A is administered. In some embodiments, at least about 1500 mg of MPDL3280A is administered. In some embodiments, the MPDL3280A is administered at least once during a cycle. In some embodiments, the MPDL3280A is administered at least twice during a cycle. In some embodiments, a cycle is 21 days. In some embodiments, a cycle is 28 days. In some embodiments, the MPDL3280A is administered at least once a week. In some embodiments, the MPDL3280A is administered at least once every two weeks. In some embodiments, the MPDL3280A is administered at least once every three weeks. In some embodiments, the MPDL3280A is administered at least once every four weeks. In some embodiments, the MPDL3280A is administered on days 1 and 15 of a 28 day cycle. In some embodiments, the MPDL3280A is administered on day 1 of a 21 day cycle. In some embodiments, the MPDL3280A is administered on days 1 and 15 of a 21 day cycle.

In an embodiment of any of the above embodiments, the method further comprises administering to said individual at least one chemotherapeutic agent. In some embodiments, the chemotherapeutic agent is nucleoside analog. In some embodiments, the nucleoside analog is gemctabine. In some embodiments, the chemotherapeutic agent is a platinum-based agent. In some embodiments, the platinum-based agent is carboplatin. In some embodiments, the platinum-based agent is cisplatin.

In an embodiment of any of the above embodiments, the method further comprises administering to said individual carboplatin. In some embodiments, the carboplatin is administered at a dosage range between about AUC 1 to about AUC 10. In some embodiments, the carboplatin is administered at a dosage range between about AUC 1 to about AUC 8. In some embodiments, the carboplatin is administered at a dosage range between about AUC 1 to about AUC 6. In some embodiments, the carboplatin is administered at a dose of AUC 1. In some embodiments, the carboplatin is administered at a dose of AUC 2. In some embodiments, the carboplatin is administered at a dose of AUC 3. In some embodiments, the carboplatin is administered at a dose of AUC 4. In some embodiments, the carboplatin is administered at a dose of AUC 5. In some embodiments, the carboplatin is administered at a dose of AUC 6. In some embodiments, the carboplatine is administered at least once during a cycle. In some embodiments, the carboplatin is administered at least twice during a cycle. In some embodiments, the carboplatin is administered at least three times during a cycle. In some embodiments, a cycle is 21 days. In some embodiments, a cycle is 28 days. In some embodiments, a cycle is 21 days. In some embodiments, a cycle is 28 days. In some embodiments, the carboplatin is administered at least once a week. In some embodiments, the carboplatin is administered at least once every two weeks. In some embodiments, the carboplatin is administered at least once every three weeks. In some embodiments, the carboplatin is administered at least once every four weeks. In some embodiments, the carboplatin is administered on day 1 of a 21 day cycle.

In an embodiment of any of the above embodiments, the method further comprises administering to said individual cisplatin. In some embodiments, between about 0.01 mg to about 10 mg of cisplatin is administered. In some embodiments, between about 0.5 mg to about 10 mg of cisplatin is administered. In some embodiments, between about 0.1 mg to about 10 mg of cisplatin is administered. In some embodiments, between about 1 mg to about 10 mg of cisplatin is administered. In some embodiments, between about 3 mg to about 10 mg of cisplatin is administered. In some embodiments, about 10 mg of cisplatin is administered. In some embodiments, at least about 7 mg of cisplatin is administered. In some embodiments, at least about 5 mg of cisplatin is administered. In some embodiments, at least about 3 mg of cisplatin is administered. In some embodiments, at least about 1 mg of cisplatin is administered. In some embodiments, at least about 0.3 mg of cisplatin is administered. In some embodiments, the cisplatin is administered at least once during a cycle. In some embodiments, the cisplatin is administered at least twice during a cycle. In some embodiments, the cisplatin is administered at least once a week. In some embodiments, the cisplatin is administered at least once every two weeks. In some embodiments, the cisplatin is administered at least once every three weeks. In some embodiments, the cisplatin is administered at least once every four weeks. In some embodiments, a cycle is 21 days. In some embodiments, a cycle is 28 days. In some embodiments, the cisplatin is administered on days 1 and 15 of a 28 day cycle. In some embodiments, the cisplatin is administered on day 15 of a 21 day cycle. In some embodiments, the cisplatin is administered on days 1 and 15 of a 21 day cycle.

In an embodiment of any of the above embodiments, the method further comprises administering to said individual gemcitabine. In some embodiments, between about 500 to 1500 mg of gemcitabine is administered. In some embodiments, between about 500 to 1200 mg of gemcitabine is administered. In some embodiments, between about 600 to 1200 mg of gemcitabine is administered. In some embodiments, at least about 600 mg of gemcitabine is administered. In some embodiments, at least about 800 mg of gemcitabine is administered. In some embodiments, at least about 1000 mg of gemcitabine is administered. In some embodiments, at least about 1200 mg of gemcitabine is administered. In some embodiments, the gemcitabine is administered at least once during a cycle. In some embodiments, the gemcitabine is administered at least twice during a cycle. In some embodiments, the gemcitabine is administered at least three times during a cycle. In some embodiments, a cycle is 21 days. In some embodiments, a cycle is 28 days. In some embodiments, a cycle is 21 days. In some embodiments, a cycle is 28 days. In some embodiments, the gemcitabine is administered at least once a week. In some embodiments, the gemcitabine is administered at least once every two weeks. In some embodiments, the gemcitabine is administered at least once every three weeks. In some embodiments, the gemcitabine is administered at least once every four weeks. In some embodiments, the gemcitabine is administered on days 1, 8 and 15 of a 28 day cycle. In some embodiments, the gemcitabine is administered on day 1 of a 21 day cycle.

In a further embodiment of the above embodiments, the proliferative disease is cancer. In a further embodiment, the cancer is breast cancer. In yet a further embodiment, the individual is negative for ER, PR, or HER2. In yet a further embodiment, the individual is negative for HER2. In a further embodiment, the individual is negative for ER and HER2. In a further embodiment, the individual is negative for PR and HER2. In a further embodiment, the individual is negative for ER and PR. In a further embodiment, the individual is negative for ER, PR, and HER2.

In a further embodiment of the above embodiments, the cancer is breast cancer. In a further embodiment of the above embodiments, the cancer is a metastatic breast cancer.

In a further embodiment of the above embodiments, the cancer is lung cancer. In a further embodiment of the above embodiments, the cancer is non-small lung cancer.

In a further embodiment of the above embodiments, the cancer is pancreatic cancer. In some embodiments, the pancreatic cancer is locally advanced, surgically resected or unresected pancreatic cancer or metastatic pancreatic adenocarcinoma.

In an embodiment of any of the above embodiments, the composition comprising nanoparticles comprising a taxane (e.g., paclitaxel) and a carrier protein (e.g., albumin) and the other agent are administered simultaneously.

In an embodiment of any of the above embodiments, the composition comprising nanoparticles of taxane (e.g., paclitaxel) and a carrier protein (e.g., albumin) and the other agent are administered sequentially.

In another embodiment of any of the above embodiments, the composition comprising nanoparticles of taxane (e.g., paclitaxel) and a carrier protein (e.g., albumin) and the other agent are administered concurrently.

In an embodiment of any of the above embodiments, the composition comprising nanoparticles comprising a taxane (e.g., paclitaxel) and a carrier protein (e.g., albumin) and the chemotherapeutic agent are administered simultaneously.

In an embodiment of any of the above embodiments, the composition comprising nanoparticles of taxane (e.g., paclitaxel) and a carrier protein (e.g., albumin) and the chemotherapeutic agent are administered sequentially.

In another embodiment of any of the above embodiments, the composition comprising nanoparticles of taxane (e.g., paclitaxel) and carrier protein (e.g., albumin) and the chemotherapeutic agent are administered concurrently.

In an embodiment of any of the above embodiments, the chemotherapeutic agent and the other agent are administered simultaneously.

In an embodiment of any of the above embodiments, the chemotherapeutic agent and the other agent are administered sequentially.

In another embodiment of any of the above embodiments, the chemotherapeutic agent and the other agent are administered concurrently.

In an embodiment of any of the above embodiments, the taxane is paclitaxel.

In an embodiment of any of the above embodiments, the amount of taxane (e.g., paclitaxel) administered to the individual is about 45 mg/m² to about 350 mg/m². In an embodiment of any of the above embodiments, the amount of taxane (e.g., paclitaxel) administered to the individual is about 50 mg/m² to about 300 mg/m². In an embodiment of any of the above embodiments, the amount of taxane (e.g., paclitaxel) administered to the individual is about 50 mg/m² to about 350 mg/m². In an embodiment of any of the above embodiments, the amount of taxane (e.g., paclitaxel) administered to the individual is about 60 mg/m² to about 300 mg/m². In an embodiment of any of the above embodiments, the amount of taxane (e.g., paclitaxel) administered to the individual is between about 75 mg/m² to about 275 mg/m². In an embodiment of any of the above embodiments, the amount of taxane (e.g., paclitaxel) administered to the individual is between about 80 mg/m² to about 350 mg/m². In an embodiment of any of the above embodiments, the amount of taxane (e.g., paclitaxel) administered to the individual is between about 80 mg/m² to about 300 mg/m². In an embodiment of any of the above embodiments, the amount of taxane (e.g., paclitaxel) administered to the individual is about 80 mg/m² to about 200 mg/m². In an embodiment of any of the above embodiments, the amount of taxane (e.g., paclitaxel) administered to the individual is between about 80 mg/m² to about 150 mg/m². In an embodiment of any of the above embodiments, the amount of taxane (e.g., paclitaxel) administered to the individual is between about 100 mg/m² to about 250 mg/m². In an embodiment of any of the above embodiments, the amount of taxane (e.g., paclitaxel) administered to the individual is between about 150 mg/m² to about 350 mg/m². In an embodiment of any of the above embodiments, the amount of taxane (e.g., paclitaxel) administered to the individual is between about 170 mg/m² to about 200 mg/m². In an embodiment of any of the above embodiments, the amount of taxane (e.g., paclitaxel) administered to the individual is between about 200 mg/m² to about 350 mg/m². In an embodiment of any of the above embodiments, the amount of taxane (e.g., paclitaxel) administered to the individual is about 50 mg/m². In an embodiment of any of the above embodiments, the amount of taxane (e.g., paclitaxel) administered to the individual is about 75 mg/m². In an embodiment of any of the above embodiments, the amount of taxane (e.g., paclitaxel) administered to the individual is about 100 mg/m². In an embodiment of any of the above embodiments, the amount of taxane (e.g., paclitaxel) administered to the individual is about 125 mg/m². In an embodiment of any of the above embodiments, the amount of taxane (e.g., paclitaxel) administered to the individual is about 150 mg/m². In an embodiment of any of the above embodiments, the amount of taxane (e.g., paclitaxel) administered to the individual is about 175 mg/m². In an embodiment of any of the above embodiments, the amount of taxane (e.g., paclitaxel) administered to the individual is about 200 mg/m². In an embodiment of any of the above embodiments, the amount of taxane (e.g., paclitaxel) administered to the individual is about 225 mg/m². In an embodiment of any of the above embodiments, the amount of taxane (e.g., paclitaxel) administered to the individual is about 250 mg/m². In an embodiment of any of the above embodiments, the amount of taxane (e.g., paclitaxel) administered to the individual is about 260 mg/m². In an embodiment of any of the above embodiments, the amount of taxane (e.g., paclitaxel) administered to the individual is about 275 mg/m². In an embodiment of any of the above embodiments, the amount of taxane (e.g., paclitaxel) administered to the individual is about 300 mg/m². In an embodiment of any of the above embodiments, the amount of taxane (e.g., paclitaxel) administered to the individual is about 325 mg/m². In an embodiment of any of the above embodiments, the amount of taxane (e.g., paclitaxel) administered to the individual is about 350 mg/m². In an embodiment of any of the above embodiments, the taxane (e.g., paclitaxel) is administered at least once during a cycle. In an embodiment of any of the above embodiments, the taxane (e.g., paclitaxel) is administered at least twice during a cycle. In an embodiment of any of the above embodiments, the taxane (e.g., paclitaxel) is administered at least three times during a cycle. In an embodiment of any of the above embodiments, the taxane (e.g., paclitaxel) is administered at least once a week. In an embodiment of any of the above embodiments, the nab-paclitaxel (e.g., ABRAXANE®) is administered at least once every two weeks. In an embodiment of any of the above embodiments, the taxane (e.g., paclitaxel) is administered at least once every three weeks. In an embodiment of any of the above embodiments, the taxane (e.g., paclitaxel) is administered at least once every four weeks. In an embodiment of any of the above embodiments, a cycle is 21 days. In an embodiment of any of the above embodiments, a cycle is 28 days. In an embodiment of any of the above embodiments, the taxane (e.g., paclitaxel) is administered on days 1, 8 and 15 of a 28 day cycle. In an embodiment of any of the above embodiments, the taxane (e.g., paclitaxel) is administered on day 1 of a 21 day cycle. In an embodiment of any of the above embodiments, the taxane (e.g., paclitaxel) is administered on days 1, 8 and 15 of a 21 day cycle.

In an embodiment of any of the above embodiments, the nanoparticle comprising the taxane and albumin is nab-paclitaxel. In some embodiments, the nab-paclitaxel is ABRAXANE®.

In an embodiment of any of the above embodiments, the amount of nab-placitaxel (e.g., ABRAXANE®) administered to the individual is about 45 mg/m² to about 350 mg/m². In an embodiment of any of the above embodiments, the amount of nab-placitaxel (e.g., ABRAXANE®) administered to the individual is about 50 mg/m² to about 300 mg/m². In an embodiment of any of the above embodiments, the amount of nab-placitaxel (e.g., ABRAXANE®) administered to the individual is about 50 mg/m² to about 350 mg/m². In an embodiment of any of the above embodiments, the amount of nab-placitaxel (e.g., ABRAXANE®) administered to the individual is about 60 mg/m² to about 300 mg/m². In an embodiment of any of the above embodiments, the amount of nab-placitaxel (e.g., ABRAXANE®) administered to the individual is between about 75 mg/m² to about 275 mg/m². In an embodiment of any of the above embodiments, the amount of nab-placitaxel (e.g., ABRAXANE®) administered to the individual is between about 80 mg/m² to about 350 mg/m². In an embodiment of any of the above embodiments, the amount of nab-placitaxel (e.g., ABRAXANE®) administered to the individual is between about 80 mg/m² to about 300 mg/m². In an embodiment of any of the above embodiments, the amount of nab-placitaxel (e.g., ABRAXANE®) administered to the individual is about 80 mg/m² to about 200 mg/m². In an embodiment of any of the above embodiments, the amount of nab-placitaxel (e.g., ABRAXANE®) administered to the individual is between about 80 mg/m² to about 150 mg/m². In an embodiment of any of the above embodiments, the amount of nab-placitaxel (e.g., ABRAXANE®) administered to the individual is between about 100 mg/m² to about 250 mg/m². In an embodiment of any of the above embodiments, the amount of nab-placitaxel (e.g., ABRAXANE®) administered to the individual is between about 150 mg/m² to about 350 mg/m². In an embodiment of any of the above embodiments, the amount of nab-placitaxel (e.g., ABRAXANE®) administered to the individual is between about 170 mg/m² to about 200 mg/m². In an embodiment of any of the above embodiments, the amount of nab-placitaxel (e.g., ABRAXANE®) administered to the individual is between about 200 mg/m² to about 350 mg/m². In an embodiment of any of the above embodiments, the amount of nab-placitaxel (e.g., ABRAXANE®) administered to the individual is about 50 mg/m². In an embodiment of any of the above embodiments, the amount of nab-placitaxel (e.g., ABRAXANE®) administered to the individual is about 75 mg/m². In an embodiment of any of the above embodiments, the amount of nab-placitaxel (e.g., ABRAXANE®) administered to the individual is about 100 mg/m². In an embodiment of any of the above embodiments, the amount of nab-placitaxel (e.g., ABRAXANE®) administered to the individual is about 125 mg/m². In an embodiment of any of the above embodiments, the amount of nab-placitaxel (e.g., ABRAXANE®) administered to the individual is about 150 mg/m². In an embodiment of any of the above embodiments, the amount of nab-placitaxel (e.g., ABRAXANE®) administered to the individual is about 175 mg/m². In an embodiment of any of the above embodiments, the amount of nab-placitaxel (e.g., ABRAXANE®) administered to the individual is about 200 mg/m². In an embodiment of any of the above embodiments, the amount of nab-placitaxel (e.g., ABRAXANE®) administered to the individual is about 225 mg/m². In an embodiment of any of the above embodiments, the amount of nab-placitaxel (e.g., ABRAXANE®) administered to the individual is about 250 mg/m². In an embodiment of any of the above embodiments, the amount of nab-placitaxel (e.g., ABRAXANE®) administered to the individual is about 260 mg/m². In an embodiment of any of the above embodiments, the amount of nab-placitaxel (e.g., ABRAXANE®) administered to the individual is about 275 mg/m². In an embodiment of any of the above embodiments, the amount of nab-placitaxel (e.g., ABRAXANE®) administered to the individual is about 300 mg/m². In an embodiment of any of the above embodiments, the amount of nab-placitaxel (e.g., ABRAXANE®) administered to the individual is about 325 mg/m². In an embodiment of any of the above embodiments, the amount of nab-placitaxel (e.g., ABRAXANE®) administered to the individual is about 350 mg/m². In an embodiment of any of the above embodiments, the nab-paclitaxel (e.g., ABRAXANE®) is administered at least once during a cycle. In an embodiment of any of the above embodiments, the nab-paclitaxel (e.g., ABRAXANE®) is administered at least twice during a cycle. In an embodiment of any of the above embodiments, the nab-paclitaxel (e.g., ABRAXANE®) is administered at least three times during a cycle. In an embodiment of any of the above embodiments, the nab-paclitaxel (e.g., ABRAXANE®) is administered at least once a week. In an embodiment of any of the above embodiments, the nab-paclitaxel (e.g., ABRAXANE®) is administered at least once every two weeks. In an embodiment of any of the above embodiments, the nab-paclitaxel (e.g., ABRAXANE®) is administered at least once every three weeks. In an embodiment of any of the above embodiments, the nab-paclitaxel (e.g., ABRAXANE®) is administered at least once every four weeks. In an embodiment of any of the above embodiments, a cycle is 21 days. In an embodiment of any of the above embodiments, a cycle is 28 days. In an embodiment of any of the above embodiments, the nab-paclitaxel (e.g., ABRAXANE®) is administered on days 1, 8 and 15 of a 28 day cycle. In an embodiment of any of the above embodiments, the nab-paclitaxel (e.g., ABRAXANE®) is administered on day 1 of a 21 day cycle. In an embodiment of any of the above embodiments, the nab-paclitaxel (e.g., ABRAXANE®) is administered on days 1, 8 and 15 of a 21 day cycle.

In an embodiment of any of the above embodiments, the average diameter of the nanoparticles in the composition is no greater than about 200 nm.

In an embodiment of any of the above embodiments, the carrier protein is albumin. In a further embodiment, the weight ratio of the albumin to the taxane in the nanoparticle composition is between about 1:1 to about 18:1. In a further embodiment, the weight ratio of the albumin to the taxane in the nanoparticle composition is between about 1:1 to about 9:1. In a further embodiment, the weight ratio of the albumin to the taxane in the nanoparticle composition is about 9:1.

In an embodiment of any of the above embodiments, the individual is a human.

In another aspect, provided herein is a kit comprising: a) a composition comprising nanoparticles comprising a taxane (e.g., paclitaxel) and a carrier protein (e.g., albumin), and b) an effective amount of at least one other agent that antagonizes a PD-1 pathway in a cell.

In another aspect, provided herein is a pharmaceutical composition comprising: a) a composition comprising nanoparticles comprising a taxane (e.g., paclitaxel) and a carrier protein (e.g., albumin), and b) an effective amount of at least one other agent that antagonizes PD-1.

In another aspect, provided herein is a pharmaceutical composition comprising: a) a composition comprising nanoparticles comprising a taxane (e.g., paclitaxel) and a carrier protein (e.g., albumin), and b) an effective amount of at least one other agent that antagonizes PD-L1.

In another aspect, provided herein is a pharmaceutical composition comprising: a) a composition comprising nanoparticles comprising a taxane (e.g., paclitaxel) and a carrier protein (e.g., albumin), and b) an effective amount of at least one other agent that antagonizes PD-L1.

In an embodiment of any of the above embodiments or aspects, the taxane is paclitaxel.

In an embodiment of any of the above embodiments or aspects, the carrier protein is albumin.

In an embodiment of any of the above embodiments or aspects, the nanoparticle comprising the taxane and the carrier protein is nab-paclitaxel. In an embodiment of any of the above embodiments or aspects, the nab-paclitaxel is ABRAXANE®.

In an embodiment of any of the above embodiments or aspects, the other agent is an antibody.

In an embodiment of any of the above embodiments or aspects, the other agent is a monoclonal antibody.

In an embodiment of any of the above embodiments or aspects, the other agent is a human antibody.

In an embodiment of any of the above embodiments or aspects, the other agent is a humanized antibody.

In an embodiment of any of the above embodiments or aspects, the other agent is a chimeric antibody.

In an embodiment of any of the above embodiments or aspects, the other agent comprises at least a portion of an immunoglobulin (Ig).

In an embodiment of any of the above embodiments or aspects, the other agent comprises at least a portion of an immunoglobulin G (IgG).

In an embodiment of any of the above embodiments or aspects, the other agent comprises at least a portion of an immunoglobulin G4 (IgG4).

Those skilled in the art will recognize that several embodiments are possible within the scope and spirit of this disclosure. The following non-limiting examples further illustrate the disclosure but, of course, should not be construed as in any wayscope-limiting.

### EXAMPLES

### Example 1. Nab-Paclitaxel with Nivolumab (BMS-936558) in Metastatic Breast Cancer (mBC)

A Phase 1, open-label, multicenter, safety study of nab-paclitaxel based chemotherapy regimens administered prior to and/or in combination with nivolumab in metastatic breast cancer (mBC; in particular, HER-2-negative recurrent metastatic breast cancer after one prior regimen for mBC, including an anthracycline unless clinically contraindicated) is performed.

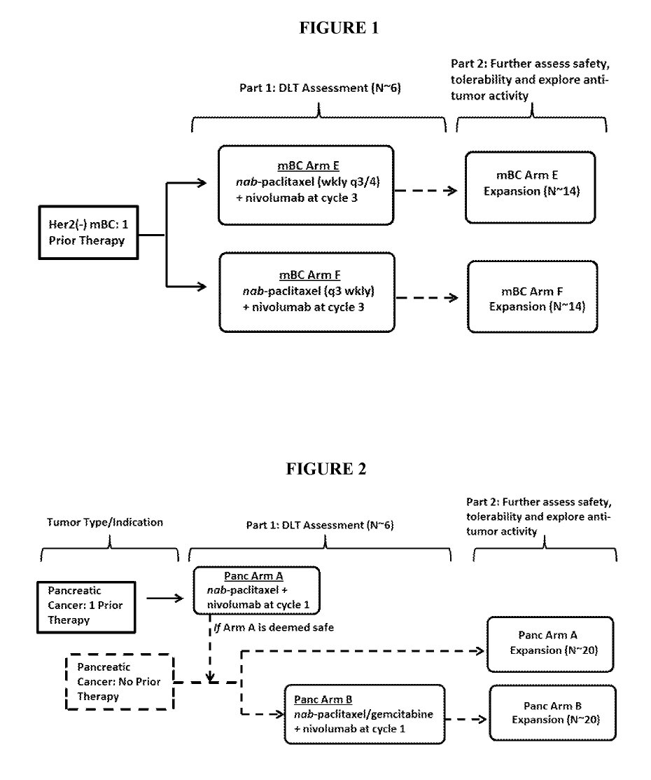

This study includes two treatment arms (mBC Arm E and F). In this study, subjects in mBC Arm E receives weekly nab-paclitaxel with nivolumab starting at Cycle 3, with nab-paclitaxel on days 1, 8 and 15 of each 28 day cycle, plus nivolumab on days 1 and 15 starting in Cycle 3. Subjects in mBC Arm F receive q3 weekly nab-paclitaxel with nivolumab starting at Cycle 3, with nab-paclitaxel on Day 1 of each 21 day cycle, and nivolumab on Day 15 starting in Cycle 3. The study design in shown in FIG. 1.

Objectives:

The primary objective of the study is to evaluate the safety of nab-paclitaxel therapies prior to and/or in mBC. The primary objective is to evaluate the dose limiting toxicities (DLTs) of each combination regimen and to characterize the safety profile of each combination regimen. The secondary objectives are to explore the anti-tumor activity and assess the safety and tolerability of the regimens.

Endpoints:

The primary endpoints of the study are the number of subjects with dose limiting toxicities (DLTs) in each treatment arm (Part 1), and the percentage of subjects with Grade 3 or 4 treatment emergent adverse events (TEAEs) or treatment discontinuation due to a TEAE during the study (Parts 1 and 2). The secondary endpoints of the study are TEACs leading to dose reduction, delay, interruption or treatment discontinuation; progression-free survival (based on Response Evaluation Criteria in Solid Tumors (RECIST) 1.1, as assessed by the investigator); overall survival′ disease control rate (stable disease or better based on RECIST 1.1); overall response rate (based on RECIST 1.1); and duration of response (based on RECIST 1.1).

Study Design:

Eligible subjects are randomly assigned randomly between treatment arms E and F. An interactive response technology (IRT) system is used to ensure the central randomization of subjects.

DLTs are assessed during the first 2 cycles of nivolumab. Subjects are enrolled in each treatment arm to ensure up to 6 evaluable subjects in each arm at the given dose level (DL) for assessment of DLT. If ≦1 DLT occurs at a given DL in 6 subjects, and the DL is deemed safe by the Data Review Committee (DRC), this dose will be the recommended Part 2 dose (RP2D).

There are no dose escalation of nivolumab in any treatment arm. If >1 DLT occurs with a DL within a treatment arm and the DL is deemed unsafe by the DRC, the dose of nivolumab may be de-escalated within a treatment arm for further evaluation. The DRC is responsible for evaluating the safe dose, and for safeguarding the interests of study participants and monitoring the overall conduct of the study.

In each mBC treatment arm, a minimum of 9 triple-negative breast cancer (TNBC) subjects are enrolled to allow for exploration of the safety and efficacy in this subset of subjects independently, and in addition to the assessment in hormone-receptor positive, HER2(−) mBC subjects.

Subjects are treated with nivolumab at the given dose level administered intravenously over 60 minutes prior to chemotherapy according to protocol-defined treatment regimen. nab-Paclitaxel, should be stored, reconstituted, and administered according to the local prescribing information, at the protocol defined dose and frequency.

Subjects receive treatment according to their assigned treatment arm with nivolumab and a fixed starting dose of chemotherapy. The following combination regimens are administered:

mBC Arms E and F: parallel enrollment in Parts 1 and 2
a. mBC Arm E: nab-paclitaxel 100 mg/m2 on Days 1, 8 and 15 of each 28 day cycle, plus nivolumab on Days 1 and 15 starting in Cycle 3.
b. mBC Arm F: nab-paclitaxel 260 mg/m2 on Days 1 of each 21 day cycle, plus nivolumab on Day 15 starting in Cycle 3.

In mBC treatment arms, nivolumab is given at 3 mg/kg q2 weekly in 28 day cycles (mBC Arm E) or 5 mg/kg q3 weekly in 21 day cycles (mBC Arm F) as dose level 1 (DL1), and if needed, de-escalated to a 1 mg/kg q3 weekly or 3 mg/kg q2 weekly dose level (DL-1), respectively. If further reduction of nivolumab is needed in a treatment arm, additional de-escalation to 0.3 mg/kg q3 weekly and 1 mg/kg q2 weekly dose level (DL-2), respectively, may occur. The dose levels for mBC Arms E and F are shown in Table 1.

TABLE 1

| mBC | Dose | nab-Paclitaxel, starting | |
|---|---|---|---|
| Arm | Level | cycle 1 | Nivolumab, starting cycle 3 |
| E | 1 | 260 mg/m² D 1, 8, 15 | 3 mg/kg, D 1, 15 of 28 day |
| | | of 28 day cycle | cycle |
| | −1 | 260 mg/m² D 1,8, 15 | 1 mg/kg, D 1, 15 of 28 day |
| | | of 28 day cycle | cycle |
| | −2 | 260 mg/m² D 1, 8, 15 | 0.3 mg/kg, D 1, 15 of 28 day |
| | | of 28 day cycle | cycle |
| F | 1 | 100 mg/m² D 1 | 5 mg/kg, D 15 of 21 day cycle |
| | | of 21 day cycle | |
| | −1 | 100 mg/m² D 1 | 3 mg/kg, D 15 of 21 day cycle |
| | | of 21 day cycle | |
| | −2 | 100 mg/m² D 1 | 1 mg/kg, D 15 of 21 day cycle |
| | | of 21 day cycle | |

D = day

Treatment regimens are given until RECIST 1.1 disease progression, unacceptable toxicity or withdrawal of consent. Subjects will be permitted to continue nivolumab treatment beyond initial RECIST 1.1 defined progressive disease as long as they meet the following criteria:

Continue to meet all other study protocol eligibility criteria

Investigator assessed clinical benefit and do not have rapid disease progression or clinical deterioration

Stable performance status

Tolerance to study drug

Treatment beyond progression will not delay an imminent intervention to prevent serious complications of disease progression (eg, brain metastases).

Subjects:

The study enrolls approximately 36 to 138 subjects with histologically or cytologically confirmed cancer, including mBC (Human Epidermal Growth Factor Receptor 2-negative [HER2(−)] recurrent metastatic breast cancer after one prior regimen for metastatic disease, including an anthracycline unless clinically contraindicated).

Overview of Efficacy Assessments:

All subjects are evaluated for tumor response and progression according to RECIST 1.1 guidelines (investigator assessment). Baseline tumor measurements are determined from the radiologic evaluation performed within 28 days before Cycle 1 Day 1. Thereafter, radiologic evaluation is performed every 6 weeks (±3 days) from Cycle 1 Day 1 for the first 24 weeks (first 4 assessments) and then every 8 weeks until disease progression, start of a new anticancer therapy or withdrawal of consent from the entire study, whichever comes first. Those subjects entering the follow-up period continue to have CT scans in accordance with local standard of care until withdrawal of consent, lost to follow-up, death, or termination of the study by the sponsor. In the follow-up period, anti-cancer treatment administered following the last dose of investigational product ([IP]: nab-paclitaxel or nivolumab) and survival is followed every 3 months (±7 days), by telephone or subject visit, or review of records until death or withdrawal of consent. Subjects are followed for overall survival for up to 1 year after the last subject is enrolled.

Overview of Safety Assessments:

All subjects are monitored for adverse events, starting from the time the subject signs the informed consent form. Treatment-emergent adverse events (TEAEs) are captured through 28 days after the last dose of the IPs. In addition, any AE with an onset date more than 28 days and up to 100 days after the last dose of the IPs is also considered a TEAE. Physical examination (source documented only), vital signs, laboratory assessments (eg, serum chemistry, hematology), and ECOG performance status are monitored regularly. Preventative measures are taken to avoid pregnancy in trial subjects or their partners, and females of child-bearing potential will have regular pregnancy testing. DLTs are assessed during the first two cycles of nivolumab. In the follow-up period, safety continues to be followed, either via phone or office visit, every 30 days (±7 days) and on Day 100 for adverse events. Laboratory assessment of complete blood counts and serum chemistry is performed at Day 60 (±7 days) and Day 100 (±7 days).

Study Population—Inclusion Criteria:

Subjects must satisfy the following criteria to be enrolled in both Part 1 and Part 2 of the study:

1. Subject is male or female, ≧18 years old at the time of signing the informed consent form (ICF).

2. Subject has a confirmed diagnosis of advanced unresectable solid tumors in the target subject population within the parameters mentioned:

mBC Arms E and F (HER2(−) recurrent metastatic Breast Cancer):
Subject has a definitive histologically or cytologically confirmed diagnosis of HER2(−) metastatic breast cancer.
Subject has received one prior cytotoxic chemotherapy regimen for metastatic disease, including an anthracycline unless clinically contraindicated. (Clinically contraindicated is defined as unless: [a.] anthracycline treatment was not indicated or was not the best treatment option for the subject in the opinion of the treating physician; and [b.] anthracycline treatment remains not indicated or, in the opinion of the treating physician, is not the best treatment option for the subject's metastatic disease.)
If subject has received solvent-based paclitaxel (TAXOL) or docetaxel as adjuvant chemotherapy, subject must not have relapsed with breast cancer within 12 months of completing said therapy.
Suitable candidate for single agent nab-paclitaxel as assessed by the investigator. Subject has measurable disease according to RECIST 1.1.

3. Subject has Eastern Cooperative Oncology Group (ECOG) performance status 0 or 1.

4. Subject has no other malignancy within 5 years, except non-melanoma skin cancer, cervical intraepithelial neoplasia, or in-situ cervical cancer.

5. Subject has the following laboratory values at screening:

WBCs≧2000/uL,

Absolute neutrophil count (ANC)≧1.5×109/L,

Hemoglobin (Hgb)≧90 g/L,

Platelets (plt)≧100×109/L,

Potassium within normal range, or correctable with supplements,

AST and ALT≦2.5× Upper Limit of Normal (ULN) or ≦3.0×ULN if liver tumor is present,

Serum total bilirubin≦1.5×ULN (except in subjects with Gilbert's who may have serum bilirubin≦3.0×ULN),

Serum creatinine≦1.5×ULN, or 24-hr clearance≧60 mL/min,

Normal coagulation [prothrombin time and partial thromboplastin time within normal limits (±15%)].

6. Subject has resting baseline oxygen saturation by pulse oximetry of ≧92% at rest.

7. Females of child-bearing potential (defined as a sexually mature woman who: 1) has not undergone a hysterectomy (the surgical removal of the uterus) or bilateral oophorectomy (the surgical removal of both ovaries) or, 2) has not been naturally postmenopausal for at least 24 consecutive months (ie, has had menses at any time during the preceding 24 consecutive months) must:

Agree to use, and be able to comply with, highly effective contraception (failure rate less than 1% per year) without interruption while on study treatment and for 23 weeks after discontinuation; and

Have a negative serum pregnancy test result (minimum sensitivity 25 IU/L or equivalent units of β-hCG) at screening and 24 hours prior to the start of any IP and agree to ongoing pregnancy testing during the course of the study, and after the end of study therapy.

Women must not be breastfeeding. (Females should not be breastfeeding while receiving nivolumab and up to 18 weeks from the last dose of nivolumab).

8. Male subjects agree to use a condom during sexual contact with a pregnant female or a female of childbearing potential while participating in the study, during dose interruptions and for 31 weeks following IP discontinuation, even if he has undergone a successful vasectomy.

9. Subject or his/her legally authorized representative or guardian understands and voluntarily signs an informed consent document prior to any study related assessments/procedures are conducted.

10. Subject is able to adhere to the study visit schedule and other protocol requirements.

Study Population—Exclusion Criteria:

The presence of any of the following will exclude a subject from enrollment:

1. Subject has a history of allergy or hypersensitivity to any study drugs or their excipients.

2. Subject has symptomatic brain metastases, spinal cord compression, or intractable back pain due to compression of destructive mass.

3. Subject has active, known or suspected autoimmune disease, including systemic lupus erythematodes, Hashimotos thyroiditis, scleroderma, polyarteritis nodosa or auto-immune hepatitis. Subjects with Type I diabetes mellitus, hypothyroidism only requiring hormone replacement, skin disorders (such as vitiligo, psoriasis or alopecia) not requiring systemic treatment, or conditions not expected to recur in the absence of an external trigger are permitted to enroll.

4. Subject is currently receiving or requires treatment with immunosuppressive agents or immunosuppressive doses of systemic corticosteroids (unless used to treat drug-related adverse events). Topical, ocular, intra-articular, intranasal, inhalational corticosteroids (with minimal systemic absorption), and some uses of systemic corticosteroids may be permitted.

5. Subject has any peripheral neuropathy≧NCI CTCAE Grade 2 at randomization/enrollment.

6. Subject has a history of interstitial lung disease, history of slowly progressive dyspnea and unproductive cough, sarcoidosis, silicosis, idiopathic pulmonary fibrosis, pulmonary hypersensitivity pneumonitis or multiple allergies. Any lung disease that may interfere with the detection or management of suspected drug-related pulmonary toxicity.

7. Subject has a high cardiovascular risk, including, but not limited to, recent coronary stenting or myocardial infarction in the past year.

8. Subject has unstable angina, a significant cardiac arrhythmia, or New York Heart Association Class 3 or 4 congestive heart failure.

9. Subject has a history of peripheral artery disease (eg, claudication, Leo Buerger's disease).

10. Subject has had major surgery, other than diagnostic surgery, within 4 weeks prior to treatment in study.

11. Subject has known acute or chronic pancreatitis.

12. Subject has persistent diarrhea, malabsorption, or known sub-acute bowel obstruction≧NCI CTCAE Grade 2, despite medical management.

13. Subject has active, uncontrolled bacterial, viral, or fungal infection(s) requiring systemic therapy.

14. Subject has any history of testing positive for Human Immunodeficiency Virus (HIV) or known acquired immunodeficiency disorder (AIDS).

15. Subject has historical or active infection with hepatitis B, or hepatitis C.

16. Subject is pregnant or breast-feeding.

17. Subject is currently enrolled in any other clinical protocol or investigational trial that involves administration of experimental therapy and/or therapeutic devices, or investigational drug.

18. Subject is currently using or use within 6 months of illicit drugs.

19. Subject has any significant medical condition, laboratory abnormality, or psychiatric illness that would prevent the subject from participating in the study.

20. Subject has any condition, including the presence of laboratory abnormalities, which places the subject at unacceptable risk if he/she were to participate in the study.

21. Subject has any condition that confounds the ability to interpret data from the study.

### Example 2. Nab-Paclitaxel with Nivolumab (BMS-936558) with and without Gemcitabine in Pancreatic Cancer

A Phase 1, open-label, multicenter, safety study of nab-paclitaxel based chemotherapy regimens administered prior to and/or in combination with nivolumab in pancreatic cancer is performed. This study includes two parts (Part 1 and 2) and two pancreatic cancer arms (Panc Arm A and B). In Part 1 of this study, subjects in Panc Arm A receive nab-paclitaxel in combination with nivolumab. In Part 2 of this study, if Panc Arm A is deemed safe, subjects in Panc Arm B are administered nab-paclitaxel, nivolumab and gemcitabine. The study design in shown in FIG. 2.

Objectives:

The primary objective of the study is to evaluate the safety of nab-paclitaxel therapies in combination with nivolumab and, optionally, with gemcitabine administered at different doses and treatment schedules in pancreatic cancer. Enrollment in each treatment arm is conducted in two sequential parts to allow for the evaluation of the Part 1 and 2 study objectives. In Part 1, the primary objective is to evaluate the dose limiting toxicities (DLTs) of each combination regimen. In Part 1 and Part 2 combined, the primary objective is to further characterize the safety profile of each combination regimen. For both Part 1 and Part 2, the secondary objectives are to explore the anti-tumor activity and assess the safety and tolerability of the proposed regimens for each of the tumor types/indications.

Study Design: Part 1 of this study first evaluates the incidence of DLTs of the selected nivolumab doses in combination with the standard nab-paclitaxel therapies. Subjects who meet the study entry criteria are assigned to a treatment arm based on tumor type and indication. Each tumor type/indication has two treatment arms. Panc Arms A and B are initiated sequentially in Part 1. Panc Arm B, does not start until Panc Arm A is deemed safe in Part 1.

DLTs are assessed during the first 2 cycles of nivolumab. Subjects are enrolled in each treatment arm to ensure up to 6 evaluable subjects in each arm at the given dose level (DL) for assessment of DLT. If ≦1 DLT occurs at a given DL in 6 subjects, and the DL is deemed safe by the Data Review Committee (DRC), this dose will be the recommended Part 2 dose (RP2D).

There are no dose escalation of nivolumab in any treatment arm. If >1 DLT occurs with a DL within a treatment arm and the DL is deemed unsafe by the DRC, the dose of nivolumab may be de-escalated within a treatment arm for further evaluation. The DRC is responsible for evaluating the safe dose, and for safeguarding the interests of study participants and monitoring the overall conduct of the study. In Part 2, treatment arms deemed safe may be expanded at the recommended part 2 dose (RP2D) with approximately 14 additional subjects to obtain a total of 20 nivolumab-treated subjects (defined as receiving ≧1 dose of nivolumab) in each treatment arm. Safety and tolerability is further assessed and anti-tumor activity is explored. Since the primary population for the pancreas arms is subjects with no prior chemotherapy, surgery or radiation therapy, enrollment in Part 2 for Panc Arm A continues until 20 such subjects have been treated with at least one dose of nivolumab.

Subjects are treated with nivolumab at the given dose level administered intravenously over 60 minutes prior to chemotherapy according to protocol-defined treatment regimen. nab-Paclitaxel, and gemcitabine should be stored, reconstituted, and administered according to the local prescribing information, at the protocol defined dose and frequency.

In both Part 1 and Part 2, subjects receive treatment according to their assigned treatment arm with nivolumab and a fixed starting dose of chemotherapy. The following combination regimens are administered:
Panc Arm A and B: sequential enrollment in Part 1
   Panc Arm A: nab-paclitaxel 125 mg/m2 on Days 1, 8 and 15, and nivolumab on Days 1 and 15 of each 28 day cycle.
   Panc Arm B: If Panc Arm A is deemed safe (Part 1, DLT assessment), nab-paclitaxel 125 mg/m2 on Days 1, 8 and 15, gemcitabine 1000 mg/m2 on Days 1, 8 and 15, and nivolumab on Days 1 and 15 of each 28-day cycle.

In Part 1, the combination regimen is assessed for DLTs. In Panc treatment arms, nivolumab is given at 3 mg/kg q2 weekly in 28 day cycles (Panc Arm A and B) as dose level 1 (DL1), and if needed, de-escalated to a 1 mg/kg q3 weekly (DL-1). If further reduction of nivolumab is needed in a treatment arm, additional de-escalation to 0.3 mg/kg q3 weekly may occur. If a safe dose of nivolumab is identified in Part 1 for a given treatment arm, that combination regimen may move forward in Part 2 (RP2D). The dose levels for Panc A and B are shown in Table 2.

TABLE 2

| | | nab- | Gemcitabine | Nivolumab D* |
|---|---|---|---|---|
| | | Paclitaxel | D* 1, | 1, 15 of |
| | | D* 1, 8, 15 | 8, 15 of | 28 day |
| Pane | Dose | of 28 day | 28 day | cycle starting |
| Arm | Level | cycle | cycle | Cycle 1 |
| A | 1 | 125 mg/m² | — | 3 mg/kg |
| | −1 | 125 mg/m² | — | 1 mg/kg |
| | −2 | 125 mg/m² | — | 0.3 mg/kg |
| B | 1 | 125 mg/m² | 1000 mg/m² | 3 mg/kg |
| | −1 | 125 mg/m² | 1000 mg/m² | 1 mg/kg |
| | −2 | 125 mg/m² | 1000 mg/m² | 0.3 mg/kg |

D* = day

In Parts 1 and 2, treatment regimens are given until RECIST 1.1 disease progression, unacceptable toxicity or withdrawal of consent. Subjects will be permitted to continue nivolumab treatment beyond initial RECIST 1.1 defined progressive disease as long as they meet the following criteria:
Continue to meet all other study protocol eligibility criteria
Investigator assessed clinical benefit and do not have rapid disease progression or clinical deterioration
Stable performance status
Tolerance to study drug
Treatment beyond progression will not delay an imminent intervention to prevent serious complications of disease progression (eg, brain metastases)

Overview of Efficacy Assessments:

All subjects are evaluated for tumor response and progression according to RECIST 1.1 guidelines (investigator assessment). Baseline tumor measurements are determined from the radiologic evaluation performed within 28 days before Cycle 1 Day 1. Thereafter, radiologic evaluation is performed every 6 weeks (±3 days) from Cycle 1 Day 1 for the first 24 weeks (first 4 assessments) and then every 8 weeks until disease progression, start of a new anticancer therapy or withdrawal of consent from the entire study, whichever comes first. Those subjects entering the follow-up period continue to have CT scans in accordance with local standard of care until withdrawal of consent, lost to follow-up, death, or termination of the study by the sponsor. In the follow-up period, anti-cancer treatment administered following the last dose of investigational product ([IP]: nab-paclitaxel, gemcitabine, or nivolumab) and survival is followed every 3 months (±7 days), by telephone or subject visit, or review of records until death or withdrawal of consent. Subjects are followed for overall survival for up to 1 year after the last subject is enrolled.

Overview of Safety Assessments:

All subjects are monitored for adverse events, starting from the time the subject signs the informed consent form. Treatment-emergent adverse events (TEAEs) are captured through 28 days after the last dose of the IPs. In addition, any AE with an onset date more than 28 days and up to 100 days after the last dose of the IPs is also considered a TEAE. Physical examination (source documented only), vital signs, laboratory assessments (eg, serum chemistry, hematology), and ECOG performance status are monitored regularly. Preventative measures are taken to avoid pregnancy in trial subjects or their partners, and females of child-bearing potential will have regular pregnancy testing. In Part 1, DLTs are assessed during the first two cycles of nivolumab. In the follow-up period, safety continues to be followed, either via phone or office visit, every 30 days (±7 days) and on Day 100 for adverse events. Laboratory assessment of complete blood counts and serum chemistry is performed at Day 60 (±7 days) and Day 100 (±7 days).

Subjects:

The study is planned to enroll approximately 36 to 138 subjects with histologically or cytologically confirmed cancer, including pancreatic cancer (adenocarcinoma of the pancreas with 1 prior systemic chemotherapy (Panc Arm A dose-limiting toxicity assessment, Part 1 only); or no prior chemotherapy, surgery or radiation therapy for locally advanced or metastatic disease (Panc Arm A, Part 2 and Panc Arm B)). In Part 1, approximately 6-12 subjects are planned to be enrolled in each treatment arm, with approximately 14 (20 for Panc Arm A) additional subjects per arm in Part 2.

Study Population—Inclusion Criteria:

Subjects must satisfy the following criteria to be enrolled in both Part 1 and Part 2 of the study:

1. Subject is male or female, ≧18 years old at the time of signing the informed consent form (ICF).

2. Subject has a confirmed diagnosis of advanced unresectable solid tumors in the target subject population within the parameters mentioned:
Subject has a definitive histologically or cytologically confirmed locally advanced or metastatic adenocarcinoma of the pancreas. Subjects with islet cell neoplasms are excluded.
Panc Arm A (Part 1, DLT assessment only): Subjects must have received 1 prior systemic chemotherapy regimen for locally advanced or metastatic disease.
Panc Arm A (Part 2 only) and Panc Arm B (Part 1 and 2): Subjects must have received no previous radiotherapy, surgery, chemotherapy or investigational therapy for the treatment of locally advanced or metastatic disease. Subjects having received cytotoxic doses of gemcitabine or any other chemotherapy in the adjuvant setting are not eligible for inclusion. Prior treatment with 5-FU or gemcitabine administered as a radiation sensitizer in the adjuvant setting is allowed, but ≧6 months must have elapsed since completion of the last dose and no lingering toxicities may be present. Initial diagnosis of metastatic disease must have occurred ≦6 weeks prior to randomization in the study.

3. Subject has Eastern Cooperative Oncology Group (ECOG) performance status 0 or 1.

4. Subject has no other malignancy within 5 years, except non-melanoma skin cancer, cervical intraepithelial neoplasia, or in-situ cervical cancer.

5. Subject has the following laboratory values at screening:
WBCs≧2000/uL,
Absolute neutrophil count (ANC)≧1.5×109/L,
Hemoglobin (Hgb)≧90 g/L,
Platelets (plt)≧100×109/L,
Potassium within normal range, or correctable with supplements,
AST and ALT≦2.5× Upper Limit of Normal (ULN) or ≦3.0×ULN if liver tumor is
present,
Serum total bilirubin≦1.5×ULN (except in subjects with Gilbert's who may have
serum bilirubin<3.0×ULN),
Serum creatinine≦1.5×ULN, or 24-hr clearance≧60 mL/min,
Normal coagulation [prothrombin time and partial thromboplastin time within normal limits (±15%)].

6. Subject has resting baseline oxygen saturation by pulse oximetry of ≧92% at rest.

7. Females of child-bearing potential (defined as a sexually mature woman who: 1) has not undergone a hysterectomy (the surgical removal of the uterus) or bilateral oophorectomy (the surgical removal of both ovaries) or, 2) has not been naturally postmenopausal for at least 24 consecutive months (ie, has had menses at any time during the preceding 24 consecutive months) must:

Agree to use, and be able to comply with, highly effective contraception (failure rate less than 1% per year) without interruption while on study treatment and for 23 weeks after discontinuation; and

Have a negative serum pregnancy test result (minimum sensitivity 25 IU/L or equivalent units of β-hCG) at screening and 24 hours prior to the start of any IP and agree to ongoing pregnancy testing during the course of the study, and after the end of study therapy.

Women must not be breastfeeding. (Females should not be breastfeeding while receiving nivolumab and up to 18 weeks from the last dose of nivolumab).

8. Male subjects agree to use a condom during sexual contact with a pregnant female or a female of childbearing potential while participating in the study, during dose interruptions and for 31 weeks following IP discontinuation, even if he has undergone a successful vasectomy.

9. Subject or his/her legally authorized representative or guardian understands and voluntarily signs an informed consent document prior to any study related assessments/procedures are conducted.

10. Subject is able to adhere to the study visit schedule and other protocol requirements.

Study Population—Exclusion Criteria:

The presence of any of the following will exclude a subject from enrollment:

1. Subject has a history of allergy or hypersensitivity to any study drugs or their excipients.

2. Subject has symptomatic brain metastases, spinal cord compression, or intractable back pain due to compression of destructive mass.

3. Subject has active, known or suspected autoimmune disease, including systemic lupus erythematodes, Hashimotos thyroiditis, scleroderma, polyarteritis nodosa or auto-immune hepatitis. Subjects with Type I diabetes mellitus, hypothyroidism only requiring hormone replacement, skin disorders (such as vitiligo, psoriasis or alopecia) not requiring systemic treatment, or conditions not expected to recur in the absence of an external trigger are permitted to enroll.

4. Subject is currently receiving or requires treatment with immunosuppressive agents or immunosuppressive doses of systemic corticosteroids (unless used to treat drug-related adverse events). Topical, ocular, intra-articular, intranasal, inhalational corticosteroids (with minimal systemic absorption), and some uses of systemic corticosteroids may be permitted.

5. Subject has any peripheral neuropathy≧NCI CTCAE Grade 2 at randomization/enrollment.

6. Subject has a history of interstitial lung disease, history of slowly progressive dyspnea and unproductive cough, sarcoidosis, silicosis, idiopathic pulmonary fibrosis, pulmonary hypersensitivity pneumonitis or multiple allergies. Any lung disease that may interfere with the detection or management of suspected drug-related pulmonary toxicity.

7. Subject has a high cardiovascular risk, including, but not limited to, recent coronary stenting or myocardial infarction in the past year.

8. Subject has unstable angina, a significant cardiac arrhythmia, or New York Heart Association Class 3 or 4 congestive heart failure.

9. Subject has a history of peripheral artery disease (eg, claudication, Leo Buerger's disease).

10. Subject has had major surgery, other than diagnostic surgery, within 4 weeks prior to treatment in study.

11. Subject has known acute or chronic pancreatitis.

12. Subject has persistent diarrhea, malabsorption, or known sub-acute bowel obstruction≧NCI CTCAE Grade 2, despite medical management.

13. Subject has active, uncontrolled bacterial, viral, or fungal infection(s) requiring systemic therapy.

14. Subject has any history of testing positive for Human Immunodeficiency Virus (HIV) or known acquired immunodeficiency disorder (AIDS).

15. Subject has historical or active infection with hepatitis B, or hepatitis C.

16. Subject is pregnant or breast-feeding.

17. Subject is currently enrolled in any other clinical protocol or investigational trial that involves administration of experimental therapy and/or therapeutic devices, or investigational drug.

18. Subject is currently using or use within 6 months of illicit drugs.

19. Subject has any significant medical condition, laboratory abnormality, or psychiatric illness that would prevent the subject from participating in the study.

20. Subject has any condition, including the presence of laboratory abnormalities, which places the subject at unacceptable risk if he/she were to participate in the study.

21. Subject has any condition that confounds the ability to interpret data from the study.

### Example 3. Nab-Paclitaxel with Nivolumab (BMS-936558) and Carboplatin in Non-Small Lung Cell Cancer (NSCLC)

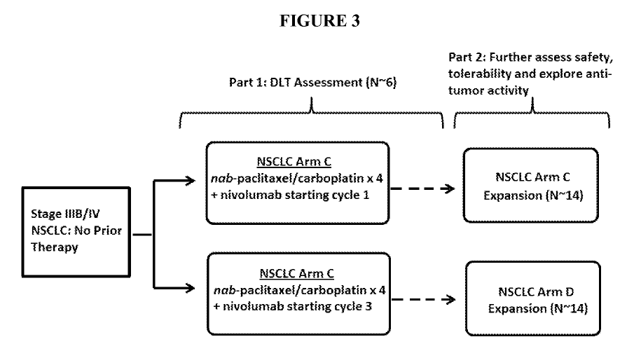

A Phase 1, open-label, multicenter, safety study of nab-paclitaxel based chemotherapy regimens administered prior to and/or in combination with nivolumab in non-small lung cell cancer (NSCLC) is performed. This study includes two treatment arms (NSCLC Arm C and D). In this study, subjects in NSCLC Arms C and D received nab-paclitaxel on days 1, 8 and 15, and carboplatin on Day 1 of each 21 day cycle from Cycles 1 to 4. Subjects in NSCLC Arm C received nivolumab on D15 starting in Cycle 1, and subjects in NSCLC Arm D started in cycle 3. Nivolumab was administered as monotherapy from Cycle 5 on. The study design is shown in FIG. 3.

Objectives:

The primary objective of the study was to evaluate the safety of nab-paclitaxel therapies in combination with carboplatin and nivolumab administered at different doses and treatment schedules in non-small cell lung cancer (NSCLC). Enrollment in each treatment arm was conducted simultaneously. The primary objective was to evaluate the dose limiting toxicities (DLTs) of each combination regimen and to characterize the safety profile of each combination regimen. The secondary objectives were to explore the anti-tumor activity and assess the safety and tolerability of the proposed regimens for each of the tumor types/indications.

Study Design:

In Part I, the two NSCLC arms (Arms C and D) were initiated at the same time. Subjects were assigned randomly between treatment arms of a tumor type/indication whenever both treatment arms are enrolling. An interactive response technology (IRT) system was used to ensure the central randomization of subjects.

DLTs are assessed during the first 2 cycles of nivolumab. Subjects were enrolled in each treatment arm to ensure up to 6 evaluable subjects in each arm at the given dose level (DL) for assessment of DLT. If ≦1 DLT occurred at a given DL in 6 subjects, and the DL was deemed safe by the Data Review Committee (DRC), this dose was the recommended Part 2 dose (RP2D).

There were no dose escalations of nivolumab in any treatment arm. If >1 DLT occurred with a DL within a treatment arm and the DL was deemed unsafe by the DRC, the dose of nivolumab may be de-escalated within a treatment arm for further evaluation. The DRC was responsible for evaluating the safe dose, and for safeguarding the interests of study participants and monitoring the overall conduct of the study.

Subjects were treated with nivolumab at the given dose level administered intravenously over 60 minutes prior to chemotherapy according to protocol-defined treatment regimen. nab-Paclitaxel, and carboplatin was stored, reconstituted, and administered according to the local prescribing information, at the protocol defined dose and frequency.

Subjects received treatment according to their assigned treatment arm with nivolumab and a fixed starting dose of chemotherapy. The following combination regimens were administered:
NSCLC Arms C and D: parallel enrollment in Parts 1 and 2
   NSCLC Arm C: nab-paclitaxel 100 mg/m2 on Days 1, 8 and 15 and carboplatin AUC 6 on Day 1 (Cycle 1 to 4 only) of each 21 day cycle;
nivolumab on Day 15 of each 21 day cycle starting in Cycle 1.
   NSCLC Arm D: nab-paclitaxel 100 mg/m2 on Days 1, 8 and 15 and carboplatin AUC 6 on Day 1 (Cycles 1 to 4 only) of each 21 day cycle; nivolumab on Day 15 of each 21 day cycle starting in Cycle 3.

In NSCLC treatment arms, nivolumab was given at 5 mg/kg q3 weekly in 21 day cycles (NSCLC Arm C and D at dose level 1 (DL1), and if needed, de-escalated to 3 mg/kg q2 weekly dose level (DL-1). If further reduction of nivolumab was needed in a treatment arm, additional de-escalation to 1 mg/kg q2 weekly dose level (DL-2) may occur. The dose levels for NSCLC Arm C and D are shown in Table 3.

TABLE 3

| | | Cycles 1-4 | | Variable start |
|---|---|---|---|---|
| | | nab-Paclitaxel | Carboplatin, | Nivolumab D* 15 |
| | | D* 1, | D* 1 | of 21 day cycle |
| NSCLC | Dose | 8, 15 of 21 | of 21 day | starting Cycle 1 |
| Arm | Level | day cycle | cycle | (C) or Cycle 3 (D) |
| | 1 | 100 mg/m² | AUC 6 | 5 mg/kg |
| C | −1 | 100 mg/m² | AUC 6 | 3 mg/kg |
| | −2 | 100 mg/m² | AUC 6 | 1 mg/kg |
| | 1 | 100 mg/m² | AUC 6 | 5 mg/kg |
| D | −1 | 100 mg/m² | AUC 6 | 3 mg/kg |
| | −2 | 100 mg/m² | AUC 6 | 1 mg/kg |

D* = day;
AUC = area under the curve (mg/ml)

Treatment regimens were given until RECIST 1.1 disease progression, unacceptable toxicity or withdrawal of consent. Subjects were permitted to continue nivolumab treatment beyond initial RECIST 1.1 defined progressive disease as long as they met the following criteria:
Continue to meet all other study protocol eligibility criteria
Investigator assessed clinical benefit and do not have rapid disease progression or clinical deterioration
Stable performance status
Tolerance to study drug
Treatment beyond progression will not delay an imminent intervention to prevent serious complications of disease progression (eg, brain metastases)

Overview of Efficacy Assessments:

All subjects were evaluated for tumor response and progression according to RECIST 1.1 guidelines (investigator assessment). Baseline tumor measurements were determined from the radiologic evaluation performed within 28 days before Cycle 1 Day 1. Thereafter, radiologic evaluation was performed every 6 weeks (±3 days) from Cycle 1 Day 1 for the first 24 weeks (first 4 assessments) and then every 8 weeks until disease progression, start of a new anticancer therapy or withdrawal of consent from the entire study, whichever comes first. Those subjects entering the follow-up period continued to have CT scans in accordance with local standard of care until withdrawal of consent, lost to follow-up, death, or termination of the study by the sponsor. In the follow-up period, anti-cancer treatment was administered following the last dose of investigational product ([IP]: nab-paclitaxel, carboplatin, or nivolumab) and survival was followed every 3 months (±7 days), by telephone or subject visit, or review of records until death or withdrawal of consent. Subjects were followed for overall survival for up to 1 year after the last subject is enrolled.

Overview of Safety Assessments:

All subjects were monitored for adverse events, starting from the time the subject signs the informed consent form. Treatment-emergent adverse events (TEAEs) were captured through 28 days after the last dose of the IPs. In addition, any AE with an onset date more than 28 days and up to 100 days after the last dose of the IPs was also considered a TEAE. Physical examination (source documented only), vital signs, laboratory assessments (eg, serum chemistry, hematology), and ECOG performance status were monitored regularly. Preventative measures were taken to avoid pregnancy in trial subjects or their partners, and females of child-bearing potential had regular pregnancy testing. DLTs were assessed during the first two cycles of nivolumab. In the follow-up period, safety continued to be followed, either via phone or office visit, every 30 days (±7 days) and on Day 100 for adverse events. Laboratory assessment of complete blood counts and serum chemistry was performed at Day 60 (±7 days) and Day 100 (±7 days).

Subjects:

The study planned to enroll approximately 36 to 138 subjects with histologically or cytologically confirmed cancer, including NSCLC (Stage IIIB or IV NSCLC with no prior chemotherapy for metastatic disease and in subjects who are not candidates for curative surgery or radiation.

Study Population—Inclusion Criteria:

Subjects satisfying the following criteria were enrolled in both Part 1 and Part 2 of the study:

1. Subject is male or female, ≧18 years old at the time of signing the informed consent form (ICF).

2. Subject has a confirmed diagnosis of advanced unresectable solid tumors in the target subject population within the parameters mentioned:

NSCLC Arms C and D (Non-small Cell Lung Cancer):
Subject has definitive histologically or cytologically confirmed Stage IIIB or IV NSCLC.
Subjects must have received no previous chemotherapy or investigational therapy for the treatment of metastatic disease. Adjuvant chemotherapy is permitted providing cytotoxic chemotherapy was completed >12 months prior to randomization, without disease recurrence or progression during those 12 months.

3. Subject has Eastern Cooperative Oncology Group (ECOG) performance status 0 or 1.

4. Subject has no other malignancy within 5 years, except non-melanoma skin cancer, cervical intraepithelial neoplasia, or in-situ cervical cancer.

5. Subject has the following laboratory values at screening:
WBCs≧2000/uL,
Absolute neutrophil count (ANC)≧1.5×109/L,
Hemoglobin (Hgb)≧90 g/L,
Platelets (plt)≧100×109/L,
Potassium within normal range, or correctable with supplements,
AST and ALT≦2.5× Upper Limit of Normal (ULN) or ≦3.0×ULN if liver tumor is
present,
Serum total bilirubin≦1.5×ULN (except in subjects with Gilbert's who may have
serum bilirubin<3.0×ULN),
Serum creatinine≦1.5×ULN, or 24-hr clearance≧60 mL/min,
Normal coagulation [prothrombin time and partial thromboplastin time within normal limits (±15%)].

6. Subject has resting baseline oxygen saturation by pulse oximetry of ≧92% at rest.

7. Females of child-bearing potential (defined as a sexually mature woman who: 1) has not undergone a hysterectomy (the surgical removal of the uterus) or bilateral oophorectomy (the surgical removal of both ovaries) or, 2) has not been naturally postmenopausal for at least 24 consecutive months (ie, has had menses at any time during the preceding 24 consecutive months) must:
Agree to use, and be able to comply with, highly effective contraception (failure rate less than 1% per year) without interruption while on study treatment and for 23 weeks after discontinuation; and
Have a negative serum pregnancy test result (minimum sensitivity 25 IU/L or equivalent units of β-hCG) at screening and 24 hours prior to the start of any IP and agree to ongoing pregnancy testing during the course of the study, and after the end of study therapy.
Women must not be breastfeeding. (Females should not be breastfeeding while receiving nivolumab and up to 18 weeks from the last dose of nivolumab).

8. Male subjects agree to use a condom during sexual contact with a pregnant female or a female of childbearing potential while participating in the study, during dose interruptions and for 31 weeks following IP discontinuation, even if he has undergone a successful vasectomy.

9. Subject or his/her legally authorized representative or guardian understands and voluntarily signs an informed consent document prior to any study related assessments/procedures are conducted.

10. Subject is able to adhere to the study visit schedule and other protocol requirements.

Study Population—Exclusion Criteria:

The presence of any of the following excluded a subject from enrollment:

1. Subject has a history of allergy or hypersensitivity to any study drugs or their excipients.

2. Subject has symptomatic brain metastases, spinal cord compression, or intractable back pain due to compression of destructive mass.

3. Subject has active, known or suspected autoimmune disease, including systemic lupus erythematodes, Hashimotos thyroiditis, scleroderma, polyarteritis nodosa or auto-immune hepatitis. Subjects with Type I diabetes mellitus, hypothyroidism only requiring hormone replacement, skin disorders (such as vitiligo, psoriasis or alopecia) not requiring systemic treatment, or conditions not expected to recur in the absence of an external trigger are permitted to enroll.

4. Subject is currently receiving or requires treatment with immunosuppressive agents or immunosuppressive doses of systemic corticosteroids (unless used to treat drug-related adverse events). Topical, ocular, intra-articular, intranasal, inhalational corticosteroids (with minimal systemic absorption), and some uses of systemic corticosteroids may be permitted.

5. Subject has any peripheral neuropathy≧NCI CTCAE Grade 2 at randomization/enrollment.

6. Subject has a history of interstitial lung disease, history of slowly progressive dyspnea and unproductive cough, sarcoidosis, silicosis, idiopathic pulmonary fibrosis, pulmonary hypersensitivity pneumonitis or multiple allergies. Any lung disease that may interfere with the detection or management of suspected drug-related pulmonary toxicity.

7. Subject has a high cardiovascular risk, including, but not limited to, recent coronary stenting or myocardial infarction in the past year.

8. Subject has unstable angina, a significant cardiac arrhythmia, or New York Heart Association Class 3 or 4 congestive heart failure.

9. Subject has a history of peripheral artery disease (eg, claudication, Leo Buerger's disease).

10. Subject has had major surgery, other than diagnostic surgery, within 4 weeks prior to treatment in study.

11. Subject has known acute or chronic pancreatitis.

12. Subject has persistent diarrhea, malabsorption, or known sub-acute bowel obstruction≧NCI CTCAE Grade 2, despite medical management.

13. Subject has active, uncontrolled bacterial, viral, or fungal infection(s) requiring systemic therapy.

14. Subject has any history of testing positive for Human Immunodeficiency Virus (HIV) or known acquired immunodeficiency disorder (AIDS).

15. Subject has historical or active infection with hepatitis B, or hepatitis C.

16. Subject is pregnant or breast-feeding.

17. Subject is currently enrolled in any other clinical protocol or investigational trial that involves administration of experimental therapy and/or therapeutic devices, or investigational drug.

18. Subject is currently using or use within 6 months of illicit drugs.

19. Subject has any significant medical condition, laboratory abnormality, or psychiatric illness that would prevent the subject from participating in the study.

20. Subject has any condition, including the presence of laboratory abnormalities, which places the subject at unacceptable risk if he/she were to participate in the study.

21. Subject has any condition that confounds the ability to interpret data from the study.

Results:

As part of the ongoing study, twelve subjects have been enrolled in Part I of NSCLC Arm C. Nine subjects received nivolumab and three subjects did not receive nivolumab. Each of the nine subjects receiving nivolumab exhibited either partial response (6) or stable disease (3). Of the three subjects who did not receive nivolumab, stable disease was observed in one subject, and two subjects were non-evaluable (NE) due to no data or incomplete data. No death nor dose limiting toxicity were reported for the twelve subjects who have been enrolled in Part I of NSCLC Arm C. One subject who did not receive nivolumab experienced three severe adverse events (acute respiratory failure, chronic obstructive pulmonary disease, and cardiac failure congestive). As such, the severe adverse events were determined by the investigator to be unrelated to administration of nivolumab or combinations comprising nivolumab.

### Example 4. Nab Paclitaxel with MPDL3280A in Metastatic Breast Cancer

Objectives:

The primary objective of the study is to evaluate the safety and tolerability of nab-paclitaxel in combination with MPDL3280A (atezolizumab). The secondary objectives are to evaluate pharmacokinetics and clinical activity of the combination for the treatment of metastatic breast cancer.

Study Design:

The study design is as described in Example 1, above, except MPDL3280A is administered in place of nivolumab. Additionally, certain subjects can: 1) receive 125 mg/m² of nab-paclitaxel q1w (days 1, 8 and 15) with 800 mg of MPDL3280A q2w (days 1 and 15) for 3 weeks in 4-week cycles, which is continued until loss of clinical benefit; and 2) receive 100 mg²/m² of nab-paclitaxel on days 1, 8 and 15, with 840 mg MPDL3280A on days 1 and 15, on a 28-day cycle.

### Example 5. Nab-Paclitaxel in Combination with MEDI4736 in Stage I-III Triple Negative Breast Cancer

A safety and efficacy study of nab-paclitaxel in combination with MEDI4736 (durvalumab) in subjects with stage I-III triple-negative breast cancer is performed.

In this study, subjects receive MEDI4736 intravenously. Routine, standard of care chemotherapy is given together with MEDI4736 and includes weekly nab-paclitaxel×12 treatments followed by every two-week doxorubicin, cyclophosphamide (ddAC)×4 treatments.

### Example 6. Nab-Paclitaxel in Combination with MEDI4736 in Metastatic Breast Cancer

A study of nab-paclitaxel with MEDI4736 (durvalumab) in subjects with metastatic breast cancer (HER-2-negative recurrent metastatic breast cancer after one prior metastatic breast cancer treatment regimen) is performed.

Subjects receive nab-paclitaxel 125 mg/m² weekly for every 2 out of 3 weeks and 1500 mg of MEDI4736 every 2 weeks.

### Example 7. Nab Paclitaxel in Combination with Carboplatin and MPDL3280A in Metastatic Breast Cancer

A study of nab-paclitaxel with carboplatin and MPDL3280A in subjects with metastatic breast cancer (HER-2-negative recurrent metastatic breast cancer or triple negative metastatic breast cancer) is performed.

Subjects receive: 1) nab-paclitaxel 125 mg/m² weekly for every 2 out of 3 weeks in combination with 1.5 AUC of carboplatin weekly for every 2 out of 3 weeks and 1200 mg of MPDL3280A on day 1 of a 21 day cycle; or 2) nab-paclitaxel 100 mg/m² on days 1, 8 and 15 of a 21-day cycle, in combination with 6 AUC of carboplatin on day 1 of a 21-day cycle, for 4-6 cycles, and 1200 mg IV of MPDL3280A on day 1 of a 21-day cycle.

### Example 8. Nab-Paclitaxel with MPDL3280A and Carboplatin in Non-Small Cell Lung Cancer (NSCLC)

Study Design:

The study design is as described in Example 3, above, except MPDL3280A is administered in place of nivolumab. Additionally, certain subjects can be administered: 1) 100 mg/m² nab-paclitaxel on days 1, 8, and 15 of a 21 day cycle, carboplatin AUC 6 on day 1 of a 21 day cycle (for 4-6 cycles) and 1200 mg MPDL3280A on day 1 of a 21 day cycle; 2) 200 mg/m² nab-paclitaxel on days 1 and 8 of a 21 day cycle, carboplatin AUC 6 on day 1 of a 21 day cycle (for 4-6 cycles) and 1200 mg MPDL3280A on day 1 of a 21 day cycle; or 3) 100 mg/m² nab-paclitaxel intravenously weekly (IV q1w), carboplatin AUC 6 intravenously every three weeks (IV q3w) and 800 mg every two weeks (q2w), 1200 mg every three weeks (q 3w) or 15 mg/kg q 3w MPDL3280A.

### Example 9. Nab-Paclitaxel in Combination with Carboplatin and Pembrolizumab in Non-Small Cell Lung Cancer

A study of nab-paclitaxel with carboplatin and pembrolizumab in subjects with advanced non-small cell lung cancer is performed.

Subjects receive: nab-paclitaxel 100 mg/m² on days 1, 8 and 15 of a 21-day cycle in combination with 6 AUC of carboplatin on day 1 of a 21-day cycle, and 2 mg/kg of pembrolizumab on day 1 of a 21 day cycle, for 4 cycles.

### Example 10. Nab Paclitaxel in Combination with Gemcitabine and Pembrolizumab in Non-Small Cell Lung Cancer

A study of nab-paclitaxel with gemcitabine and pembrolizumab in subjects with advanced non-small cell lung cancer is performed.

Subjects receive: nab-paclitaxel 125 mg/m² on days 1 and 8 of a 21-day cycle in combination with 1000 mg gemcitabine on days 1 and 8 of a 21-day cycle, and 2 mg/kg of pembrolizumab (IV) on day 1 of a 21 day cycle.

### Example 11. Nab-Paclitaxel with MEDI4736 (Durvalumab) in Pancreatic Cancer

A study of nab-paclitaxel with MEDI4736 (druvalumab) in subjects with pancreatic cancer is performed.

In particular, 1500 mg of MEDI4736 (durvalumab) is administered at day 1 of a 21 day cycle, or at day 1 and day 15 of a 21 day cycle, and nab-paclitaxel is administered at 125 mg/m² week for 2 out of 3 weeks of a 21 day cycle. In addition, in one arm, following one, two, three or four cycles, patients continue with MEDI4736 at the same dose and receive radiation treatment.

### Example 12. Nab-Paclitaxel in Combination with Gemcitabine, MEDI4736 (Druvalumab) and Radiation in Pancreas Ductal Adenocarcinoma (PDAC)

A study of nab-paclitaxel with MEDI4736 (durvalumab) and radiation in subjects with pancreas ductal adenocarcinoma is performed.

In particular, 2-20 mg/kg of MEDI4736 (durvalumab), 125 mg/m² nab-paclitaxel and 1000 mg/m² is administered. MEDI4736 is adminstered every two weeks or every four weeks. Gemcitabine and nab-paclitaxel are administered weekly. Each cycle lasts 3 or 4 weeks. Following two cycles, subjects can continue with MEDI4736 at the same dose and receive radiation treatment.

### Example 13. Nab Paclitaxel in Combination with Pembrolizumab and Gemcitabine in Metastatic Pancreatic Adenocarcinoma

A study of nab-paclitaxel in combination with pembrolizumab and gemcitabine in subjects with metastatic pancreatic adenocarcinoma is performed.

In this study, subjects receive pembrolizumab 2 mg/kg administered intravenously over 30 minutes every 3 weeks, gemcitabine 1000 mg/m² and nab-paclitaxel 125 mg/m² on day 1 and day 8 every 21 days.

What is claimed is:

1. A method of treating a proliferative disease in an individual comprising administering to the individual:
a) an effective amount of a composition comprising nab-paclitaxel, and
b) an effective amount of an anti-PD-1 antibody, wherein the proliferative disease is lung cancer, pancreatic cancer or breast cancer.

2. The method of claim 1, wherein the composition in a) comprises ABRAXANE®.

3. The method of claim 1 or 2, wherein the anti-PD-1 antibody is nivolumab.

4. The method according to any one of claims 1-3, further comprising administering to the individual an effective amount of a platinum-based agent.

5. The method of claim 4, wherein the platinum-based agent is carboplatin.

6. The method of claim 4, wherein the platinum-based agent is cisplatin.

7. The method according to any one of claim 1-6, wherein the composition in a) and the anti-PD-1 antibody are administered concurrently.

8. The method according to any one of claim 1-6, wherein the composition in a) and the anti-PD-1 antibody are administered sequentially.

9. The method according to any one of claim 1-6, wherein the composition in a) and the anti-PD-1 antibody are administered simultaneously.

10. The method according to any one of claim 4-6, wherein the composition in a), the anti-PD-1 antibody and the platinum-based agent are administered concurrently.

11. The method according to any one of claim 4-6, wherein the composition in a), the anti-PD-1 antibody and the platinum-based agent are administered concurrently are administered sequentially.

12. The method according to any one of claim 4-6, wherein the composition in a), the anti-PD-1 antibody and the platinum-based agent are administered concurrently are administered simultaneously.

13. A method of treating a proliferative disease in an individual comprising administering to the individual:
a) an effective amount of a composition comprising nanoparticles comprising a taxane and a carrier protein, and
b) an effective amount of at least one other agent that antagonizes a PD-1 pathway in a cell.

14. The method according to claim 13, wherein said other agent antagonizes PD-1 or a ligand of PD-1.

15. The method according to claim 13, wherein said other agent is a PD-1 antagonist.

16. The method according to claim 13-15, wherein said other agent is an antibody.

17. The method according to claim 16, wherein said antibody is a monoclonal antibody.

18. The method according to claim 16, wherein said antibody is a humanized antibody or fully human antibody.

19. The method according to claim 16, wherein said antibody comprises an immunoglobulin G (IgG) antibody.

20. The method according to claim 19, wherein said IgG antibody is an IgG4 antibody.

21. The method according to claim 15, wherein said PD-1 antagonist is selected from the group consisting of AMP-224, BMS-936559, MEDI4736, MSB0010718C, MPDL3280A, nivolumab, AMP-514, pembrolizumab (MK-3475), REGN2810, PDR001, BGB-A317, and pidilizumab (CT-011).

22. The method according to claim 13, wherein said other agent is an antagonist of PD-1.

23. The method according to claim 22, wherein said antagonist of PD-1 is an anti-PD-1 antibody.

24. The method according to claim 23, wherein said anti-PD-1 antibody is nivolumab, AMP-514, pembrolizumab (MK-3475), REGN2810, PDR001, BGB-A317, or pidilizumab (CT-011).

25. The method of claim 13, wherein said other agent is an antagonist of PD-L1.

26. The method of claim 25, wherein said antagonist of PD-L1 is an anti-PD-L1 antibody.

27. The method of claim 26, wherein said anti-PD-L1 antibody is BMS-936559, MSB0010718C, MPDL3280A, and MEDI4736.

28. The method according to claim 15, wherein said PD-1 pathway antagonist comprises a fusion protein.

29. The method of claim 28, wherein said fusion protein comprises at least a portion of an antibody.

30. The method according to claim 29, wherein said antibody is an immunoglobulin G antibody.

31. The method according to claim 29, wherein said fusion protein comprises an Fc region of said antibody.

32. The method according to claim 28, wherein said fusion protein comprises at least a portion of a PD-1 ligand.

33. The method according to claim 32, wherein said PD-1 ligand is PD ligand 2 (PD-L2).

34. The method according to claim 28, wherein said fusion protein comprises an extracellular domain of said PD-1 ligand.

35. The method according to claim 28, wherein said fusion protein is AMP-224.

36. The method according to claim 13, further comprising administering to said individual a chemotherapeutic agent.

37. The method according to claim 36, wherein said chemotherapeutic agent is a platinum-based agent.

38. The method according to claim 37, wherein said platinum-based agent is carboplatin.

39. The method according to claim 37, wherein said platinum-based agent is cisplatin.

40. The method according to claim 36, wherein said chemotherapeutic agent is a nucleoside analog.

41. The method according to claim 40, wherein said nucleoside analog is gemcitabine.

42. The method according to any one of claims 13-41, wherein said proliferative disease is cancer.

43. The method according to claim 42, wherein said cancer is breast cancer.

44. The method according to claim 43, wherein said individual is negative for ER, PR, or HER2.

45. The method according to claim 43, wherein said individual is negative for ER, PR, and HER2.

46. The method according to claim 43, wherein said breast cancer is a metastatic breast cancer.

47. The method according to claim 43, wherein said breast cancer is a recurrent breast cancer.

48. The method according to claim 42, wherein said cancer is a pancreatic cancer.

49. The method according to claim 42, wherein said cancer is a lung cancer.

50. The method according to claim 49, wherein said lung cancer is a non-small cell lung cancer (NSCLC).

51. The method according to claim 50, wherein said NSCLC is a stage III or stage IV NSCLC.

52. The method according to claim 50, wherein said NSCLC is a stage IIIB NSCLC.

53. The method according to claim 13, wherein said composition comprising nanoparticles comprising taxane and said other agent are administered simultaneously.

54. The method according to claim 13, wherein said composition comprising nanoparticles comprising taxane and said other agent are administered sequentially.

55. The method according to claim 13, wherein said composition comprising nanoparticles comprising taxane and said other agent are administered concurrently.

56. The method according to claim 36, wherein said composition comprising nanoparticles comprising taxane, said other agent, and said chemotherapeutic agent are administered simultaneously.

57. The method according to claim 26, wherein said composition comprising nanoparticles comprising taxane, said other agent, and said chemotherapeutic agent are administered sequentially.

58. The method according to claim 36, wherein said composition comprising nanoparticles comprising taxane, said other agent, and said chemotherapeutic agent are administered concurrently.

59. The method according to any one of claims 12-58, wherein said taxane is paclitaxel.

60. The method according to any one of claims 13-59, wherein said nanoparticles in said composition have an average diameter of less than or equal to about 200 nm.

61. The method according to any one of claims 13-60, wherein said carrier protein is albumin.

62. The method according to claim 61, wherein said albumin and said taxane in said nanoparticle composition has a weight ratio between about 1:1 to about 18:1.

63. The method according to claim 61, wherein said albumin and said taxane in said nanoparticle composition has a weight ratio between about 1:1 to about 9:1.

64. The method of claim 61, wherein said albumin and said taxane in said nanoparticle composition has a weight ratio of about 9:1.

65. The method according to any one of claims 13-64, wherein said composition comprising nanoparticles comprising a taxane and a carrier protein comprises nab-paclitaxel.

66. The method according to any one of claims 13-65, wherein said composition comprising nanoparticles comprising a taxane and a carrier protein comprises ABRAXANE®.

67. The method according to any one of claims 13-66, wherein said at least one other agent that antagonizes a PD-1 pathway in a cell is nivolumab.

68. The method according to any one of claims 13-67, wherein said individual is a human.

69. A kit comprising: a) a composition comprising nanoparticles comprising a taxane and a carrier protein, and b) an effective amount of at least one other agent that antagonizes a PD-1 pathway in a cell.

70. A pharmaceutical composition comprising: a) a composition comprising nanoparticles comprising a taxane and a carrier protein, and b) an effective amount of at least one other agent that antagonizes a PD-1 pathway in a cell.

71. A method of treating breast cancer in an individual comprising administering to the individual:
a) an effective amount of a composition comprising nanoparticles comprising a taxane and a carrier protein, and
b) an effective amount of at least one other agent that antagonizes a PD-1 pathway in a cell.

72. The method according to claim 71, wherein said other agent is a PD-1 antagonist.

73. The method according to claim 71, wherein said other agent is an anti-PD-1 antibody.

74. The method according to claim 73, wherein said anti-PD-1 antibody is selected from the group consisting of nivolumab, AMP-514, pembrolizumab (MK-3475), REGN2810, PDR001, BGB-A317, and pidilizumab (CT-011).

75. The method according to any one of claims 71-74, wherein said taxane is paclitaxel.

76. The method according to any one of claims 71-74, wherein said carrier protein is albumin.

77. The method according to any one of claims 71-74, wherein said composition comprising nanoparticles comprising a taxane and a carrier protein comprises nab-paclitaxel.

78. The method according to any one of claims 71-77, wherein said composition comprising nanoparticles comprising a taxane and a carrier protein comprises ABRAXANE®.

79. The method according to any one of claims 71-78, wherein said other agent is nivolumab.

80. The method according to any one of claims 71-79, wherein said breast cancer is a Her2(−) breast cancer.

81. The method according to any one of claims 71-80, wherein said breast cancer is a recurrent breast cancer.

82. The method according to any one of claims 71-81, wherein said breast cancer is a metastatic breast cancer.

83. A method of treating pancreatic cancer in an individual comprising administering to the individual:
a) an effective amount of a composition comprising nanoparticles comprising a taxane and a carrier protein, and
b) an effective amount of at least one other agent that antagonizes a PD-1 pathway in a cell.

84. The method according to claim 83, wherein said other agent is a PD-1 antagonist.

85. The method according to any one of claims 83-84, wherein said other agent is an anti-PD-1 antibody.

86. The method according to claim 85, wherein said anti-PD-1 antibody is selected from the group consisting of nivolumab, AMP-514, pembrolizumab (MK-3475), REGN2810, PDR001, BGB-A317, and pidilizumab (CT-011).

87. The method according to any one of claims 83-86, wherein said taxane is paclitaxel.

88. The method according to any one of claims 83-87, wherein said carrier protein is albumin.

89. The method according to any one of claims 83-88, wherein the composition comprising nanoparticles comprising a taxane and a carrier protein comprises nab-paclitaxel.

90. The method according to any one of claims 83-89, wherein the composition comprising nanoparticles comprising a taxane and a carrier protein comprises ABRAXANE®.

91. The method according to any one of claims 83-90, wherein said other agent is nivolumab.

92. The method according to any one of claims 83-91, further comprising administering a chemotherapeutic agent.

93. The method according to claim 92, wherein said chemotherapeutic agent is a nucleoside analog.

94. The method according to claim 93, wherein said nucleoside analog is gemcitabine.

95. A method of treating lung cancer in an individual comprising administering to the individual:
a) an effective amount of a composition comprising nanoparticles comprising a taxane and a carrier protein, and
b) an effective amount of at least one other agent that antagonizes a PD-1 pathway in a cell.

96. The method according to claim 95, wherein said other agent is a PD-1 antagonist.

97. The method according to any one of claims 95-96, wherein said other agent is an anti-PD-1 antibody.

98. The method according to claim 97, wherein said anti-PD-1 antibody is selected from the group consisting of nivolumab, AMP-514, pembrolizumab (MK-3475), REGN2810, PDR001, BGB-A317, and pidilizumab (CT-011).

99. The method according to any one of claims 95-98, wherein said taxane is paclitaxel.

100. The method according to any one of claims 95-99, wherein said carrier protein is albumin.

101. The method according to any one of claims 95-100, wherein the composition comprising nanoparticles comprising a taxane and a carrier protein comprises nab-paclitaxel.

102. The method according to any one of claims 95-101, wherein the composition comprising nanoparticles comprising a taxane and a carrier protein comprises ABRAXANE®.

103. The method according to any one of claims 95-102, wherein said other agent is nivolumab.

104. The method according to any one of claims 95-103, further comprising administering a chemotherapeutic agent.

105. The method according to claim 104, wherein said chemotherapeutic agent is a platinum-based agent.

106. The method according to claim 105, wherein said platinum-based agent is carboplatin.

107. The method according to claim 105, wherein said platinum-based agent is cisplatin.

108. The method according to any one of claims 95-107, wherein said lung cancer is a non-small cell lung cancer (NSCLC).

109. The method according to any one of claims 71-108, wherein said other agent is an antagonist of a PD-1 ligand.

110. The method according to claim 109, wherein said antagonist of a PD-1 ligand is an antagonist of PD-L1.

111. The method according to claim 110, wherein said antagonist of PD-L1 is an anti-PD-L1 antibody.

112. The method according to claim 111, wherein said antagonist of PD-L1 is selected from the group consisting of BMS-936559, MEDI4736, MSB0010718C and MPDL3280A.

113. The method according to claim 109, wherein said antagonist of a PD-1 ligand is selected from the group consisting of AMP-224, BMS-936559, MEDI4736, MSB0010718C and MPDL3280A.

114. The method according to claim 109, wherein said antagonist of a PD-1 ligand is an antagonist of PD-L2.

115. The method according to claim 111, wherein said antagonist of PD-L2 is an anti-PD-L2 antibody.

116. The kit according to claim 69, wherein said other agent is defined as in any one of claims 14-35.

117. The pharmaceutical composition according to claim 61, wherein said other agent is defined as in any one of claims 14-35.

118. The kit according to claim 69, wherein said nanoparticle composition is defined as in any one of claims 59-66.

119. The pharmaceutical composition according to claim 70, wherein said nanoparticle composition is defined as in any one of claims 59-66.
